(19) United States
(12) Patent Application Publication
(10) Pub. No.: US 2008/0214527 A1
(43) Pub. Date: Sep. 4, 2008

(54) Hiv Integrase Inhibitors

(76) Inventors: Takashi Kawasuji, Osaka (JP); Brian A. Johns, Durham, NC (US)

Correspondence Address: GLAXOSMITHKLINE;CORPORATE INTELLECTUAL PROPERTY, MAI B482, FIVE MOORE DR., PO BOX 13398, RESEARCH TRIANGLE PARK, NC, 27709-3398, US

(21) Appl. No.: 11/997,786

(22) PCT Filed: Jul. 28, 2006
(86) PCT No.: PCT/US06/29645

§ 371 Date: Feb. 4, 2008

Related U.S. Application Data
(60) Provisional application No. 60/705,346, filed on Aug. 4, 2005.

(51) Int. Cl. A61K 31/541; C07D 471/04; A61P 31/18; A61K 31/4375

(52) U.S. Cl. 514/222.2; 546/123; 514/300; 544/3

(57) ABSTRACT

The present invention features compounds that are HIV integrase inhibitors useful in the inhibition of HIV replication, the prevention and/or treatment of infection by HIV, and in the treatment of AIDS and/or ARC.

### BACKGROUND OF THE INVENTION

The human immunodeficiency virus (“HIV”) is the causative agent for acquired immunodeficiency syndrome (“AIDS”), a disease characterized by the destruction of the immune system, particularly of CD4⁺ T-cells, with attendant susceptibility to opportunistic infections, and its precursor AIDS-related complex (“ARC”), a syndrome characterized by symptoms such as persistent generalized lymphadenopathy, fever and weight loss. HIV is a retrovirus; the conversion of its RNA to DNA is accomplished through the action of the enzyme reverse transcriptase. Compounds that inhibit the function of reverse transcriptase inhibit replication of HIV in infected cells. Such compounds are useful in the prevention or treatment of HIV infection in humans.

A required step in HIV replication in human T-cells is the insertion by virally-encoded integrase of proviral DNA into the host cell genome. Integration is believed to be mediated by integrase in a process involving assembly of a stable nucleoprotein complex with viral DNA sequences, cleavage of two nucleotides from the 3′ termini of the linear proviral DNA and covalent joining of the recessed 3′ OH termini of the proviral DNA at a staggered cut made at the host target site. The repair synthesis of the resultant gap may be accomplished by cellular enzymes.

There is continued need to find new therapeutic agents to treat human diseases in part because of the emergence of resistance. HIV integrase is an attractive target for the discovery of new therapeutics due to its important role in viral infections, particularly HIV infections. The compounds of the present invention are inhibitors of HIV integrase and may be useful in the treatment of HIV infection.

### SUMMARY OF THE INVENTION

The present invention features compounds that are HIV integrase inhibitors and therefore are useful in the inhibition of HIV replication, the prevention and/or treatment of infection by HIV, and in the treatment of AIDS and/or ARC. The present invention features compounds of formula (I):

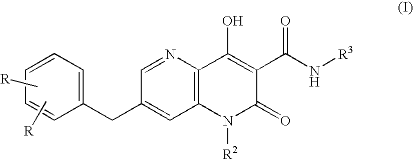

wherein:
R is halogen;
R¹ is
(a) C(O)N(R^{a}R^{b});
(b) C₃₋₆ alkynyl or C₁₋₈ alkylcarbonyl, each of which may be optionally substituted with one or more substituents independently selected from the group consisting of C₁₋₈ alkyl, C₁₋₈ haloalkyl, C₃₋₇ cycloalkyl, C₂₋₆ alkenyl, C₃₋₇ cycloalkenyl, C₃₋₆ alkynyl, halogen, CN, NO₂, OR^{a}, N(R^{a}R^{b}), S(O)ₘR^{a}, SR^{a}, OS(O)ₘR^{a}, S(O)ₘOR^{a}, OS(O)ₘOR^{a}, N(R^{a})S(O)ₘR^{b}, S(O)ₘN(R^{a}R^{b}), N(R^{a})S(O)ₘN(R^{a}R^{b}), OS(O)ₘN(R^{a}R^{b}), N(R^{a})S(O)ₘOR^{b}, C(O)R^{a}, OC(O)R^{a}, C(O)OR^{a}, OC(O)OR^{a}, N(R^{a})C(O)R^{b}, C(O)N(R^{a}R^{b}), N(R^{a})C(O)N(R^{a}R^{b}), OC(O)N(R^{a}R^{b}), N(R^{a})C(O)OR^{b}, C(NR^{a}R^{b})═N(R^{a}), N(R^{a})C(NR^{a}R^{b})═N(R^{a}), C(SR^{a})═N(R^{b}), C(OR^{a})═N(R^{b}), N(R^{a})C(SR^{a})═N(R^{b}) and heterocycle optionally substituted with oxo or R^{a}; or
(c) C₁₋₈ alkyl or C₁₋₈ alkoxy, each of which is substituted with one or more substituents independently selected from the group consisting of C₆₋₁₄ aryl, C₁₋₈ alkyl, C₁₋₈ haloalkyl, C₃₋₇ cycloalkyl, C₂₋₆ alkenyl, C₃₋₇ cycloalkenyl, C₃₋₆ alkynyl, halogen, CN, NO₂, OR^{a}, N(R^{a}R^{b}), S(O)ₘR^{a}, SR^{a}, OS(O)ₘR^{a}, S(O)ₘOR^{a}, OS(O)ₘOR^{a}, N(R^{a})S(O)ₘR^{b}, S(O)ₘN(R^{a}R^{b}), N(R^{a})S(O)ₘN(R^{a}R^{b}), OS(O)ₘN(R^{a}R^{b}), N(R^{a})S(O)ₘOR^{b}, C(O)R^{a}, OC(O)R^{a}, C(O)OR^{a}, OC(O)OR^{a}, N(R^{a})C(O)R^{b}, C(O)N(R^{a}R^{b}), N(R^{a})C(O)N(R^{a}R^{b}), OC(O)N(R^{a}R^{b}), N(R^{a})C(O)OR^{b}, C(NR^{a}R^{b})═N(R^{a}), N(R^{a})C(NR^{a}R^{b})═N(R^{a}), C(SR^{a})═N(R^{b}), C(OR^{a})═N(R^{b}), N(R^{a})C(SR^{a})═N(R^{b}) and heterocycle optionally substituted with oxo or R^{a};
R² is hydrogen, C₁₋₈ alkyl, C₁₋₈ haloalkyl, C₃₋₇ cycloalkyl, C₆₋₁₄ aralkyl, C₂₋₆ alkenyl, C₃₋₇ cycloalkenyl, C₃₋₆ alkynyl, C₆₋₁₄ aryl, or heterocycle, each of which may be optionally substituted with one or more substituents independently selected from the group consisting of C₁₋₈ alkyl, C₁₋₈ haloalkyl, C₃₋₇ cycloalkyl, C₂₋₆ alkenyl, C₃₋₇ cycloalkenyl, C₃₋₆ alkynyl, halogen, CN, NO₂, OR^{a}, N(R^{a}R^{b}), S(O)ₘR^{a}, SR^{a}, OS(O)ₘR^{a}, S(O)ₘOR^{a}, OS(O)ₘOR^{a}, N(R^{a})S(O)ₘR^{b}, S(O)ₘN(R^{a}R^{b}), N(R^{a})S(O)ₘN(R^{a}R^{b}), OS(O)ₘN(R^{a}R^{b}), N(R^{a})S(O)ₘOR^{b}, C(O)R^{a}, OC(O)R^{a}, C(O)OR^{a}, OC(O)OR^{a}, N(R^{a})C(O)R^{b}, C(O)N(R^{a}R^{b}), N(R^{a})C(O)N(R^{a}R^{b}), OC(O)N(R^{a}R^{b}), N(R^{a})C(O)OR^{b}, C(NR^{a}R^{b})═N(R^{a}), N(R^{a})C(NR^{a}R^{b})═N(R^{a}), C(SR^{a})═N(R^{b}), C(OR^{a})═N(R^{b}), N(R^{a})C(SR^{a})═N(R^{b}) and heterocycle optionally substituted with oxo or R^{a};
or optionally when R² is C₅₋₇ cycloalkyl, C₆₋₁₄ aralkyl, C₅₋₇ cycloalkenyl, C₆₋₁₄ aryl or heterocycle R² may be fused to 5-7 membered carbocyclic or heterocyclic rings;
R³is hydrogen, hydroxy, C₁₋₈ alkyl, C₁₋₈ haloalkyl, C₃₋₇ cycloalkyl, C₂₋₆ alkenyl, C₃₋₇ cycloalkenyl, C₃₋₆ alkynyl, N(R^{a}R^{b}), or heterocycle, each of which may be optionally substituted with one or more substituents independently selected from the group consisting of C₁₋₈ alkyl, C₁₋₈ haloalkyl, C₃₋₇ cycloalkyl, C₂₋₆ alkenyl, C₃₋₇ cycloalkenyl, C₃₋₆ alkynyl, halogen, oxo, CN, NO₂, OR^{a}, N(R^{a}R^{b}), S(O)ₘR^{a}, SR^{a}, OS(O)ₘR^{a}, S(O)ₘOR^{a}, OS(O)ₘOR^{a}, N(R^{a})S(O)ₘR^{b}, S(O)ₘN(R^{a}R^{b}), N(R^{a})S(O)ₘN(R^{a}R^{b}), OS(O)ₘN(R^{a}R^{b}), N(R^{a})S(O)ₘOR^{b}, C(O)R^{a}, OC(O)R^{a}, C(O)OR^{a}, OC(O)OR^{a}, N(R^{a})C(O)R^{b}, C(O)N(R^{a}R^{b}), N(R^{a})C(O)N(R^{a}R^{b}), OC(O)N(R^{a}R^{b}), N(R^{a})C(O)OR^{b}, C(NR^{a})═N(R^{b}), C(SR^{a})═N(R^{b}), C(OR^{a})═N(R^{b}), N(R^{a})C(NR^{a}R^{b})═N(R^{a}), N(R^{a})C(SR^{a})═N(R^{b}), N(R^{a})C(OR^{a})═N(R^{b}), and heterocycle optionally substituted by oxo or R^{a};
R^{a} and R^{b} are independently hydrogen, NO₂, OR^{c}, CN, N(R^{c}R^{d}), C(O)R^{c}, C(O)C(O)R^{c}, C(O)N(R^{c}R^{d}), C(O)C(O)N(R^{c}R^{d}), S(O)ₘR^{c}, SR^{c}, S(O)ₘN(R^{c}R^{d}), C₁₋₈ alkyl, C₁₋₈ haloalkyl, C₃₋₇ cycloalkyl, C₆₋₁₄ aralkyl, C₂₋₆ alkenyl, C₃₋₇ cycloalkenyl, C₃₋₆ alkynyl, C₆₋₁₄ aryl or heterocycle, each of which may be optionally substituted with one or more substituents independently selected from the group consisting of C₁₋₈ alkyl, C₁₋₈ haloalkyl, C₃₋₇ cycloalkyl, C₆₋₁₄ aralkyl, C₂₋₆ alkenyl, C₃₋₇ cycloalkenyl, C₃₋₆ alkynyl, C₆₋₁₄ aryl, CN, NO₂, OR^{c}, N(R^{c}R^{d}), S(O)ₘR^{c}, SR^{c}, OS(O)ₘR^{c}, S(O)ₘOR^{c}, OS(O)ₘOR^{c}, N(R^{c})S(O)ₘR^{d}, S(O)ₘN(R^{c}R^{d}), N(R^{c})S(O)ₘN(R^{c}R^{d}), OS(O)ₘN(R^{c}R^{d}), N(R^{c})S(O)ₘOR^{d}, C(O)R^{c}, OC(O)R^{c}, C(O)OR^{c}, OC(O)OR^{c}, N(R^{c})C(O)R^{d}, C(O)N(R^{c}R^{d}), N(R^{c})C(O)N(R^{c}R^{d}), OC(O)N(R^{c}R^{d}), N(R^{c})C(O)OR^{d}, C(NR^{c}R^{d})═N(R^{c}), C(SR^{c})═N(R^{d}), C(OR^{c})═N(R^{d}) and heterocycle;
Optionally, R^{a} and R^{b} may be linked together through one or more ring carbon atoms and/or ring heteroatoms including N, O, C(R^{c}R^{d}), C(O), S(O)ₘ, or S to form a saturated or unsaturated 3 to 8 membered carbocyclic or heterocyclic ring optionally substituted with oxo or R^{a};
R^{c} and R^{d} are independently hydrogen, C₁₋₈ alkyl, C₁₋₈ haloalkyl, C₃₋₇ cycloalkyl, C₆₋₁₄ aralkyl, C₂₋₆ alkenyl, C₃₋₇ cycloalkenyl, C₃₋₆ alkynyl, C₆₋₁₄ aryl or heterocycle;
Optionally, R^{c} and R^{d} may be linked together through one or more ring carbon atoms and/or ring heteroatoms including N, O, C(O) and S(O)ₘ, or S to form a saturated or unsaturated 3 to 8 membered carbocyclic or heterocyclic ring;
m is 1 or 2;
   or a pharmaceutically acceptable salt thereof.

### DETAILED DESCRIPTION OF THE INVENTION

The present invention includes the compounds of formula (I), useful in treating or preventing viral infections, particularly HIV infections, pharmaceutical compositions comprising compounds of formula (I), and processes for preparing the compounds.

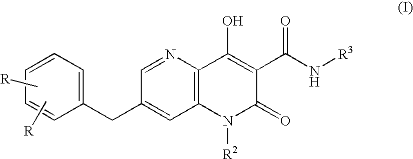

wherein:
R is halogen;
R¹ is
(a) C(O)N(R^{a}R^{b});
(b) C₃₋₆ alkynyl or C₁₋₈ alkylcarbonyl, each of which may be optionally substituted with one or more substituents independently selected from the group consisting of C₁₋₈ alkyl, C₁₋₈ haloalkyl, C₃₋₇ cycloalkyl, C₂₋₆ alkenyl, C₃₋₇ cycloalkenyl, C₃₋₆ alkynyl, halogen, CN, NO₂, OR^{a}, N(R^{a}R^{b}), S(O)ₘR^{a}, SR^{a}, OS(O)ₘR^{a}, S(O)ₘOR^{a}, OS(O)ₘOR^{a}, N(R^{a})S(O)ₘR^{b}, S(O)ₘN(R^{a}R^{b}), N(R^{a})S(O)ₘN(R^{a}R^{b}), OS(O)ₘN(R^{a}R^{b}), N(R^{a})S(O)ₘOR^{b}, C(O)R^{a}, OC(O)R^{a}, C(O)OR^{a}, OC(O)OR^{a}, N(R^{a})C(O)R^{b}, C(O)N(R^{a}R^{b}), N(R^{a})C(O)N(R^{a}R^{b}), OC(O)N(R^{a}R^{b}), N(R^{a})C(O)OR^{b}, C(NR^{a}R^{b})═N(R^{a}), N(R^{a})C(NR^{a}R^{b})═N(R^{a}), C(SR^{a})═N(R^{b}), C(OR^{a})═N(R^{b}), N(R^{a})C(SR^{a})═N(R^{b}) and heterocycle optionally substituted with oxo or R^{a}; or
(c) C₁₋₈ alkyl or C₁₋₈ alkoxy, each of which is substituted with one or more substituents independently selected from the group consisting of C₆₋₁₄ aryl, C₁₋₈ alkyl, C₁₋₈ haloalkyl, C₃₋₇ cycloalkyl, C₂₋₆ alkenyl, C₃₋₇ cycloalkenyl, C₃₋₆ alkynyl, halogen, CN, NO₂, OR^{a}, N(R^{a}R^{b}), S(O)ₘR^{a}, SR^{a}, OS(O)ₘR^{a}, S(O)ₘOR^{a}, OS(O)ₘOR^{a}, N(R^{a})S(O)ₘR^{b}, S(O)ₘN(R^{a}R^{b}), N(R^{a})S(O)ₘN(R^{a}R^{b}), OS(O)ₘN(R^{a}R^{b}), N(R^{a})S(O)ₘOR^{b}, C(O)R^{a}, OC(O)R^{a}, C(O)OR^{a}, OC(O)OR^{a}, N(R^{a})C(O)R^{b}, C(O)N(R^{a}R^{b}), N(R^{a})C(O)N(R^{a}R^{b}), OC(O)N(R^{a}R^{b}), N(R^{a})C(O)OR^{b}, C(NR^{a}R^{b})═N(R^{a}), N(R^{a})C(NR^{a}R^{b})═N(R^{a}), C(SR^{a})═N(R^{b}), C(OR^{a})═N(R^{b}), N(R^{a})C(SR^{a})═N(R^{b}) and heterocycle optionally substituted with oxo or R^{a},
R² is hydrogen, C₁₋₈ alkyl, C₁₋₈ haloalkyl, C₃₋₇ cycloalkyl, C₆₋₁₄ aralkyl, C₂₋₆ alkenyl, C₃₋₇ cycloalkenyl, C₃₋₆ alkynyl, C₆₋₁₄ aryl, or heterocycle, each of which may be optionally substituted with one or more substituents independently selected from the group consisting of C₁₋₈ alkyl, C₁₋₈ haloalkyl, C₃₋₇ cycloalkyl, C₂₋₆ alkenyl, C₃₋₇ cycloalkenyl, C₃₋₆ alkynyl, halogen, CN, NO₂, OR^{a}, N(R^{a}R^{b}), S(O)ₘR^{a}, SR^{a}, OS(O)ₘR^{a}, S(O)ₘOR^{a}, OS(O)ₘOR^{a}, N(R^{a})S(O)ₘR^{b}, S(O)ₘN(R^{a}R^{b}), N(R^{a})S(O)ₘN(R^{a}R^{b}), OS(O)ₘN(R^{a}R^{b}), N(R^{a})S(O)ₘOR^{b}, C(O)R^{a}, OC(O)R^{a}, C(O)OR^{a}, OC(O)OR^{a}, N(R^{a})C(O)R^{b}, C(O)N(R^{a}R^{b}), N(R^{a})C(O)N(R^{a}R^{b}), OC(O)N(R^{a}R^{b}), N(R^{a})C(O)OR^{b}, C(NR^{a}R^{b})═N(R^{a}), N(R^{a})C(NR^{a}R^{b})═N(R^{a}), C(SR^{a})═N(R^{b}), C(OR^{a})═N(R^{b}), N(R^{a})C(SR^{a})═N(R^{b}) and heterocycle optionally substituted with oxo or R^{a};
or optionally when R² is C₅₋₇ cycloalkyl, C₆₋₁₄ aralkyl, C₅₋₇ cycloalkenyl, C₆₋₁₄ aryl or heterocycle R² may be fused to 5-7 membered carbocyclic or heterocyclic rings;
R³ is hydrogen, hydroxy, C₁₋₈ alkyl, C₁₋₈ haloalkyl, C₃₋₇ cycloalkyl, C₂₋₆ alkenyl, C₃₋₇ cycloalkenyl, C₃₋₆ alkynyl, N(R^{a}R^{b}), or heterocycle, each of which may be optionally substituted with one or more substituents independently selected from the group consisting of C₁₋₈ alkyl, C₁₋₈ haloalkyl, C₃₋₇ cycloalkyl, C₂₋₆ alkenyl, C₃₋₇ cycloalkenyl, C₃₋₆ alkynyl, halogen, oxo, CN, NO₂, OR^{a}, N(R^{a}R^{b}), S(O)ₘR^{a}, SR^{a}, OS(O)ₘR^{a}, S(O)ₘOR^{a}, OS(O)ₘOR^{a}, N(R^{a})S(O)ₘR^{b}, S(O)ₘN(R^{a}R^{b}), N(R^{a})S(O)ₘN(R^{a}R^{b}), OS(O)ₘN(R^{a}R^{b}), N(R^{a})S(O)ₘOR^{b}, C(O)R^{a}, OC(O)R^{a}, C(O)OR^{a}, OC(O)OR^{a}, N(R^{a})C(O)R^{b}, C(O)N(R^{a}R^{b}), N(R^{a})C(O)N(R^{a}R^{b}), OC(O)N(R^{a}R^{b}), N(R^{a})C(O)OR^{b}, C(NR^{a})═N(R^{b}), C(SR^{a})═N(R^{b}), C(OR^{a})═N(R^{b}), N(R^{a})C(NR^{a}R^{b})═N(R^{a}), N(R^{a})C(SR^{a})═N(R^{b}), N(R^{a})C(OR^{a})═N(R^{b}), and heterocycle optionally substituted by oxo or R^{a};
R^{a} and R^{b} are independently hydrogen, NO₂, OR^{c}, CN, N(R^{c}R^{d}), C(O)R^{c}, C(O)C(O)R^{c}, C(O)N(R^{c}R^{d}), C(O)C(O)N(R^{c}R^{d}), S(O)ₘR^{c}, SR^{c}, S(O)ₘN(R^{c}R^{d}), C₁₋₈ alkyl, C₁₋₈ haloalkyl, C₃₋₇ cycloalkyl, C₆₋₁₄ aralkyl, C₂₋₆ alkenyl, C₃₋₇ cycloalkenyl, C₃₋₆ alkynyl, C₆₋₁₄ aryl or heterocycle, each of which may be optionally substituted with one or more substituents independently selected from the group consisting of C₁₋₈ alkyl, C₁₋₈ haloalkyl, C₃₋₇ cycloalkyl, C₆₋₁₄ aralkyl, C₂₋₆ alkenyl, C₃₋₇ cycloalkenyl, C₃₋₆ alkynyl, C₆₋₁₄ aryl, CN, NO₂, OR^{c}, N(R^{c}R^{d}), S(O)ₘR^{c}, SR^{c}, OS(O)ₘR^{c}, S(O)ₘOR^{c}, OS(O)ₘOR^{c}, N(R^{c})S(O)ₘR^{d}, S(O)ₘN(R^{c}R^{d}), N(R^{c})S(O)ₘN(R^{c}R^{d}), OS(O)ₘN(R^{c}R^{d}), N(R^{c})S(O)ₘOR^{d}, C(O)R^{c}, OC(O)R^{c}, C(O)OR^{c}, OC(O)OR^{c}, N(R^{c})C(O)R^{d}, C(O)N(R^{c}R^{d}), N(R^{c})C(O)N(R^{c}R^{d}), OC(O)N(R^{c}R^{d}), N(R^{c})C(O)OR^{d}, C(NR^{c}R^{d})═N(R^{c}), C(SR^{c})═N(R^{d}), C(OR^{c})═N(R^{d}) and heterocycle;
Optionally, R^{a} and R^{b} may be linked together through one or more ring carbon atoms and/or ring heteroatoms including N, O, C(R^{c}R^{d}), C(O), S(O)ₘ, or S to form a saturated or unsaturated 3 to 8 membered carbocyclic or heterocyclic ring optionally substituted with oxo or R^{a};
R^{c} and R^{d} are independently hydrogen, C₁₋₈ alkyl, C₁₋₈ haloalkyl, C₃₋₇ cycloalkyl, C₆₋₁₄ aralkyl, C₂₋₆ alkenyl, C₃₋₇ cycloalkenyl, C₃₋₆ alkynyl, C₆₋₁₄ aryl or heterocycle;
Optionally, R^{c} and R^{d} may be linked together through one or more ring carbon atoms and/or ring heteroatoms including N, O, C(O) and S(O)ₘ, or S to form a saturated or unsaturated 3 to 8 membered carbocyclic or heterocyclic ring;
m is 1 or 2;
   or a pharmaceutically acceptable salt thereof.

The term “alkyl”, alone or in combination with any other term, refers to a straight-chain or branched-chain saturated aliphatic hydrocarbon radical containing the specified number of carbon atoms. Examples of alkyl radicals include, but are not limited to, methyl, ethyl, n-propyl, isopropyl, n-butyl, isobutyl, sec-butyl, tert-butyl, pentyl, isoamyl, n-hexyl and the like.

The term “cycloalkyl” refers to a saturated or partially saturated carbocyclic ring composed of 3-6 carbons in any chemically stable configuration. Examples of suitable carbocyclic groups include cyclopropyl, cyclobutyl, cyclopentyl, cyclohexyl, and cyclohexenyl.

The term “alkenyl,” alone or in combination with any other term, refers to a straight-chain or branched-chain alkyl group with at least one carbon-carbon double bond. Examples of alkenyl radicals include, but are not limited to, ethenyl, propenyl, isopropenyl, butenyl, isobutenyl, pentenyl, hexenyl, hexadienyl and the like.

The term “alkynyl” refers to hydrocarbon groups of either a straight or branched configuration with one or more carbon-carbon triple bonds which may occur in any stable point along the chain, such as ethynyl, propynyl, butynyl, pentynyl, and the like.

The term “alkoxy” refers to an alkyl ether radical, wherein the term “alkyl” is defined above. Examples of suitable alkyl ether radicals include, but are not limited to, methoxy, ethoxy, n-propoxy, isopropoxy, n-butoxy, isobutoxy, sec-butoxy, tert-butoxy and the like.

The term “aryl” alone or in combination with any other term, refers to a carbocyclic aromatic moiety (such as phenyl or naphthyl) containing the specified number of carbon atoms, preferably from 6-14 carbon atoms, and more preferably from 6-10 carbon atoms. Examples of aryl radicals include, but are not limited to, phenyl, naphthyl, indenyl, azulenyl, fluorenyl, anthracenyl, phenanthrenyl, tetrahydronaphthyl, indanyl, phenanthridinyl and the like. Unless otherwise indicated, the term “aryl” also includes each possible positional isomer of an aromatic hydrocarbon radical, such as in 1-naphthyl, 2-naphthyl, 5-tetrahydronaphthyl, 6-tetrahydronaphthyl, 1-phenanthridinyl, 2-phenanthridinyl, 3-phenanthridinyl, 4-phenanthridinyl, 7-phenanthridinyl, 8-phenanthridinyl, 9-phenanthridinyl and 10-phenanthridinyl. Examples of aryl radicals include, but are not limited to, phenyl, naphthyl, indenyl, azulenyl, fluorenyl, anthracenyl, phenanthrenyl, tetrahydronaphthyl, indanyl, phenanthridinyl and the like.

The term “aralkyl” refers to an alkyl group substituted by an aryl group. Examples of aralkyl groups include, but are not limited to, benzyl, phenethyl and the like.

The term “heterocycle,” “heterocyclic,” and “heterocyclyl” as used herein, refer to a 3- to 7-membered monocyclic heterocyclic ring or 8- to 11-membered bicyclic heterocyclic ring system any ring of which is either saturated, partially saturated or unsaturated, and which may be optionally benzofused if monocyclic. Each heterocycle consists of one or more carbon atoms and from one to four heteroatoms selected from the group consisting of N, O and S, and wherein the nitrogen and sulfur heteroatoms may optionally be oxidized, and the nitrogen atom may optionally be quaternized, and including any bicyclic group in which any of the above-defined heterocyclic rings is fused to a benzene ring. The heterocyclic ring may be attached at any carbon or heteroatom, provided that the attachment results in the creation of a stable structure. Preferred heterocycles include 5-7 membered monocyclic heterocycles and 8-10 membered bicyclic heterocycles. When the heterocyclic ring has substituents, it is understood that the substituents may be attached to any atom in the ring, whether a heteroatom or a carbon atom, provided that a stable chemical structure results. “Heteroaromatics” or “heteroaryl” are included within the heterocycles as defined above and generally refers to a heterocycle in which the ring system is an aromatic monocyclic or polycyclic ring radical containing five to twenty carbon atoms, preferably five to ten carbon atoms, in which one or more ring carbons, preferably one to four, are each replaced by a heteroatom such as N, O, S and P. Preferred heteroaryl groups include 5-6 membered monocyclic heteroaryls and 8-10 membered bicyclic heteroaryls. Also included within the scope of the term “heterocycle, “heterocyclic” or “heterocyclyl” is a group in which a non-aromatic heteroatom-containing ring is fused to one or more aromatic rings, such as in an indolinyl, chromanyl, phenanthridinyl or tetrahydro-quinolinyl, where the radical or point of attachment is on the non-aromatic heteroatom-containing ring. Unless otherwise indicated, the term “heterocycle, “heterocyclic” or “heterocyclyl” also included each possible positional isomer of a heterocyclic radical, such as in 1-indolinyl, 2-indolinyl, 3-indolinyl. Examples of heterocycles include imidazolyl, imidazolinoyl, imidazolidinyl, quinolyl, isoquinolyl, indolyl, indazolyl, indazolinolyl, perhydropyridazyl, pyridazyl, pyridyl, pyrrolyl, pyrrolinyl, pyrrolidinyl, pyrazolyl, pyrazinyl, quinoxolyl, piperidinyl, pyranyl, pyrazolinyl, piperazinyl, pyrimidinyl, pyridazinyl, morpholinyl, thiamorpholinyl, furyl, thienyl, triazolyl, thiazolyl, carbolinyl, tetrazolyl, thiazolidinyl, benzofliranoyl, thiamorpholinyl sulfone, oxazolyl, oxadiazolyl, benzoxazolyl, oxopiperidinyl, oxopyrrolidinyl, oxoazepinyl, azepinyl, isoxozolyl, isothiazolyl, furazanyl, tetrahydropyranyl, tektahydrofuranyl, thiazolyl, thiadiazoyl, dioxolyl, dioxinyl, oxathiolyl, benzodioxolyl, dithiolyl, thiophenyl, tetrahydrothiophenyl, sulfolanyl, dioxanyl, dioxolanyl, tetahydroffirodihydrofuranyl, tetrahydropyranodihydrofuranyl, dihydropyranyl, tetradyrofurofuranyl and tetrahydropyranofuranyl.

The term “heteroatom” means nitrogen, oxygen, or sulfur and includes any oxidized form of nitrogen, such as N(O){N⁺—O⁻} and sulfur such as S(O) and S(O)₂, and the quaternized form of any basic nitrogen.

A combination of substituents or variables is permissible only if such a combination results in a stable or chemically feasible compound. A stable compound or chemically feasible compound is one in which the chemical structure is not substantially altered when kept at a temperature of 40° C. or less, in the absence of moisture or other chemically reactive conditions, for at least a week.

Unless otherwise stated, structures depicted herein are also meant to include all stereochemical forms of the structure, i.e., the R and S configurations for each asymmetric center. Therefore, racemates and racemic mixtures, single enantiomers, diastereomeric mixtures and individual diastereoisomers of the present compounds are expressly included within the scope of the invention. Although the specific compounds exemplified herein may be depicted in a particular stereochemical configuration, compounds having either the opposite stereochemistry at any given chiral center or mixtures thereof are also envisioned.

Unless otherwise stated, structures depicted herein are also meant to include compounds which differ only in the presence of one or more isotopically enriched atoms. For example, compounds having the present structures except for the replacement of a hydrogen by a deuterium or tritium, or the replacement of a carbon by a ¹³C- or ¹⁴C-enriched carbon are also within the scope of this invention.

It will be apparent to one skilled in the art that certain compounds of this invention may exist in alternative tautomeric forms. All such tautomeric forms of the present compounds are within the scope of the invention. Unless otherwise indicated, the representation of either tautomer is meant to include the other.

The term “pharmaceutically effective amount” refers to an amount effective in treating a virus infection, for example an HIV infection, in a patient either as monotherapy or in combination with other agents. The term “treating” as used herein refers to the alleviation of symptoms of a particular disorder in a patient, or the improvement of an ascertainable measurement associated with a particular disorder, and may include the suppression of symptom recurrence in an asymptomatic patient such as a patient in whom a viral infection has become latent. The term “prophylactically effective amount” refers to an amount effective in preventing a virus infection, for example an HIV infection, or preventing the occurrence of symptoms of such an infection, in a patient. As used herein, the term “patient” refers to a mammal, including a human.

The term “pharmaceutically acceptable carrier or adjuvant” refers to a carrier or adjuvant that may be administered to a patient, together with a compound of this invention, and which does not destroy the pharmacological activity thereof and is nontoxic when administered in doses sufficient to deliver a therapeutic amount of the antiviral agent.

The term “treatment” as used herein refers to the alleviation of symptoms of a particular disorder in a patient, or the improvement of an ascertainable measurement associated with a particular disorder, and may include the suppression of symptom recurrence in an asymptomatic patient such as a patient in whom a viral infection has become latent. Treatment includes prophylaxis which refers to preventing a disease or condition or preventing the occurrence of symptoms of such a disease or condition, in a patient. As used herein, the term “patient” refers to a mammal, including a human.

As used herein, the term “subject” refers to a patient, animal or a biological sample. The term “biological sample”, as used herein, includes, without limitation, cell cultures or extracts thereof; preparations of an enzyme suitable for in vitro assay; biopsied material obtained from a mammal or extracts thereof; and blood, saliva, urine, feces, semen, tears, or other body fluids or extracts thereof.

Throughout this specification, the word “comprise” or variations such as “comprises” or “comprising” will be understood to imply the inclusion of a stated integer or groups of integers but not the exclusion of any other integer or group of integers.

As used herein, the compounds according to the invention are defined to include pharmaceutically acceptable derivatives thereof. A “pharmaceutically acceptable derivative” means any pharmaceutically acceptable salt, ester, salt of an ester, ether, or other derivative of a compound of this invention which, upon administration to a recipient, is capable of providing directly or indirectly a compound of this invention or an inhibitorily active metabolite or residue thereof. Particularly favored derivatives and prodrugs are those that increase the bioavailability of the compounds of this invention when such compounds are administered to a mammal, for example, by allowing an orally administered compound to be more readily absorbed into the blood, or which enhance delivery of the parent compound to a biological compartment, for example, the brain or lymphatic system, relative to the parent species.

Pharmaceutically acceptable salts of the compounds according to the invention include those derived from pharmaceutically acceptable inorganic and organic acids and bases. Examples of suitable acids include hydrochloric, hydrobromic, sulfuric, nitric, perchloric, fumaric, maleic, phosphoric, glycollic, lactic, salicyclic, succinic, toluene-p-sulfonic, tartaric, acetic, citric, methanesulfonic, ethanesulfonic, formic, benzoic, malonic, naphthalene-2-sulfonic and benzenesulfonic acids. Other acids, such as oxalic, while not in themselves pharmaceutically acceptable, may be employed in the preparation of salts useful as intermediates in obtaining the compounds of the invention and their pharmaceutically acceptable acid addition salts.

Salts derived from appropriate bases include alkali metal (e.g. sodium), alkaline earth metal (e.g., magnesium), ammonium, NW₄⁺ (wherein W is C₁₋₄ alkyl) and other amine salts. Physiologically acceptable salts of a hydrogen atom or an amino group include salts or organic carboxylic acids such as acetic, lactic, tartaric, malic, isethionic, lactobionic and succinic acids; organic sulfonic acids such as methanesulfonic, ethanesulfonic, benzenesulfonic and p-toluenesulfonic acids and inorganic acids such as hydrochloric, sulfuric, phosphoric and sulfamic acids. Physiologically acceptable salts of a compound with a hydroxy group include the anion of said compound in combination with a suitable cation such as Na⁺, NH₄⁺, and NW₄⁺ (wherein W is a C₁₋₄alkyl group). Preferred salts include sodium, calcium, potassium, magnesium, choline, meglumine, hydrochloride, and quaternary ammonium.

Other compounds of this invention may be prepared by one skilled in the art following the teachings of the specification coupled with knowledge in the art using reagents that are readily synthesized or commercially available.

Any reference to any of the above compounds also includes a reference to a pharmaceutically acceptable salt thereof.

Salts of the compounds of the present invention may be made by methods known to a person skilled in the art. For example, treatment of a compound of the present invention with an appropriate base or acid in an appropriate solvent will yield the corresponding salt.

Esters of the compounds of the present invention are independently selected from the following groups: (1) carboxylic acid esters obtained by esterification of the hydroxy groups, in which the non-carbonyl moiety of the carboxylic acid portion of the ester grouping is selected from straight or branched chain alkyl (for example, acetyl, n-propyl, t-butyl, or n-butyl), alkoxyalkyl (for example, methoxymethyl), aralkyl (for example, benzyl), aryloxyalkyl (for example, phenoxymethyl), aryl (for example, phenyl optionally substituted by, for example, halogen, C₁₋₄alkyl, or C₁₋₄alkoxy or amino); (2) sulfonate esters, such as alkyl- or aralkylsulfonyl (for example, methanesulfonyl); (3) amino acid esters (for example, L-valyl or L-isoleucyl); (4) phosphonate esters and (5) mono-, di- or triphosphate esters. The phosphate esters may be further esterified by, for example, a C₁₋₂₀ alcohol or reactive derivative thereof, or by a 2,3-di(C₆₋₂₄)acyl glycerol.

In such esters, unless otherwise specified, any alkyl moiety present advantageously contains from 1 to 18 carbon atoms, particularly from 1 to 6 carbon atoms, more particularly from 1 to 4 carbon atoms, Any cycloalkyl moiety present in such esters advantageously contains from 3 to 6 carbon atoms. Any aryl moiety present in such esters advantageously comprises a phenyl group.

Ethers of the compounds of the present invention include, but are not limited to methyl, ethyl, butyl and the like.

The present invention features a compound of formula (I) wherein:
R is halogen;
R¹ is
(a) C₁₋₈alkoxy substituted with C₆₋₁₄aryl; OR^{a}; or NR^{a}R^{b};
(b) C(O)NR^{a}R^{b};
(c) C₃₋₆alkynyl substituted with OR^{a} or NR^{a}R^{b};
(d) C₁₋₈alkylcarbonyl; or
(e) C₁₋₈alkyl substituted with OR^{a}; C(O)NR^{a}R^{b}; N(R^{a})S(O)ₘR^{b}; N(R^{a})C(O)R^{b}; NR^{a}R^{b}; or heterocycle optionally substituted with one or more oxo;
R² is C₁₋₈alkyl;
R³ is
(a) C₁₋₈alkyl optionally substituted with C₁₋₈alkyl, C₃₋₇cycloalkyl, OR^{a}, SR^{a}, C(O)N(R^{a}R^{b}), NR^{a}C(O)R^{b}, or heterocycle optionally substituted with oxo or R^{a};
(b) C₃₋₇cycloalkyl;
(c) C₁₋₈haloalkyl; or
(d) heterocycle optionally substituted with oxo;
R^{a} and R^{b} are independently hydrogen, NO₂, OR^{c}, CN, N(R^{c}R^{d}), C(O)R^{c}, C(O)C(O)R^{c}, C(O)N(R^{c}R^{d}), C(O)C(O)N(R^{c}R^{d}), S(O)ₘR^{c}, SR^{c}, S(O)ₘN(R^{c}R^{d}), C₁₋₈ alkyl, C₁₋₈ haloalkyl, C₃₋₇ cycloalkyl, C₆₋₁₄ aralkyl, C₂₋₆ alkenyl, C₃₋₇ cycloalkenyl, C₃₋₆ alkynyl, C₆₋₁₄ aryl or heterocycle, each of which may be optionally substituted with one or more substituents independently selected from the group consisting of C₁₋₈ alkyl, C₁₋₈ haloalkyl, C₃₋₇ cycloalkyl, C₆₋₁₄ aralkyl, C₂₋₆ alkenyl, C₃₋₇ cycloalkenyl, C₃₋₆ alkynyl, C₆₋₁₄ aryl, CN, NO₂, OR^{c}, N(R^{c}R^{d}), S(O)ₘR^{c}, SR^{c}, OS(O)ₘR^{c}, S(O)ₘOR^{c}, OS(O)ₘOR^{c}, N(R^{c})S(O)ₘR^{d}, S(O)ₘN(R^{c}R^{d}), N(R^{c})S(O)ₘN(R^{c}R^{d}), OS(O)ₘN(R^{c}R^{d}), N(R^{c})S(O)ₘOR^{d}, C(O)R^{c}, OC(O)R^{c}, C(O)OR^{c}, OC(O)OR^{c}, N(R^{c})C(O)R^{d}, C(O)N(R^{c}R^{d}), N(R^{c})C(O)N(R^{c}R^{d}), OC(O)N(R^{c}R^{d}), N(R^{c})C(O)OR^{d}, C(NR^{c}R^{d})═N(R^{c}), C(SR^{c})═N(R^{d}), C(OR^{c})═N(R^{d}) and heterocycle;
Optionally, R^{a} and R^{b} may be linked together through one or more ring carbon atoms and/or ring heteroatoms including N, O, C(R^{c}R^{d}), C(O), S(O)ₘ, or S to form a saturated or unsaturated 3 to 8 membered carbocyclic or heterocyclic ring optionally substituted with oxo or R^{a};
R^{c} and R^{d} are independently hydrogen, C₁₋₈ alkyl, C₁₋₈ haloalkyl, C₃₋₇ cycloalkyl, C₆₋₁₄ aralkyl, C₂₋₆ alkenyl, C₃₋₇ cycloalkenyl, C₃₋₆ alkynyl, C₆₋₁₄ aryl or heterocycle;
Optionally, R^{c} and R^{d} may be linked together through one or more ring carbon atoms and/or ring heteroatoms including N, O, C(O) and S(O)ₘ, or S to form a saturated or unsaturated 3 to 8 membered carbocyclic or heterocyclic ring;
m is 1 or 2;
   or a pharmaceutically acceptable salt thereof.

The present invention features a compound of formula (I) wherein:
R is halogen;
R¹ is
(a) C₁₋₈alkoxy substituted with C₆₋₁₄aryl; OR^{a} (wherein R^{a} is C₁₋₈alkyl); or NR^{a}R^{b} (wherein R^{a} and R^{b} are independently hydrogen or C(O)R^{c} wherein R^{c} is C₁₋₈alkyl);
(b) C(O)NR^{a} R^{b} wherein R^{a} and R^{b} are independently hydrogen, C₁₋₈ alkyl, or C(O)R^{c} wherein R^{c} is C₁₋₈alkyl;
(c) C₃₋₆alkynyl substituted with OR^{a} (wherein R^{a} is C₁₋₈alkyl) or NR^{a}R^{b} (wherein R^{a} and R^{b} are independently hydrogen or C(O)R^{c} wherein R^{c} is C₁₋₈alkyl);
(d) C₁₋₈alkylcarbonyl; or
(e) C₁₋₈alkyl substituted with OR^{a} (wherein R^{a} is hydrogen or C(O)N(R^{c}R^{d}) wherein R^{c} and R^{d} are independently hydrogen or C₁₋₈alkyl); C(O)NR^{a}R^{b} (wherein R^{a} and R^{b} are hydrogen, C₁₋₈alkyl, CN or OR^{c} wherein R^{c} is hydrogen); N(R^{a})S(O)ₘR^{b} (wherein R^{a} and R^{b} are independently hydrogen or C₁₋₈alkyl and m is 2); N(R^{a})C(O)R^{b} (wherein R^{a} and R^{b} are C₁₋₈alkyl); NR^{a} R^{b} (wherein R^{a} and R^{b} are independently hydrogen, C₁₋₈alkyl or C(O)R^{c} wherein R^{c} is C₁₋₈alkyl); or heterocycle optionally substituted with one or more oxo;
R² is C₁₋₈alkyl;
R³ is C₁₋₈alkyl optionally substituted with OR^{a} (wherein R^{a} is hydrogen or C₁₋₈alkyl);
   or a pharmaceutically acceptable salt thereof.

The present invention also features a compound of formula (I) wherein
R is halogen;
R¹ is C₁₋₈alkoxy substituted with C₆₋₁₄aryl; OR^{a} (wherein R^{a} is C₁₋₈alkyl); or NR^{a}R^{b} (wherein R^{a} and R^{b} are independently hydrogen or C(O)R^{c} wherein R^{c} is C₁₋₈alkyl);
R² is C₁₋₈alkyl;
R³ is C₁₋₈alkyl optionally substituted with OR^{a} (wherein R^{a} is hydrogen or C₁₋₈alkyl);
   or a pharmaceutically acceptable salt thereof.

The present invention features a compound of formula (Ia)

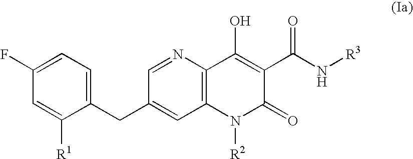

wherein:
R¹ is
(a) C(O)N(R^{a}R^{b}),
(b) C₃₋₆ alkynyl or C₁₋₈ alkylcarbonyl, each of which may be optionally substituted with one or more substituents independently selected from the group consisting of C₁₋₈ alkyl, C₁₋₈ haloalkyl, C₃₋₇ cycloalkyl, C₂₋₆ alkenyl, C₃₋₇ cycloalkenyl, C₃₋₆ alkynyl, halogen, CN, NO₂, OR^{a}, NR^{a}R^{b}), S(O)ₘR^{a}, SR^{a}, OS(O)ₘR^{a}, S(O)ₘOR^{a}, OS(O)ₘOR^{a}, N(R^{a})S(O)ₘR^{b}, S(O)ₘN(R^{a}R^{b}), N(R^{a})S(O)ₘN(R^{a}R^{b}), OS(O)ₘN(R^{a}R^{b}), N(R^{a})S(O)ₘOR^{b}, C(O)R^{a}, OC(O)R^{a}, C(O)OR^{a}, OC(O)OR^{a}, N(R^{a})C(O)R^{b}, C(O)N(R^{a}R^{b}), N(R^{a})C(O)N(R^{a}R^{b}), OC(O)N(R^{a}R^{b}), N(R^{a})C(O)OR^{b}, C(NR^{a}R^{b})═N(R^{a}), N(R^{a})C(NR^{a}R^{b})═N(R^{a}), C(SR^{a})═N(R^{b}), C(OR^{a})═N(R^{b}), N(R^{a})C(SR^{a})═N(R^{b}) and heterocycle optionally substituted with oxo or R^{a}, or
(c) C₁₋₈ alkyl or C₁₋₈ alkoxy, each of which is substituted with one or more substituents independently selected from the group consisting of C₆₋₁₄ aryl, C₁₋₈ alkyl, C₁₋₈ haloalkyl, C₃₋₇ cycloalkyl, C₂₋₆ alkenyl, C₃₋₇ cycloalkenyl, C₃₋₆ alkynyl, halogen, CN, NO₂, OR^{a}, N(R^{a}R^{b}), S(O)ₘR^{a}, SR^{a}, OS(O)ₘR^{a}, S(O)ₘOR^{a}, OS(O)ₘOR^{a}, N(R^{a})S(O)ₘR^{b}, S(O)ₘN(R^{a}R^{b}), N(R^{a})S(O)ₘN(R^{a}R^{b}), OS(O)ₘN(R^{a}R^{b}), N(R^{a})S(O)ₘOR^{b}, C(O)R^{a}, OC(O)R^{a}, C(O)OR^{a}, OC(O)OR^{a}, N(R^{a})C(O)R^{b}, C(O)N(R^{a}R^{b}), N(R^{a})C(O)N(R^{a}R^{b}), OC(O)N(R^{a}R^{b}), N(R^{a})C(O)OR^{b}, C(NR^{a}R^{b})═N(R^{a}), N(R^{a})C(NR^{a}R^{b})═N(R^{a}), C(SR^{a})═N(R^{b}), C(OR^{a})═N(R^{b}), N(R^{a})C(SR^{a})═N(R^{b}) and heterocycle optionally substituted with oxo or R^{a};
R² is hydrogen, C₁₋₈ alkyl, C₁₋₈ haloalkyl, C₃₋₇ cycloalkyl, C₆₋₁₄ aralkyl, C₂₋₆ alkenyl, C₃₋₇ cycloalkenyl, C₃₋₆ alkynyl, C₆₋₁₄ aryl or heterocycle, each of which may be optionally substituted with one or more substituents independently selected from the group consisting of C₁₋₈ alkyl, C₁₋₈ haloalkyl, C₃₋₇ cycloalkyl, C₂₋₆ alkenyl, C₃₋₇ cycloalkenyl, C₃₋₆ alkynyl, halogen, CN, NO₂, OR^{a}, N(R^{a}R^{b}), S(O)ₘR^{a}, SR^{a}, OS(O)ₘR^{a}, S(O)ₘOR^{a}, OS(O)ₘOR^{a}, N(R^{a})S(O)ₘR^{b}, S(O)ₘN(R^{a}R^{b}), N(R^{a})S(O)ₘN(R^{a}R^{b}), OS(O)ₘN(R^{a}R^{b}), N(R^{a})S(O)ₘOR^{b}, C(O)R^{a}, OC(O)R^{a}, C(O)OR^{a}, OC(O)OR^{a}, N(R^{a})C(O)R^{b}, C(O)N(R^{a}R^{b}), N(R^{a})C(O)N(R^{a}R^{b}), OC(O)N(R^{a}R^{b}), N(R^{a})C(O)OR^{b}, C(NR^{a}R^{b})═N(R^{a}), N(R^{a})C(NR^{a}R^{b})═N(R^{a}), C(SR^{a})═N(R^{b}), C(OR^{a})═N(R^{b}), N(R^{a})C(SR^{a})═N(R^{b}) and heterocycle optionally substituted with oxo or R^{a};
or optionally when R² is C₅₋₇ cycloalkyl, C₆₋₁₄ aralkyl, C₅₋₇ cycloalkenyl, C₆₋₁₄ aryl or heterocycle R² may be fused to 5-7 membered carbocyclic or heterocyclic rings;
R³ is hydrogen, hydroxy, C₁₋₈ alkyl, C₁₋₈ haloalkyl, C₃₋₇ cycloalkyl, C₂₋₆ alkenyl, C₃₋₇ cycloalkenyl, C₃₋₆ alkynyl, N(R^{a}R^{b}), or heterocycle, each of which may be optionally substituted with one or more substituents independently selected from the group consisting of C₁₋₈ alkyl, C₁₋₈ haloalkyl, C₃₋₇ cycloalkyl, C₂₋₆ alkenyl, C₃₋₇ cycloalkenyl, C₃₋₆ alkynyl, halogen, oxo, CN, NO₂, OR^{a}, N(R^{a}R^{b}), S(O)ₘR^{a}, SR^{a}, OS(O)ₘR^{a}, S(O)ₘOR^{a}, OS(O)ₘOR^{a}, N(R^{a})S(O)ₘR^{b}, S(O)ₘN(R^{a}R^{b}), N(R^{a})S(O)ₘN(R^{a}R^{b}), OS(O)ₘN(R^{a}R^{b}), N(R^{a})S(O)ₘOR^{b}, C(O)R^{a}, OC(O)R^{a}, C(O)OR^{a}, OC(O)OR^{a}, N(R^{a})C(O)R^{b}, C(O)N(R^{a}R^{b}), N(R^{a})C(O)N(R^{a}R^{b}), OC(O)N(R^{a}R^{b}), N(R^{a})C(O)OR^{b}, C(NR^{a})═N(R^{b}), C(SR^{a})═N(R^{b}), C(OR^{a})═N(R^{b}), N(R^{a})C(NR^{a}R^{b})═N(R^{a}), N(R^{a})C(SR^{a})═N(R^{b}), N(R^{a})C(OR^{a})═N(R^{b}), and heterocycle optionally substituted by oxo or R^{a};
R^{a} and R^{b} are independently hydrogen, NO₂, OR^{c}, CN, N(R^{c}R^{d}), C(O)R^{c}, C(O)C(O)R^{c}, C(O)N(R^{c}R^{d}), C(O)C(O)N(R^{c}R^{d}), S(O)ₘR^{c}, SR^{c}, S(O)ₘN(R^{c}R^{d}), C₁₋₈ alkyl, C₁₋₈ haloalkyl, C₃₋₇ cycloalkyl, C₆₋₁₄ aralkyl, C₂₋₆ alkenyl, C₃₋₇ cycloalkenyl, C₃₋₆ alkynyl, C₆₋₁₄ aryl or heterocycle, each of which may be optionally substituted with one or more substituents independently selected from the group consisting of C₁₋₈ alkyl, C₁₋₈ haloalkyl, C₃₋₇ cycloalkyl, C₆₋₁₄ aralkyl, C₂₋₆ alkenyl, C₃₋₇ cycloalkenyl, C₃₋₆ alkynyl, C₆₋₁₄ aryl, CN, NO₂, OR^{c}, N(R^{c}R^{d}), S(O)ₘR^{c}, SR^{c}, OS(O)ₘR^{c}, S(O)ₘOR^{c}, OS(O)ₘOR^{c}, N(R^{c})S(O)ₘR^{d}, S(O)ₘN(R^{c}R^{d}), N(R^{c})S(O)ₘN(R^{c}R^{d}), OS(O)ₘN(R^{c}R^{d}), N(R^{c})S(O)ₘOR^{d}, C(O)R^{c}, OC(O)R^{c}, C(O)OR^{c}, OC(O)OR^{c}, N(R^{c})C(O)R^{d}, C(O)N(R^{c}R^{d}), N(R^{c})C(O)N(R^{c}R^{d}), OC(O)N(R^{c}R^{d}), N(R^{c})C(O)OR^{d}, C(NR^{c}R^{d})═N(R^{c}), C(SR^{c})═N(R^{d}), C(OR^{c})═N(R^{d}) and heterocycle;
Optionally, R^{a} and R^{b} may be linked together through one or more ring carbon atoms and/or ring heteroatoms including N, O, C(R^{c}R^{d}), C(O), S(O)ₘ, or S to form a saturated or unsaturated 3 to 8 membered carbocyclic or heterocyclic ring optionally substituted with oxo or R^{a};
R^{c} and R^{d} are independently hydrogen, C₁₋₈ alkyl, C₁₋₈ haloalkyl, C₃₋₇ cycloalkyl, C₆₋₁₄ aralkyl, C₂₋₆ alkenyl, C₃₋₇ cycloalkenyl, C₃₋₆ alkynyl, C₆₋₁₄ aryl or heterocycle;
Optionally, R^{c} and R^{d} may be linked together through one or more ring carbon atoms and/or ring heteroatoms including N, O, C(O) and S(O)ₘ, or S to form a saturated or unsaturated 3 to 8 membered carbocyclic or heterocyclic ring;
m is 1 or 2;
   or a pharmaceutically acceptable salt thereof.

The present invention also features a compound of formula (Ia) wherein:
R¹ is
(a) C₁₋₈alkoxy substituted with C₆₋₁₄aryl; OR^{a} or NR^{a}R^{b};
(b) C(O)NR^{a}R^{b};
(c) C₃₋₆alkynyl substituted with OR^{a} or NR^{a}R^{b};
(d) C₁₋₈alkylcarbonyl; or
(e) C₁₋₈alkyl substituted with OR^{a}; C(O)NR^{a}R^{b}; N(R^{a})S(O)ₘR^{b}; N(R^{a})C(O)R^{b}; NR^{a}R^{b}; or heterocycle optionally substituted with one or more oxo;
R² is C₁₋₈alkyl;
R3 is
(a) C₁₋₈alkyl optionally substituted with C₁₋₈alkyl, C₃₋₇cycloalkyl, OR^{a}, SR^{a}, C(O)N(R^{a}R^{b}), NR^{a}C(O)R^{b}, or heterocycle optionally substituted with oxo or R^{a};
(b) C₃₋₇cycloalkyl;
(c) C₁₋₈haloalkyl; or
(d) heterocycle optionally substituted with oxo;
R^{a} and R^{b} are independently hydrogen, NO₂, OR^{c}, CN, N(R^{c}R^{d}), C(O)R^{c}, C(O)C(O)R^{c}, C(O)N(R^{c}R^{d}), C(O)C(O)N(R^{c}R^{d}), S(O)ₘR^{c}, SR^{c}, S(O)ₘN(R^{c}R^{d}), C₁₋₈ alkyl, C₁₋₈ haloalkyl, C₃₋₇ cycloalkyl, C₆₋₁₄ aralkyl, C₂₋₆ alkenyl, C₃₋₇ cycloalkenyl, C₃₋₆ alkynyl, C₆₋₁₄ aryl or heterocycle, each of which may be optionally substituted with one or more substituents independently selected from the group consisting of C₁₋₈ alkyl, C₁₋₈ haloalkyl, C₃₋₇ cycloalkyl, C₆₋₁₄ aralkyl, C₂₋₆ alkenyl, C₃₋₇ cycloalkenyl, C₃₋₆ alkynyl, C₆₋₁₄ aryl, CN, NO₂, OR^{c}, N(R^{c}R^{d}), S(O)ₘR^{c}, SR^{c}, OS(O)ₘR^{c}, S(O)ₘOR^{c}, OS(O)ₘOR^{c}, N(R^{c})S(O)ₘR^{d}, S(O)ₘN(R^{c}R^{d}), N(R^{c})S(O)ₘN(R^{c}R^{d}), OS(O)ₘN(R^{c}R^{d}), N(R^{c})S(O)ₘOR^{d}, C(O)R^{c}, OC(O)R^{c}, C(O)OR^{c}, OC(O)OR^{c}, N(R^{c})C(O)R^{d}, C(O)N(R^{c}R^{d}), N(R^{c})C(O)N(R^{c}R^{d}), OC(O)N(R^{c}R^{d}), N(R^{c})C(O)OR^{d}, C(R^{c}R^{d})═N(R^{c}), C(SR^{c})═N(R^{d}), C(OR^{c})═N(R^{d}) and heterocycle;
Optionally, R^{a} and R^{b} may be linked together through one or more ring carbon atoms and/or ring heteroatoms including N, O, C(R^{c}R^{d}), C(O), S(O)ₘ, or S to form a saturated or unsaturated 3 to 8 membered carbocyclic or heterocyclic ring optionally substituted with oxo or R^{a};
R^{c} and R^{d} are independently hydrogen, C₁₋₈ alkyl, C₁₋₈ haloalkyl, C₃₋₇ cycloalkyl, C₆₋₁₄ aralkyl, C₂₋₆ alkenyl, C₃₋₇ cycloalkenyl, C₃₋₆ alkynyl, C₆₋₁₄ aryl or heterocycle;
Optionally, R^{c} and R^{d} may be linked together through one or more ring carbon atoms and/or ring heteroatoms including N, O, C(O) and S(O)ₘ, or S to form a saturated or unsaturated 3 to 8 membered carbocyclic or heterocyclic ring;
m is 1 or 2;
   or a pharmaceutically acceptable salt thereof.

The present invention also features a compound of formula (Ia) wherein:
R¹ is
(a) C₁₋₈alkoxy substituted with C₆₋₁₄aryl; OR^{a} (wherein R^{a} is C₁₋₈alkyl); or NR^{a}R^{b} (wherein R^{a} and R^{b} are independently hydrogen or C(O)R^{c} wherein R^{c} is C₁₋₈alkyl);
(b) C(O)NR^{a}R^{b} wherein R^{a} and R^{b} are independently hydrogen, C₁₋₈ alkyl, or C(O)R^{c} wherein R^{c} is C₁₋₈alkyl;
(c) C₃₋₆alkynyl substituted with OR^{a} (wherein R^{a} is C₁₋₈alkyl) or NR^{a} R^{b} (wherein R^{a} and R^{b} are independently hydrogen or C(O)R^{c} wherein R^{c} is C₁₋₈alkyl);
(d) C₁₋₈alkylcarbonyl; or
(e) C₁₋₈alkyl substituted with OR^{a} (wherein R^{a} is hydrogen or C(O)N(R^{c}R^{d}) wherein R^{c} and R^{d} are independently hydrogen or C₁₋₈alkyl); C(O)NR^{a}R^{b} (wherein R^{a} and R^{b} are hydrogen, C₁₋₈alkyl, CN or OR^{c} wherein R^{c} is hydrogen); N(R^{a})S(O)ₘR^{b} (wherein R^{a} and R^{b} are independently hydrogen or C₁₋₈alkyl and m is 2); N(R^{a})C(O)R^{b} (wherein R^{a} and R^{b} are C₁₋₈alkyl); NR^{a}R^{b} (wherein R^{a} and R^{b} are independently hydrogen, C₁₋₈alkyl or C(O)R^{c} wherein R^{c} is C₁₋₈alkyl); or heterocycle optionally substituted with one or more oxo;
R² is C₁₋₈alkyl;
R³ is C₁₋₈alkyl optionally substituted with OR^{a} wherein R^{a} is hydrogen or C₁₋₈alkyl;
   or a pharmaceutically acceptable salt thereof.

The present invention also features a compound of formula (Ia) wherein:
R¹ is
(a) C₁₋₈alkoxy substituted with C₆₋₁₄aryl; OR^{a} (wherein R^{a} is C₁₋₈alkyl); or NR^{a}R^{b} (wherein R^{a} and R^{b} are independently hydrogen or C(O)R^{c} wherein R^{c} is C₁₋₈alkyl);
R² is C₁₋₈alkyl;
R³ is C₁₋₈alkyl optionally substituted with OR^{a} (wherein R^{a} is hydrogen or C₁₋₈alkyl);
   or a pharmaceutically acceptable salt thereof.

The present invention features a compound selected from the group consisting of:
7-(2-Benzyloxy-4-fluoro-benzyl)-4-hydroxy-1-methyl-2-oxo-1,2-dihydro-[1,5]naphthyridine-3-carboxylic acid hydroxymethyl-amide;
7-(2-Benzyloxy-4-fluoro-benzyl)-4-hydroxy-1-methyl-2-oxo-1,2-dihydro-[1,5]naphthyridine-3-carboxylic acid methoxymethyl-amide;
7-(2-Carbamoylmethoxy-4-fluoro-benzyl)-4-hydroxy-1-methyl-2-oxo-1,2-dihydro-[1,5]naphthyridine-3-carboxylic acid(2-hydroxy-ethyl)-amide;
7-(2-Carbamoylmethoxy-4-fluoro-benzyl)-4-hydroxy-1-methyl-2-oxo-1,2-dihydro-[1,5]naphthyridine-3-carboxylic acid(2-methoxy-ethyl)-amide;
7-(4-Fluoro-2-methylcarbamoylmethoy-benzyl)-4-hydroxy-1-methyl-2-oxo-1,2-dihydro-[1,5]naphthyridine-3-carboxylic acid(2-hydroxy-ethyl)-amide;
7-(4-Fluoro-2-methylcarbamoylmethoxy-benzyl)-4-hydroxy-1-methyl-2-oxo-1,2-dihydro-[1,5]naphthyridine-3-carboxylic acid(2-methoxy-ethyl)-amide;
7-(2-Dimethylcarbamoylmethoxy-4-fluoro-benzyl)-4-hydroxy-1-methyl-2-oxo-1,2-dihydro-[1,5]naphthyridine-3-carboxylic acid(2-hydroxy-ethyl)-amide;
7-(2-Dimethylcarbamoylmethoxy-4-fluoro-benzyl)-4-hydroxy-1-methyl-2-oxo-1,2-dihydro-[1,5]naphthyridine-3-carboxylic acid(2-methoxy-ethyl)-amide;
7-[4-Fluoro-2-(2-morpholin-4-yl-ethoxy)-benzyl]-4-hydroxy-1-methyl-2-oxo-1,2-dihydro-[1,5]naphthyridine-3-carboxylic acid(2-hydroxy-ethyl)-amide;
7-[2-(2-Dimethylamino-ethoxy)-4-fluoro-benzyl]-4-hydroxy-1-methyl-2-oxo-1,2-dihydro-[1,5]naphthyridine-3-carboxylic acid(2-hydroxy-ethyl)-amide;
7-[4-Fluoro-2-(2-morpholin-4-yl-ethoxy)-benzyl]-4-hydroxy-1-methyl-2-oxo-1,2-dihydro-[1,5]naphthyridine-3-carboxylic acid(2-methoxy-ethyl)-amide;
7-[2-(2-Dimethylamino-ethoxy)-4-fluoro-benzyl]-4-hydroxy-1-methyl-2-oxo-1,2-dihydro-[1,5]naphthyridine-3-carboxylic acid(2-methoxy-ethyl)-amide;
7-[4-Fluoro-2-(3-methoxy-prop-1-ynyl)-benzyl]-4-hydroxy-1-methyl-2-oxo-1,2-dihydro-[1,5]naphthyridine-3-carboxylic acid(2-hydroxy-ethyl)-amide;
7-[2-(3-Acetylamino-prop-1-ynyl)-4-fluoro-benzyl]-4-hydroxy-1-methyl-2-oxo-1,2-dihydro-[1,5]naphthyridine-3-carboxylic acid(2-hydroxy-ethyl)-amide;
7-[4-Fluoro-2-(3-methoxy-prop-1-ynyl)-benzyl]-4-hydroxy-1-methyl-2-oxo-1,2-dihydro-[1,5]naphthyridine-3-carboxylic acid(2-methoxy-ethyl)-amide;
7-[2-(3-Acetylamino-prop-1-ynyl)-4-fluoro-benzyl]-4-hydroxy-1-methyl-2-oxo-1,2-dihydro-[1,5]naphthyridine-3-carboxylic acid(2-methoxy-ethyl)-amide;
7-(2-Acetyl-4-fluoro-benzyl)-4-hydroxy-1-methyl-2-oxo-1,2-dihydro-[1,5]naphthyridine-3-carboxylic acid(2-hydroxy-ethyl)-amide;
7-(2-Acetyl-4-fluoro-benzyl)-4-hydroxy-1-methyl-2-oxo-1,2-dihydro-[1,5]naphthyridine-3-carboxylic acid(2-methoxy-ethy)-amide;
7-(4-Fluoro-2-hydroxymethyl-benzyl)-4-hydroxy-1-methyl-2-oxo-1,2-dihydro-[1,5]naphthyridine-3-carboxylic acid(2-hydroxy-ethyl)-amide;
7-(4 -Fluoro-2-hydroxymethyl-benzyl)-4-hydroxy-1-methyl-2-oxo-1,2-dihydro-[1,5]naphthyridine-3-carboxylic acid(2-methoxy-ethyl)-amide;
Carbamic acid 5-fluoro-2-[8-hydroxy-7-(2-hydroxy-ethylcarbamoyl)-5-methyl-6-oxo-5,6-dihydro-[1,5]naphthyridin-3-ylmethyl]-benzyl ester;
Carbamic acid 5-fluoro-2-[8-hydroxy-7-(2-methoxy-ethylcarbamoyl)-5-methyl-6-oxo-5,6-dihydro-[1,5]naphthyridin-3-ylmethyl]-benzyl ester;
Ethyl-carbamic acid 5-fluoro-2-[8-hydroxy-7-(2-hydroxy-ethylcarbamoyl)-5-methyl-6-oxo-5,6-dihydro-[1,5]naphthyridin-3-ylmethyl]-benzyl ester;
Ethyl-carbamic acid 5-fluoro-2-[8-hydroxy-7-(2-methoxy-ethylcarbamoyl)-5-methyl-6-oxo-5,6-dihydro-[1,5]naphthyridin-3-ylmethyl]-benzyl ester;
7-(4-Fluoro-2-methoxymethyl-benzyl)-4-methoxy-1-methyl-2-oxo-1,2-dihydro-[1,5]naphthyridine-3-carboxylic acid(2-hydroxy-ethyl)-amide;
7-(2-Carbamoyl-4-fluoro-benzyl)-4-hydroxy-1-methyl-2-oxo-1,2-dihydro-[1,5]naphthyridine-3-carboxylic acid(2-methoxy-ethyl)-amide;
7-(4-Fluoro-2-piperidin-1-ylmethyl-benzyl)-4-hydroxy-1-methyl-2-oxo-1,2-dihydro-[1,5]naphthyridine-3-carboxylic acid hydroxymethyl-amide;
7-(4-Fluoro-2-piperidin-1-ylmethyl-benzyl)-4-hydroxy-1-methyl-2-oxo-1,2-dihydro-[1,5]naphthyridine-3 -carboxylic acid methoxymethyl-amide;
7-[4-Fluoro-2-(2-oxo-pyrrolidin-1-ylmethyl)-benzyl]-4-hydroxy-1-methyl-2-oxo-1,2-dihydro-[1,5]naphthyridine-3-carboxylic acid hydroxymethyl-amide;
7-[4-Fluoro-2-(2-oxo-pyrrolidin-1-ylmethyl)-benzyl]-4-hydroxy-1-methyl-2-oxo-1,2-dihydro-[1,5]naphthyridine-3-carboxylic acid methoxymethyl-amide;
7-(4-Fluoro-2-imidazol-1-ylmethyl-benzyl)-4-hydroxy-1-methyl-2-oxo-1,2-dihydro-[1,5]naphthyridine-3 -carboxylic acid hydroxymethyl-amide;
7-(4-Fluoro-2-imidazol-1-ylmethyl-benzyl)-4-hydroxy-1-methyl-2-oxo-1,2-dihydro-[1,5]naphthyridine-3-carboxylic acid methoxymethyl-amide;
7-[2-(1,1-Dioxo-1lambda*6*-[1,2]thiazinan-2-ylmethyl)-4-fluoro-benzyl]-4-hydroxy-1-methyl-2-oxo-1,2-dihydro-[1,5]naphthyridine-3-carboxylic acid(2-methoxy-ethyl)-amide;
7-[2-(Acetylamino-methyl)-4-fluoro-benzyl]-4-hydroxy-1-methyl-2-oxo-1,2-dihydro-[1,5]naphthyridine-3-carboxylic acid(2-hydroxy-ethyl)-amide;
7-[4-Fluoro-2-(2-methylcarbamoyl-ethyl)-benzyl]-4-hydroxy-1-methyl-2-oxo-1,2-dihydro-[1,5]naphthyridine-3-carboxylic acid(2-hydroxy-ethyl)-amide;
7-[4-Fluoro-2-(2-methylcarbamoyl-ethyl)-benzyl]-4-hydroxy-1-methyl-2-oxo-1,2-dihydro-[1,5]naphthyridine-3-carboxylic acid(2-methoxy-ethyl)-amide;
7-{2-[2-(Cyanomethyl-methyl-carbamoyl)-ethyl]-4-fluoro-benzyl}-4-hydroxy-1-methyl-2-oxo-1,2-dihydro-[1,5]naphthyridine-3-carboxylic acid(2-hydroxy-ethyl)-amide;
7-{2-[2-(Cyanomethyl-methyl-carbamoyl)-ethyl]-4-fluoro-benzyl}-4-hydroxy-1-methyl-2-oxo-1,2-dihydro-[1,5]naphthyridine-3-carboxylic acid(2-methoxy-ethyl)-amide;
7-[2-(2-Dimethylcarbamoyl-ethyl)-4-fluoro-benzyl]-4-hydroxy-1-methyl-2-oxo-1,2-dihydro-[1,5]naphthyridine-3-carboxylic acid(2-hydroxy-ethyl)-amide;
7-[2-(2-Dimethylcarbamoyl-ethyl)-4-fluoro-benzyl]-4-hydroxy-1-methyl-2-oxo-1,2-dihydro-[1,5]naphthyridine-3-carboxylic acid(2-methoxy-ethyl)-amide;
7-{4-Fluoro-2-[2-(2-hydroxy-1,1-dimethyl-ethylcarbamoyl)-ethyl]-benzyl}-4-hydroxy-1-methyl-2-oxo-1,2-dihydro-[1,5]naphthyridine-3-carboxylic acid(2-hydroxy-ethyl)-amide;
7-{4-Fluoro-2-[2-(2-hydroxy-1,1-dimethyl-ethylcarbamoyl)-ethyl]-benzyl}-4-hydroxy-1-methyl-2-oxo-1,2-dihydro-[1,5]naphthyridine-3-carboxylic acid(2-methoxy-ethyl)-amide;
7-(2-Carbamoyl-4-fluoro-benzyl)-4-hydroxy-1-methyl-2-oxo-1,2-dihydro-[1,5]naphthyridine-3-carboxylic acid(2-hydroxy-ethyl)-amide;
7-(2-Carbamoyl-4-fluoro-benzyl)-4-hydroxy-1-methyl-2-oxo-1,2-dihydro-[1,5]naphthyridine-3 -carboxylic acid(2-methoxy-ethyl)-amide;
7-(4-Fluoro-2-methylcarbamoyl-benzyl)-4-hydroxy-1-methyl-2-oxo-1,2-dihydro-[1,5]naphthyridine-3-carboxylic acid(2-hydroxy-ethyl)-amide;
7-(4-Fluoro-2-methylcarbamoyl-benzyl)-4-hydroxy-1-methyl-2-oxo-1,2-dihydro-[1,5]naphthyridine-3-carboxylic acid(2-methoxy-ethyl)-amide;
7-(2-Dimethylcarbamoyl-4-fluoro-benzyl)-4-hydroxy-1-methyl-2-oxo-1,2-dihydro-[1,5]naphthyridine-3-carboxylic acid(2-hydroxy-ethyl)-amide;
7-(2-Dimethylcarbamoyl-4-fluoro-benzyl)-4-hydroxy-1-methyl-2-oxo-1,2-dihydro-[1,5]naphthyridine-3-carboxylic acid(2-methoxy-ethyl)-amide;
7-[4-Fluoro-2-(4-methyl-piperazine-1-carbonyl)-benzyl]-4-hydroxy-1-methyl-2-oxo-1,2-dihydro-[1,5]naphthyridine-3-carboxylic acid(2-hydroxy-ethyl)-amide;
7-[4-Fluoro-2-(4-methyl-piperazine-1-carbonyl)-benzyl]-4-hydroxy-1-methyl-2-oxo-1,2-dihydro-[1,5]naphthyridine-3-carboxylic acid(2-methoxy-ethyl)-amide;
7-[4-Fluoro-2-(morpholine-4-carbonyl)-benzyl]-4-hydroxy-1-methyl-2-oxo-1,2-dihydro-[1,5]naphthyridine-3-carboxylic acid(2-hydroxy-ethyl)-amide;
7-[4-Fluoro-2-(morpholine-4-carbonyl)-benzyl]-4-hydroxy-1-methyl-2-oxo-1,2-dihydro-[1,5]naphthyridine-3-carboxylic acid(2-methoxy-ethyl)-amide;
7-(2-Ethylcarbamoyl-4-fluoro-benzyl)-4-hydroxy-1-methyl-2-oxo-1,2-dihydro-[1,5]naphthyridine-3-carboxylic acid(2-hydroxy-ethyl)-amide;
7-(2-Ethylcarbamoyl-4-fluoro-benzyl)-4-hydroxy-1-methyl-2-oxo-1,2-dihydro-[1,5]naphthyridine-3-carboxylic acid(2-methoxy-ethyl)-amide;
7-(2-Diethylcarbamoyl-4-fluoro-benzyl)-4-hydroxy-1-methyl-2-oxo-1,2-dihydro-[1,5]naphthyridine-3-carboxylic acid(2-hydroxy-ethyl)-amide;
7-(2-Diethylcarbamoyl-4-fluoro-benzyl)-4-hydroxy-1-methyl-2-oxo-1,2-dihydro-[1,5]naphthyridine-3-carboxylic acid(2-methoxy-ethyl)-amide;
7-(4-Fluoro-2-isopropylcarbamoyl-benzyl)-4-hydroxy-1-methyl-2-oxo-1,2-dihydro-[1,5]naphthyridine-3-carboxylic acid(2-hydroxy-ethyl)-amide;
7-(4-Fluoro-2-isopropylcarbamoyl-benzyl)-4-hydroxy-1-methyl-2-oxo-1,2-dihydro-[1,5]naphthyridine-3-carboxylic acid(2-methoxy-ethyl)-amide;
7-[2-(Ethyl-methyl-carbamoyl)-4-fluoro-benzyl]-4-hydroxy-1-methyl-2-oxo-1,2-dihydro-[1,5]naphthyridine-3-carboxylic acid(2-hydroxy-ethyl)-amide;
7-[2-(Ethyl-methyl-carbamoyl)-4-fluoro-benzyl]-4-hydroxy-1-methyl-2-oxo-1,2-dihydro-[1,5]naphthyridine-3-carboxylic acid(2-methoxy-ethyl)-amide;
7-{2-[Bis-(2-hydroxy-ethyl)-carbamoyl]-4-fluoro-benzyl}-4-hydroxy-1-methyl-2-oxo-1,2-dihydro-[1,5]naphthyridine-3-carboxylic acid(2-hydroxy-ethyl)-amide;
7-{2-[Bis-(2-hydroxy-ethyl)-carbamoyl]-4-fluoro-benzyl}-4-hydroxy-1-methyl-2-oxo-1,2-dihydro-[1,5]naphthyridine-3-carboxylic acid(2-methoxy-ethyl)-amide;
7-[4-Fluoro-2-(methanesulfonylamino-methyl)-benzyl]-4-hydroxy-1-methyl-2-oxo-1,2-dihydro-[1,5]naphthyridine-3-carboxylic acid(2-hydroxyethyl)-amide;
7-[4-Fluoro-2-(methanesulfonylamino-methyl)-benzyl]-4-hydroxy-1-methyl-2-oxo-1,2-dihydro-[1,5]naphthyridine-3-carboxylic acid(2-methoxyethyl)-amide;
7-(4-Fluoro-2-morpholin-4-ylmethyl-benzyl)-4-hydroxy-1-methyl-2-oxo-1,2-dihydro-[1,5]naphthyridine-3-carboxylic acid(2-hydroxy-ethyl)-amide;
7-(2-Dimethylaminomethyl-4-fluoro-benzyl)-4-hydroxy-1-methyl-2-oxo-1,2-dihydro-[1,5]naphthyridine-3-carboxylic acid(2-hydroxy-ethyl)-amide;
7-(4-Fluoro-2-morpholin-4-ylmethyl-benzyl)-4-hydroxy-1-methyl-2-oxo-1,2-dihydro-[1,5]naphthyridine-3-carboxylic acid(2-methoxy-ethyl)-amide;
7-(2-Dimethylaminomethyl-4-fluoro-benzyl)-4-hydroxy-1-methyl-2-oxo-1,2-dihydro-[1,5]naphthyridine-3-carboxylic acid(2-methoxy-ethyl)-amide;
7-{2-[(Acetyl-methyl-amino)-methyl]-4-fluoro-benzyl}-4-hydroxy-1-methyl-2-oxo-1,2-dihydro-[1,5]naphthyridine-3-carboxylic acid(2-hydroxy-ethyl)-amide;
7-{2-[(Acetyl-methyl-amino)-methyl]-4-fluoro-benzyl}-4-hydroxy-1-methyl-2-oxo-1,2-dihydro-[1,5]naphthyridine-3-carboxylic acid(2-methoxy-ethyl)-amide;
7-{4-Fluoro-2-[(methanesulfonyl-methyl-amino)-methyl]-benzyl}-4-hydroxy-1-methyl-2-oxo-1,2-dihydro-[1,5]naphthyridine-3-carboxylic acid(2-hydroxy-ethyl)-amide;
7-{4-Fluoro-2-[(methanesulfonyl-methyl-amino)-methyl]-benzyl}-4-hydroxy-1-methyl-2-oxo-1,2-dihydro-[1,5]naphthyridine-3-carboxylic acid(2-methoxy-ethyl)-amide;
7-(2-Dimethylcarbamoylmethyl-4-fluoro-benzyl)-4-hydroxy-1-methyl-2-oxo-1,2-dihydro-[1,5]naphthyridine-3-carboxylic acid(2-hydroxy-ethyl)-amide;
7-(2-Dimethylcarbamoylmethyl-4-fluoro-benzyl)-4-hydroxy-1-methyl-2-oxo-1,2-dihydro-[1,5]naphthyridine-3-carboxylic acid(2-methoxy-ethyl)-amide;
   and pharmaceutically acceptable salts thereof.

The present invention also features a compound selected from the group consisting of:
7-(2-Benzyloxy-4-fluoro-benzyl)-4-hydroxy-1-methyl-2-oxo-1,2-dihydro-[1,5]naphthyridine-3-carboxylic acid hydroxymethyl-amide;
7-(2-Benzyloxy-4-fluoro-benzyl)-4-hydroxy-1-methyl-2-oxo-1,2-dihydro-[1,5]naphthyridine-3-carboxylic acid methoxymethyl-amide;
7-[4-Fluoro-2-(2-morpholin-4-yl-ethoxy)-benzyl]-4-hydroxy-1-methyl-2-oxo-1,2-dihydro-[1,5]naphthyridine-3-carboxylic acid(2-hydroxy-ethyl)-amide;
7-[4-Fluoro-2-(3-methoxy-prop-1-ynyl)-benzyl]-4-hydroxy-1-methyl-2-oxo-1,2-dihydro-[1,5]naphthyridine-3-carboxylic acid(2-hydroxy-ethyl)-amide;
7-[4-Fluoro-2-(3-methoxy-prop-1-ynyl)-benzyl]-4-hydroxy-1-methyl-2-oxo-1,2-dihydro-[1,5]naphthyridine-3-carboxylic acid(2-methoxy-ethyl)-amide;
7-(2-Acetyl-4-fluoro-benzyl)-4-hydroxy-1-methyl-2-oxo-1,2-dihydro-[1,5]naphthyridine-3-carboxylic acid(2-hydroxy-ethyl)-amide;
Carbamic acid 5-fluoro-2-[8-hydroxy-7-(2-methoxy-ethylcarbamoyl)-5-methyl-6-oxo-5,6-dihydro-[1,5]naphthyridin-3-ylmethyl]-benzyl ester;
7-(4-Fluoro-2-methoxymethyl-benzyl)-4-hydroxy-1-methyl-2-oxo-1,2-dihydro-[1,5]naphthyridine-3-carboxylic acid(2-hydroxy-ethyl)-amide;
7-[2-(1,1-Dioxo-1lambda*6*-[1,2]thiazinan-2-ylmethyl)-4-fluoro-benzyl]-4-hydroxy-1-methyl-2-oxo-1,2-dihydro-[1,5]naphthyridine-3-carboxylic acid(2-hydroxy-ethyl)-amide;
7-{4-Fluoro-2-[(methanesulfonyl-methyl-amino)-methyl]-benzyl}-4-hydroxy-1-methyl-2-oxo-1,2-dihydro-[1,5]naphthyridine-3-carboxylic acid(2-hydroxy-ethyl)-amide;
   and pharmaceutically acceptable salts thereof

The present invention also features a compound selected from the group consisting of 7-(4-Fluoro-2-methoxymethyl-benzyl)-4-methoxy-1-methyl-2-oxo-1,2-dihydro-[1,5]naphthyridine-3-carboxylic acid(2-hydroxy-ethyl)-amide, 7-(4-Fluoro-2-methoxymethyl-benzyl)-4-hydroxy-1-methyl-2-oxo-1,2-dihydro-[1,5]naphthyridine-3-carboxylic acid(2-hydroxy-ethyl)-amide and pharmaceutically acceptable salts thereof.

Compounds of the present invention are useful as integrase inhibitors. One aspect of the instant invention relates to methods of treating or preventing viral infection, for example an HIV infection, in a biological sample comprising contacting the biological sample with compounds of formula (I) or (Ia) or pharmaceutically acceptable derivatives thereof. Another aspect of the instant invention relates to methods of treating or preventing viral infection, for example, an HIV infection, in a patient comprising administering to the patient a therapeutically effective amount of compounds of formula (I) or (Ia) or pharmaceutically acceptable derivatives thereof.

The compounds of the present invention are particularly suited to the treatment or prophylaxis of HIV infections and associated conditions. Reference herein to treatment extends to prophylaxis as well as the treatment of established infections, symptoms, and associated clinical conditions such as AIDS related complex (ARC), Kaposi's sarcoma, and AIDS dementia.

The compounds of the present invention exhibit advantages over previously disclosed integrase inhibitors, for example increased potency, metabolic stability, increased therapeutic index, or other pharmaceutical properties.

According to one embodiment of the invention, compounds of formula (I) or (Ia) or salts thereof may be formulated into compositions. In a preferred embodiment, the composition is a pharmaceutical composition, which comprises a compound of formula (I) or (Ia) and pharmaceutically acceptable carrier, adjuvant or vehicle. In one embodiment, the composition comprises an amount of a compound of the present invention effective to treat or prevent viral infection, for example an HIV infection, in a biological sample or in a patient. In another embodiment, compounds of this invention and pharmaceutical compositions thereof, which comprise an amount of a compound of the present innovation effective to inhibit viral replication or to treat or prevent a viral infection or disease or disorder, for example an HIV infection, and a pharmaceutically acceptable carrier, adjuvant or vehicle, may be formulated for administration to a patient, for example, for oral administration.

The present invention features compounds according to the invention for use in medical therapy, for example for the treatment or prophylaxis of a viral infection, for example an HIV infection and associated conditions. The compounds according to the invention are especially useful for the treatment of AIDS and related clinical conditions such as AIDS related complex (ARC), progressive generalized lymphadenopathy (PGL), Kaposi's sarcoma, thromobocytopenic purpura, AIDS-related neurological conditions such as AIDS dementia complex, multiple sclerosis or tropical paraperesis, anti-HIV antibody-positive and HIV-positive conditions, including such conditions in asymptomatic patients.

According to another aspect, the present invention provides a method for the treatment or prevention of the symptoms or effects of a viral infection in an infected patient, for example, a mammal including a human, which comprises administering to said patient a pharmaceutically effective amount of a compound according to the invention. According to one aspect of the invention, the viral infection is a retroviral infection, in particular an HIV infection.

The present invention further includes the use of a compound according to the invention in the manufacture of a medicament for administration to a subject for the treatment of a viral infection, in particular and HIV infection.

The compounds of the present invention may also be used in adjuvant therapy in the treatment of HIV infections or HIV-associated symptoms or effects, for example Kaposi's sarcoma.

The present invention further provides a method for the treatment of a clinical condition in a patient, for example, a mammal including a human which clinical condition includes those which have been discussed hereinbefore, which comprises treating said patient with a pharmaceutically effective amount of a compound according to the invention. The present invention also includes a method for the treatment or prophylaxis of any of the aforementioned diseases or conditions.

Reference herein to treatment extends to prophylaxis as well as the treatment of established conditions, disorders and infections, symptoms thereof, and associated. The above compounds according to the invention and their pharmaceutically acceptable derivatives may be employed in combination with other therapeutic agents for the treatment of the above infections or conditions. Combination therapies according to the present invention comprise the administration of a compound of the present invention or a pharmaceutically acceptable derivative thereof and another pharmaceutically active agent. The active ingredient(s) and pharmaceutically active agents may be administered simultaneously (i.e., concurrently) in either the same or different pharmaceutical compositions or sequentially in any order. The amounts of the active ingredient(s) and pharmaceutically active agent(s) and the relative timings of administration will be selected in order to achieve the desired combined therapeutic effect.

Examples of such therapeutic agents include, but are not limited to, agents that are effective for the treatment of viral infections or associated conditions. Among these agents are (1-alpha,2-beta,3-alpha)-9-[2,3-bis(hydroxymethyl)cyclobutyl]guanine [(−)BHCG, SQ-34514, lobucavir]; 9-[(2R,3R,4S)-3,4-bis(hydroxy methyl)2-oxetanosyl]adenine (oxetanocin-G); acyclic nucleosides, for example acyclovir, valaciclovir, famciclovir, ganciclovir, and penciclovir; acyclic nucleoside phosphonates, for example (S)-1-(3-hydroxy-2-phosphonyl-methoxypropyl)cytosine (HPMPC), [[[2-(6-amino-9H-purin-9-yl)ethoxy]methyl]phosphilnylidene]bis(oxymethylene)-2,2-dimethyl propanoic acid (bis-POM PMEA, adefovir dipivoxil), [[(1R)-2-(6-amino-9H-purin-9-yl)-1-methylethoxy]methyl]phosphonic acid (tenofovir), and (R)-[[2-(6-Amino-9H-purin-9-yl)-1-methylethoxy]methyl]phosphonic acid bis-(isopropoxycarbonyloxymethyl)ester (bis-POC-PMPA); ribonucleotide reductase inhibitors, for example 2-acetylpyridine 5-[(2-chloroanilino)thiocarbonyl)thiocarbonohydrazone and hydroxyurea; nucleoside reverse transcriptase inhibitors, for example 3′-azido-3′-deoxythymidine (AZT, zidovudine), 2′,3′-dideoxycytidine (ddC, zalcitabine), 2′,3′-dideoxyadenosine, 2′,3′-dideoxyinosine (ddI, didanosine), 2′,3′-didehydrothymidine (d4T, stavudine), (−)-beta-D-2,6-diaminopurine dioxolane (DAPD), 3′-azido-2′,3′-dideoxythymidine-5′-H-phosphophonate (phosphonovir), 2′-deoxy-5-iodo-uridine (idoxuridine), (−)-cis-1-(2-hydroxymethyl)-1,3-oxathiolane 5-yl)-cytosine (lamivudine), cis-1-(2-(hydroxymethyl)-1,3-oxathiolan-5-yl)-5-fluorocytosine (FTC), 3′-deoxy-3′-fluorothymidine, 5-chloro-2′,3′-dideoxy-3′-fluorouridine, (−)-cis-4-[2-amino-6-(cyclo-propylamino)-9H-purin-9-yl]-2-cyclopentene-1-methanol (abacavir), 9-[4-hydroxy-2-(hydroxymethyl)but-1-yl]-guanine (H2G), ABT-606 (2HM-H2G) and ribavirin; protease inhibitors, for example indinavir, ritonavir, nelfinavir, amprenavir, saquinavir, fosamprenavir, (R)-N-tert-butyl-3-[(2S,3S)-2-hydroxy-3-N-[(R)-2-N-(isoquinolin-5-yloxyacetyl)amino-3-methylthio-propanoyl]amino-4-phenylbutanoyl]-5,5-dimethyl-1,3-thiazolidine-4-carboxamide (KNI-272), 4R-(4alpha,5alpha,6beta)]-1,3-bis[(3-aminophenyl)methyl]hexahydro-5,6-dihydroxy-4,7-bis(phenylmethyl)-2H-1,3-diazepin-2-one dimethanesulfonate (mozenavir), 3-[1-[3-[2-(5-trifluoromethylpyridinyl)-sulfonylamino]phenyl]propyl]-4-hydroxy-6alpha-phenethyl-6beta-propyl-5,6-dihydro-2-pyranone (tipranavir), N′-[2(S)-Hydroxy-3(S)-[N-(methoxycarbonyl)-1-tert-leucylamino]-4-phenylbutyl-N^{alpha}-(methoxycarbonyl)-N′-[4-(2-pyridyl)benzyl]-L-tert-leucylhydrazide (BMS-232632), 3-(2(S)-Hydroxy-3(S)-(3-hydroxy-2-methylbenzamido)-4-phenylbutanoyl)-5,5-dimethyl-N-(2-methylbenzyl)thiazolidine-4(R)-carboxamide (AG-1776), N-(2(R)-hydroxy-1(S)-indanyl)-2(R)-phenyl-methyl-4(S)-hydroxy-5-(1-(1-(4-benzo[b]furanylmethyl)-2(S)-N′-(tert-butyl carboxamido)piperazinyl)pentanamide (MK-944A); interferons such as α-interferon; renal excretion inhibitors such as probenecid; nucleoside transport inhibitors such as dipyridamole, pentoxifylline, N-acetylcysteine (NAC), Procysteine, α-trichosanthin, phosphonoformic acid; as well as immunomodulators such as interleukin II or thymosin, granulocyte macrophage colony stimulating factors, erythropoetin, soluble CD₄ and genetically engineered derivatives thereof; non-nucleoside reverse transcriptase inhibitors (NNRTIs), for example nevirapine (BI-RG-587), alpha-((2-acetyl-5-methylphenyl)amino)-2,6-dichloro-benzeneacetamide (loviride), 1-[3-(isopropyl amino)-2-pyridyl]-4-[5-(methanesulfonamido)-1H-indol-2-ylcarbonyl]piperazine monomethanesulfonate (delavirdine), (10R,11S,12S)-12-Hydroxy-6,6,10,11-tetramethyl-4-propyl-11,12-dihydro-2H,6H,10H-benzo(1,2-b:3,4-b′:5,6-b″)tripyran-2-one ((+) calanolide A), (4S)-6-Chloro-4-[1E)-cyclopropyl ethenyl)-3,4-dihydro-4-(trifluoromethyl)-2(1H)-quinazolinone (DPC-083), (S)-6-chloro-4-(cyclopropyl ethynyl)-1,4-dihydro-4-(trifluoromethyl)-2H-3,1-benzoxazin-2-one (efavirenz, DMP 266), 1-(ethoxy methyl)-5-(1-methylethyl)-6-(phenylmethyl)-2,4(1H,3H)-pyrimidinedione (MKC-442), and 5-(3,5-dichloro phenyl)thio-4-isopropyl-1-(4-pyridyl)methyl-1H-imidazol-2-ylmethyl carbamate (capravirine); glycoprotein 120 antagonists, for example PRO-2000, PRO-542 and 1,4-bis[3-[(2,4-dichlorophenyl)carbonyl amino]-2-oxo-5,8-disodiumsulfanyl]naphthalyl-2,5-dimethoxyphenyl-1,4-dihydrazone (FP-21399); cytokine antagonists, for example reticulose (Product-R), 1,1′-azobis-formamide (ADA), 1,11-(1,4-phenylenebis (methylene))bis-1,4,8,11-tetraazacyclotetradecane octahydrochloride (AMD-3100); integrase inhibitors; and fusion inhibitors, for example T-20 and T-1249.

The present invention further includes the use of a compound according to the invention in the manufacture of a medicament for simultaneous or sequential administration with at least another therapeutic agent, such as those defined hereinbefore.

Compounds of the present invention may be administered with an agent known to inhibit or reduce the metabolism of compounds, for example ritonavir. Accordingly, the present invention features a method for the treatment or prophylaxis of a disease as hereinbefore described by administration of a compound of the present invention in combination with a metabolic inhibitor. Such combination may be administered simultaneously or sequentially.

In general a suitable dose for each of the above-mentioned conditions will be in the range of 0.01 to 250 mg per kilogram body weight of the recipient (e.g. a human) per day, preferably in the range of 0.1 to 100 mg per kilogram body weight per day and most preferably in the range 0.5 to 30 mg per kilogram body weight per day and particularly in the range 1.0 to 20 mg per kilogram body weight per day. Unless otherwise indicated, all weights of active ingredient are calculated as the parent compound of formula (I) or (Ia); for salts or esters thereof, the weights would be increased proportionally. The desired dose may be presented as one, two, three, four, five, six or more sub-doses administered at appropriate intervals throughout the day. In some cases the desired dose may be given on alternative days. These sub-doses may be administered in unit dosage forms, for example, containing 10 to 1000 mg or 50 to 500 mg, preferably 20 to 500 mg, and most preferably 50 to 400 mg of active ingredient per unit dosage form.

While it is possible for the active ingredient to be administered alone, it is preferable to present it as a pharmaceutical composition. The compositions of the present invention comprise at least one active ingredient, as defined above, together with one or more acceptable carriers thereof and optionally other therapeutic agents. Each carrier must be acceptable in the sense of being compatible with the other ingredients of the composition and not injurious to the patient.

Pharmaceutical compositions include those suitable for oral, rectal, nasal, topical (including transdermal, buccal and sublingual), vaginal or parenteral (including subcutaneous, intramuscular, intravenous, intradermal, and intravitreal) administration. The compositions may conveniently be presented in unit dosage form and may be prepared by any methods well known in the art of pharmacy. Such methods represent a further feature of the present invention and include the step of bringing into association the active ingredients with the carrier, which constitutes one or more accessory ingredients. In general, the compositions are prepared by uniformly and intimately bringing into association the active ingredients with liquid carriers or finely divided solid carriers or both, and then if necessary shaping the product.

The present invention further includes a pharmaceutical composition as hereinbefore defined wherein a compound of the present invention or a pharmaceutically acceptable derivative thereof and another therapeutic agent are presented separately from one another as a kit of parts.

Compositions suitable for transdermal administration may be presented as discrete patches adapted to remain in intimate contact with the epidermis of the recipient for a prolonged period of time. Such patches suitably contain the active compound 1) in an optionally buffered, aqueous solution or 2) dissolved and/or dispersed in an adhesive or 3) dispersed in a polymer. A suitable concentration of the active compound is about 1% to 25%, preferably about 3% to 15%. As one particular possibility, the active compound may be delivered from the patch by electrotransport or iontophoresis as generally described in Pharmaceutical Research 3(6), 318 (1986).

Pharmaceutical compositions of the present invention suitable for oral administration may be presented as discrete units such as capsules, caplets, cachets or tablets each containing a predetermined amount of the active ingredients; as a powder or granules; as a solution or a suspension in an aqueous or non-aqueous liquid; or as an oil-in-water liquid emulsion or a water-in-oil liquid emulsion. The active ingredient may also be presented as a bolus, electuary or paste.

A tablet may be made by compression or molding, optionally with one or more accessory ingredients. Compressed tablets may be prepared by compressing in a suitable machine the active ingredients in a free-flowing form such as a powder or granules, optionally mixed with a binder (e.g. povidone, gelatin, hydroxypropylmethyl cellulose), lubricant, inert diluent, preservative, disintegrant (e.g. sodium starch glycollate, cross-linked povidone, cross-linked sodium carboxymethyl cellulose) surface-active or dispersing agent. Molded tablets may be made by molding a mixture of the powdered compound moistened with an inert liquid diluent in a suitable machine. The tablets may optionally be coated or scored and may be formulated so as to provide slow or controlled release of the active ingredients therein using, for example, hydroxypropylmethyl cellulose in varying proportions to provide the desired release profile. Tablets may optionally be provided with an enteric coating, to provide release in parts of the gut other than the stomach.

Pharmaceutical compositions suitable for topical administration in the mouth include lozenges comprising the active ingredients in a flavored base, usually sucrose and acacia or tragacanth; pastilles comprising the active ingredient in an inert basis such as gelatin and glycerin, or sucrose and acacia; and mouthwashes comprising the active ingredient in a suitable liquid carrier.

Pharmaceutical compositions suitable for vaginal administration may be presented as pessaries, tampons, creams, gels, pastes, foams or spray. Pharmaceutical compositions may contain in addition to the active ingredient such carriers as are known in the art to be appropriate.

Pharmaceutical compositions for rectal administration may be presented as a suppository with a suitable carrier comprising, for example, cocoa butter or a salicylate or other materials commonly used in the art. The suppositories may be conveniently formed by admixture of the active combination with the softened or melted carrier(s) followed by chilling and shaping in molds.

Pharmaceutical compositions suitable for parenteral administration include aqueous and nonaqueous isotonic sterile injection solutions which may contain anti-oxidants, buffers, bacteriostats and solutes which render the pharmaceutical composition isotonic with the blood of the intended recipient; and aqueous and non-aqueous sterile suspensions which may include suspending agents and thickening agents; and liposomes or other microparticulate systems which are designed to target the compound to blood components or one or more organs. The pharmaceutical compositions may be presented in unit-dose or multi-dose sealed containers, for example, ampoules and vials, and may be stored in a freeze-dried (lyophilized) condition requiring only the addition of the sterile liquid carrier, for example water for injection, immediately prior to use. Extemporaneous injection solutions and suspensions may be prepared from sterile powders, granules and tablets of the kind previously described.

Unit dosage pharmaceutical compositions include those containing a daily dose or daily subdose of the active ingredients, as hereinbefore recited, or an appropriate fraction thereof.

It should be understood that in addition to the ingredients particularly mentioned above the pharmaceutical compositions of this invention may include other agents conventional in the art having regard to the type of pharmaceutical composition in question, for example, those suitable for oral administration may include such further agents as sweeteners, thickeners and flavoring agents.

The compounds of the present invention may be prepared according to the following reactions schemes and examples, or modifications thereof using readily available starting materials, reagents and conventional synthesis procedures. In these reactions, it is also possible to make use of variants which are known to those of ordinary skill in the art.

The compounds of the present invention may be readily prepared by methods outlined in Schemes 1-9 or the chemical sequences and schemes that are embedded within the examples or by methods known to one skilled in the art. It should be understood that the methods presented herein may be re-ordered or combined in different sequences or supplemented by literature methods known to one skilled in the art without undue experimentation to make compounds of the formula (I) or (Ia). These may be prepared by treating compounds such as 1c with amines (R³NH₂). These and other methods for the conversion of carboxylic esters and acid derivatives to amides are well known to those skilled in the art. For examples, see: March, J., Advanced Organic Chemistry, 4^{th} Edition; John Wiley & Sons, 1992, pp 419-424. Compounds such as 1c are prepared by treating 3-oxopropanoyl derivatives 1b with base (e.g. NaOMe or NaOEt) in protic solvents such as MeOH or EtOH. Oxopropanoyl derivatives 1b may be prepared by reacting amines 1a with malonylchloride derivatives in the presence of base. Alternatively, compounds 1b are prepared by heating a solution of amine 1a with a malonylchloride derivatives in a nonprotic solvent.

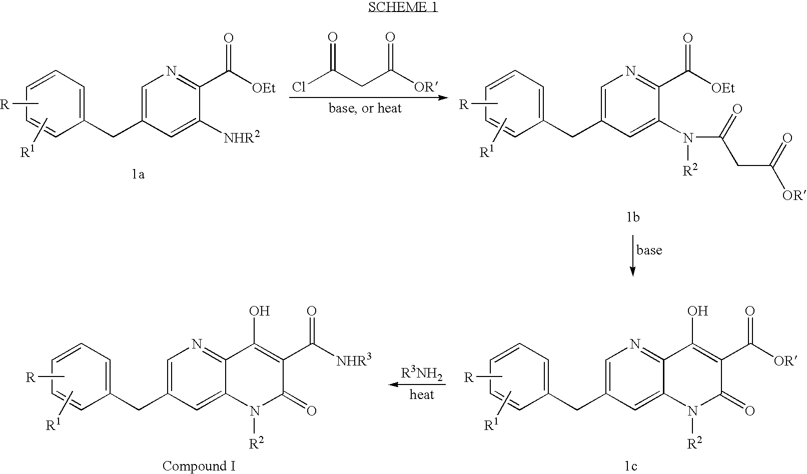

Amines 1a may be prepared by reductive amination of amines 2a with aldehydes and ketones as outlined in Scheme 2. For examples, of reductive amination reactions, see: March, J., Advanced Organic Chemistry, 4^{th} Edition; John Wiley & Sons, 1992, pp 898-900.

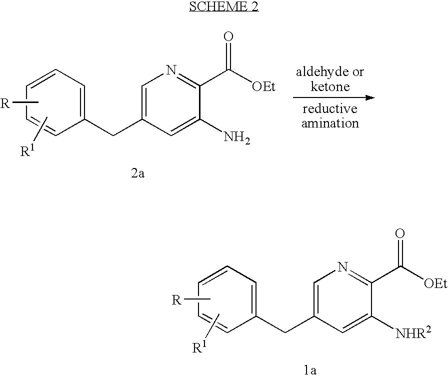

Amines 2a are readily prepared by methods outlined in Scheme 3. Heck reaction of aryl iodides 3a with allyl alcohol generates 3-arylproponals 3b. For examples of Heck reactions in the preparation of 3b, see: March, J., Advanced Organic Chemistry, 4^{th} Edition; John Wiley & Sons, 1992, pp 717-718. Treatment of 3b with formaldehyde in the presence of diethylamine hydrochloride affords requisite 2-benzylpropenals 3c. Reaction of 3c with diethyl 2-aminofumarate provides a pyridine diethyl ester 3d which may be hydrolyzed under basic conditions (e.g. NaOH) to the corresponding pyridine dicarboxylic acid 3e. For synthesis of diethyl 2-aminofumarate, see: Isobe, K.; Mohiri, C.; Sano, H.; Mohri, K.; Enomoto, H., Chem. Pharm. Bull., Vol. 37, 1989, pp 3236-3238. Treatment of 3e with acetic anhydride yields the corresponding cyclic anhydride 3f which is treated with EtOH at reflux to generate the pyridine carboxylic acid monoester 3g. Curtius rearrangement of 3g in the presence of t-BuOH yields the BOC-protected 3-aminopyridine derivative 3h which may be deprotected with TFA to afford the desired 3-aminopyridine compound 2a. For an example of a Curtius rearrangement of this type, see: Feiser, M., Reagents for Organic Synthesis, Vol. 11; John Wiley & Sons, 1984, p 222.

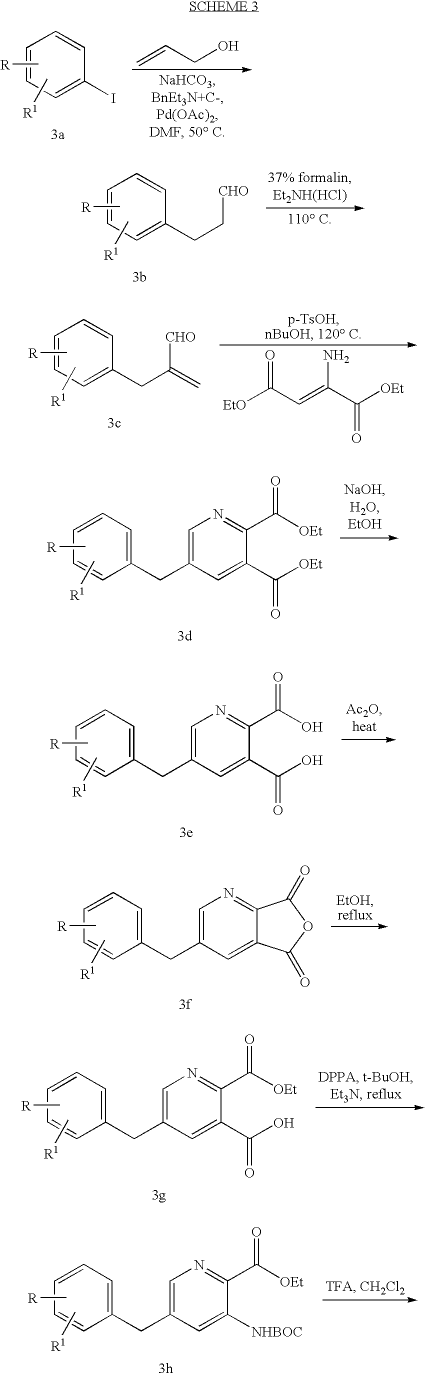

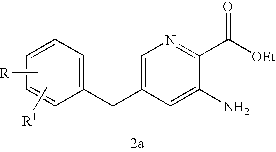

Another useful synthesis of a compound similar to la is shown in Scheme 4. Disubstituted pyridines such as 4a can be metallated in the presence of alkyllithium reagents or Grignard formation conditions by way of example. The reactive metallated species can then be exposed to an optionally substituted benzaldehyde (4b) at low temperature to form a diaryl carbinol such as 4c. The resultant benzylic alcohol can be converted to the corresponding diarylmethane derivative 4d by way of reduction. Typically conditions for reduction of an alcohol such as 4c involve catalytic hydrogenation or hydride reduction conditions. Catalytic hydrogenation conditions can typically involve the use of Pd/C in an alcoholic solvent or ethyl acetate as an example. A particularly useful reduction protocol well know to those skilled in the art for the reduction of benzylic alcohols involves treatment of 4c with triethylsilane in trifluoroacetic acid. Similarly, triethylsilane and a Lewis acid such as boron trifluoride etherate and the like can also be used in an inert solvent optionally with heating. The methyl ether in 4c is also able to be removed to produce the 2 hydroxypyridine moiety in the same pot as the reduction transformation. In cases where the methyl ether is not sufficiently cleaved, acidic conditions can be used to deblock the phenol. Typically these conditions include a strong acid such as HBr and the like optionally in a solvent such as acetic acid in some cases with heating. Pyridone 4d can be nitrated regioselectively to produce nitrophenol 4e. Typically conditions involve a acidic solvent such as TFA and a nitrating agent such as fuming nitric acid. This material can then be converted to a 2-bromo-pyridine derivative 4f by treatment with phosphorous oxybromide. In some cases the corresponding chloro derivative produced by use of phosphorous oxychloride may also be useful in the same reaction sequence. Typically conditions involve heating the pyridone 4e with phosphorous oxybromide or phosphorous oxychloride neat or in an inert solvent. Suitable solvents include but are not limited to toluene and 1,2-dichloroethane. In some cases a base may be added. Suitable bases may include diethylaniline by means of example. Palladium mediated carbonylation can be used to produce methyl ester 4g. Typically these conditions involve the use of a source of palladium (0) and an atmosphere of carbon monoxide optionally at ambient or increased pressures in the presence of a base. In many cases these reactions are best run at elevated temperatures. The catalyst can be tetrakistriphenylphosphine palladium (0) or palladium acetate and the like be way of example. Suitable bases such as triethylamine and the like are typically added. The nitro group in 4g can be reduced to form the aniline 4h using methods well known to those skilled in the art. Typical conditions involve catalytic hydrogenation. Suitable conditions may involve the use of palladium on carbon with an atmosphere of hydrogen at ambient or elevated pressures. In some cases the addition of iron metal can be particularly useful.

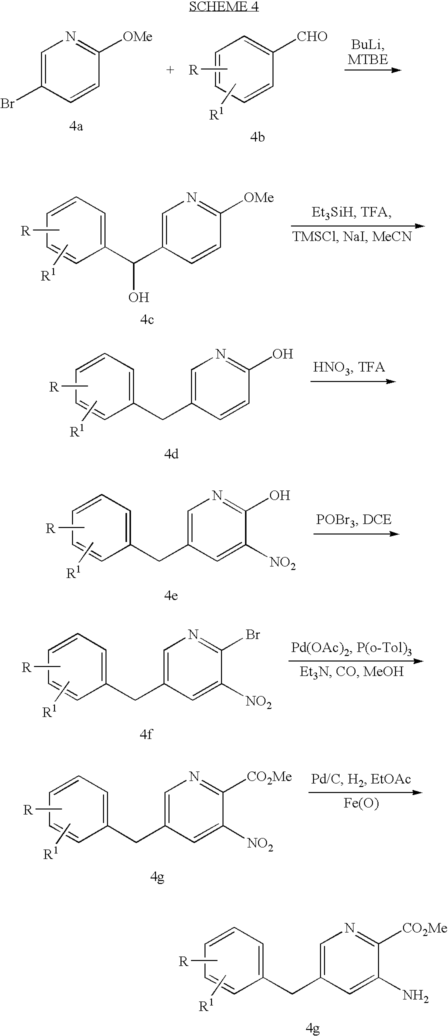

A different route to produce a compound similar to 1a is shown is Scheme 5. This strategy begins with a 3-fluoro-pyridine such as 5a. It is well precedented in the literature how to oxidize the pyridine 5a to form the corresponding pyridine N-oxide 5b (Sharpless, K. B. et. al. J. Org. Chem. 1998, 63, 1740). The literature method of Sakamoto et. al. (Chem. Pharm. Bull. 1985, 33, 565) can be used to form 2-cyano-3-fluoropyridine 5c by treatment of N-oxide 5b with TMSCN. This method is well known to regioselectively form the 2-nitrile. This material is able to be lithiated according to a modifications of methods described in the literature (WO 2004/019868) and treated with elemental iodine to form the 4-iodo derivative 5d. The 4-iodo derivative 5d can then be rearranged to the 5-iodo derivative 5e again according to a modification of the procedure outlined in the literature (WO 2004/019868). This 5-iodopyridine derivative can be subjected to a palladium mediated cross-coupling known to those skilled in the art as a Negishi-type coupling. Typically these cross-coupling reactions involve the reaction of an aryl halide with a alkyl zinc reagent. In this case reaction of iodide 5e with a benzyl zinc halide in the presence of a catalytic amount of a palladium (0) source resulted in formation of the 5-benzyl derivative 5g. The benzyl zinc halide can be prepared by literature methods or purchased from commercial sources. Typically, the catalyst is Pd(PPh₃)₄ and the like and the solvent is THF. The reaction optionally may be heated. An optionally substituted amine can be used to displace the 3-fluoro substituent in 5 g to produce 5 h. Typically this can be done by heating optionally in a microwave a mixture of the amine and 3-flouropyridine 5g in the amine neat or in an inert solvent to provide the 3-amino-2-cyano derivative 5h. The nitrile functionality may be hydrolyzed under acidic or basic conditions. A particularly useful method involves heating the nitrile in ethanolic sodium hydroxide to give the corresponding carboxylic acid 5i. The acid may then be converted to the corresponding ester using several methods well known in the literature. By way of example, particularly useful conditions involve the use of diazomethane, TMS-diazomethane and the like in a solvent such as ether or methanol/benzene respectively. Another particularly useful method for conversion of the acid to ester 5j involves the use of a base and alkylating agent. Typically, the alkylating agent is methyl iodide and the like and the base is potassium carbonate, triethylamine, sodium hydroxide and the like by way of example. This reaction can be performed optionally in an inert solvent such as DMF and the like.

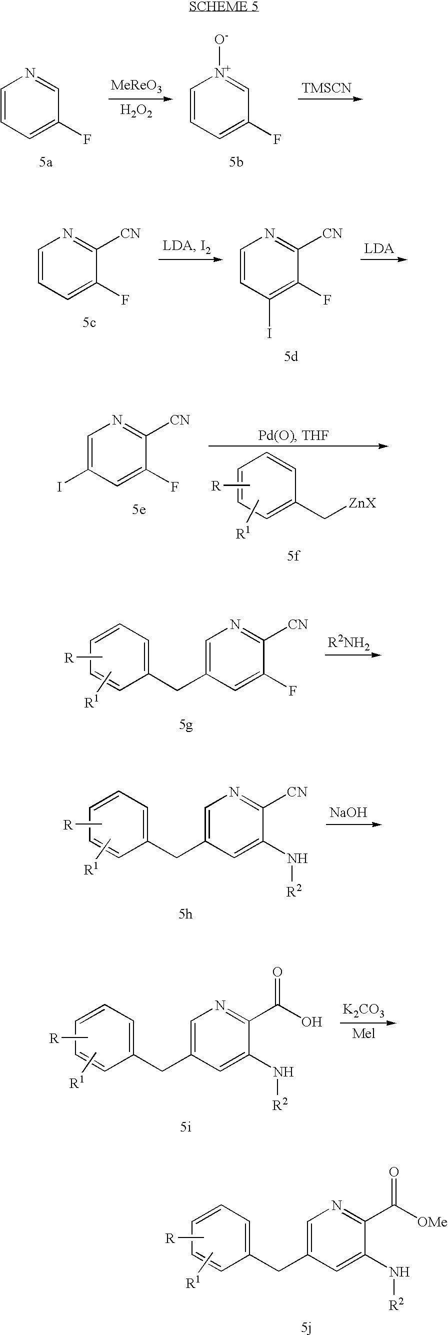

An analogous method to that shown in Scheme 5 can be used to form an intermediate 3,5-dibromo-2-cyanopyridine 6c (Scheme 6). A unique discovery with this system is the selective Negishi coupling to form intermediate 6e with a high level of selectivity. Dibromo derivative 6c can be treated with an optionally substituted benzyl zinc derivative 6d resulting in selective formation of the 5-benzyl product 6e. Typical conditions involved the use of Pd(PPh₃)₄ in an inert solvent such as THF and the like. The 3-bromosubstituent is particularly useful since it is well known that aryl bromides can be used for palladium mediated amination reactions known to those skilled in the art as Buchwald-Hartwig type couplings. This was particularly useful for the formation of compounds where R² was an optionally substituted aryl group however can be used in a general sense to form a wide variety of R² substituted compounds of the formula I. The remainder of the synthesis can proceed as shown in the previous Schemes.

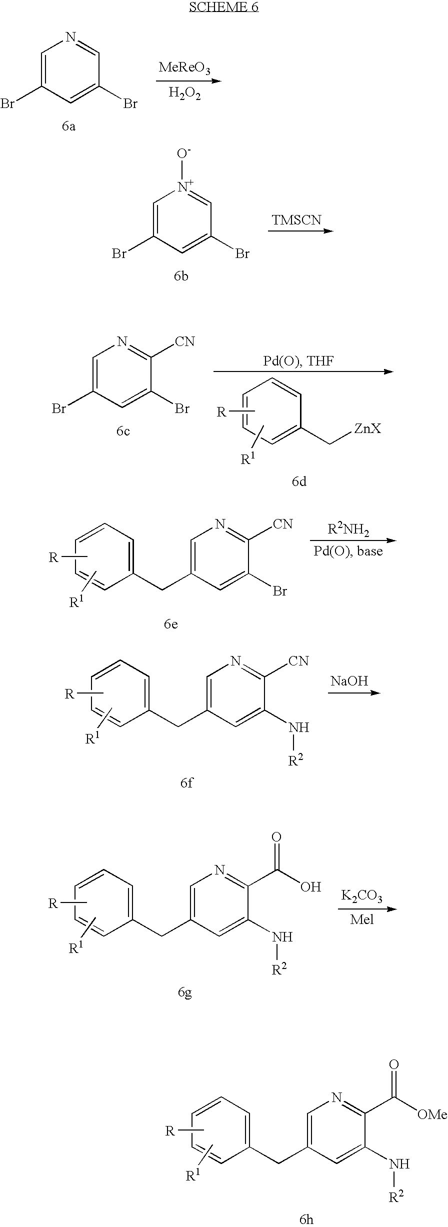

Another noteworthy method to convert a compound such as 2a to a selected group of compounds such as 7a where R² is aryl or heteroaryl involves the use of palladium mediated Buchwald-Hartwig reaction. Typically conditions for this type of reaction involve the use of a source of palladium (0) catalyst, a ligand and a base. By way of example conditions may use palladium acetate and the like as a catalyst. Suitable ligand may include but are not limited to phosphine ligands such as Xantphos. Bases include but are not limited to cesium carbonate and sodium tert-butoxide and the like.

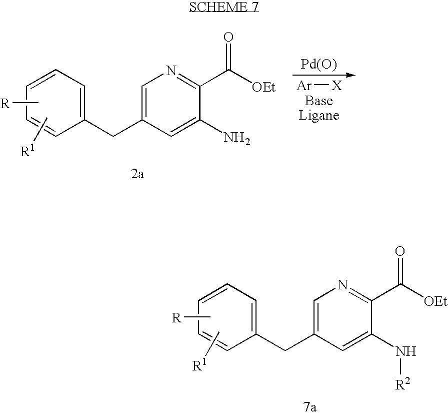

A useful method for conversion of a compound of formula 8a to one of the formula 1c involved the use of an alkylation (Scheme 8). Typically these type of reactions employ a base and an alkylating agent in an inert solvent. By way of example suitable bases include but are not limited to LDA, lithium hexamethyldisilazide, sodium hydride and the like. Alkylating agents include but are not limited to alkyl halides, triflates, mesylates, tosylates and the like.

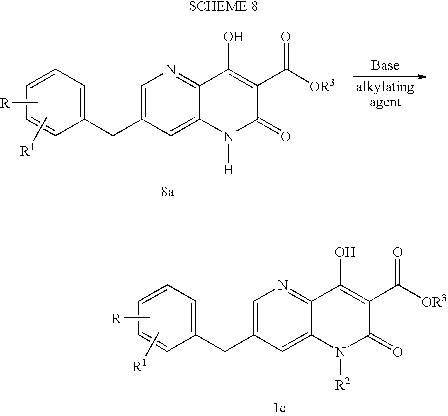

A useful method for conversion of a compound such as 2a to a higher substituted version such as 1a involves the method shown in Scheme 9. The 3 amino group can be activated for alkylation by conversion to a trifluoroacetamide or similar group such as shown in structure 9a. Typically this can be formed using trifluoroacetic anhydride or a similar reagent optionally with heating neat or in an inert solvent.

Trifluoroacetamide 9a can be alkylated using conditions known to those skilled in the art. Typical conditions may include the use of a base such as potassium carbonate and the like in an inert solvent such as acetonitrile or DMF. Alkylating agents include but are not limited to alkyl halides, triflates, mesylates and the like. Typically removal of the trifluoroacetamide can be accomplished by subjecting 9a to hydrolysis conditions. Suitable conditions typically include heating in an alcohol optionally in the presence of a base.

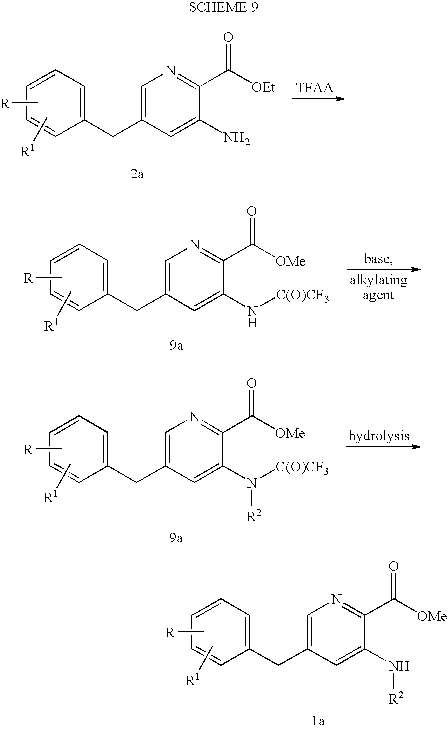

The following examples are intended for illustration only and are not intended to limit the scope of the invention in any way.

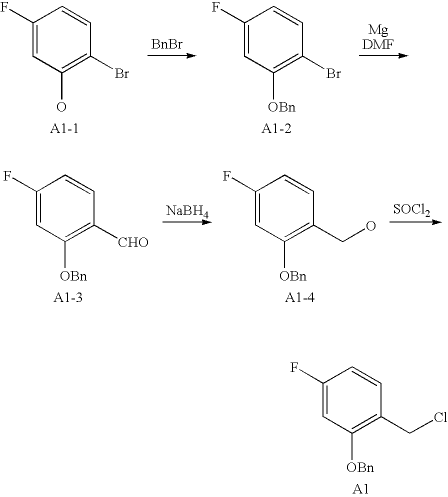

### Compound A1-2: 2-Benzyloxy-1-bromo-4-fluoro-benzene

To a solution of compound A1-1 (50.0 g, 131 mmol) in acetone (250 mL) were added potassium carbonate (37.8 g, 137 mmol) and benzyl bromide (31.0 mL, 131 mmol) at rt, and the mixture was refluxed at 80° C. for 2 h. Then, The mixture was treated with ice crushes and 1N HCl (300 mL), extracted with EtOAc. The extract was washed with water and brine. After dried over Na₂SO₄, the solvent was concentrated in vacuo to give 72.2 g of compound A1-2 (yield 98.0%) as pale yellow oil.

¹H NMR (CDCl₃) δ7.51-7.30(m, 6H), 6.68 (dd, J=10.5 Hz, 2.7 Hz, 1H), 6.62-6.50 (m, 1H), 5.13 (s, 2H).

### Compound A1-3: 2-Benzyloxy-4-fluoro-benzaldehyde

To a suspension of magnesium (6.36 g) in THF (40 mL) was added dropwise a solution of A1-2 (70.1 g, 249 mmol) in THF (240 mL) at 65° C. during 90 min period with stirring under N₂ atmosphere. After stirring at 65° C. for 30 min, the reaction mixture was treated with sat. NH₄Cl solution (500 mL). Then the mixture was extracted with EtOAc, the combined organic layers were washed with water, and dried over Na₂SO₄, the solvent was concentrated in vacuo to give 57.5 g of compound A1-3 (quant) as brown oil.

¹H NMR (CDCl₃) δ10.44 (s, 1H), 7.88 (t, 7.5 Hz, 1H), 7.42 (m, 5H), 6.74 (m, 2H), 5.17 (s, 2H).

### Compound A1-4: (2-Benzyloxy-4-fluoro-phenyl)-methanol

To a solution of compound A1-3 (57.1 g, 248 mmol) in EtOH (170 mL) was added NaBH₄ (4.69 g) at 0° C. and stirred for 1 h. The reaction mixture was treated with sat. NH₄Cl solution (300 mL), and then the mixture was extracted with EtOAc, the combined organic layers were washed with water and brine. After dried over Na₂SO₄, the solvent was concentrated in vacuo. The product mixture was purified by silicagel column chromatography (eluent: EtOAc/n-Hexane=1/5 to 1/3 v/v) to afford 43.4 g of compound A1-4 (yield=74%) as pale yellow oil.

¹H NMR (CDCl₃) δ7.41-7.33 (m, 5H), 7.25 (t, J=8.1 Hz, 1H), 6.70-6.62 (m, 2H), 5.08 (s, 2H), 4.68 (s, 2H), 2.10 (br s, 1H).

### Compound A1: 2-Benzyloxy-1-chloromethyl-4-fluoro-benzene

To a solution of compound A1-4 (42.4 g, 182 mmol) in CH₂Cl₂ (210 mL) was added dropwise thionyl chloride (15.6 mL, 219 mmol) at 0° C. during 7 min period, and then added DMF (1 drop). After stirring at rt for 30 min, the solvent was concentrated in vacuo. To the resulting residue were added cold-water (100 mL) and Et₂O (200 mL), and the mixture was neutralized to pH 7.2 with sat. NaHCO₃ solution. After neutralizing, the mixture was extracted with Et₂O several times and the combined organic layers were washed with water and brine, then dried over Na₂SO₄. The solvent was concentrated in vacuo to give a crude product. The crude product was purified by crystallization with n-Hexane to give 41.7 g of compound A-1 (yield=89.0%) as a pale yellow powder.

¹H NMR (CDCl₃) δ7.47-7.29 (m, 6H), 6.65 (m, 2H), 5.12 (s, 2H), 4.66 (s, 2H).

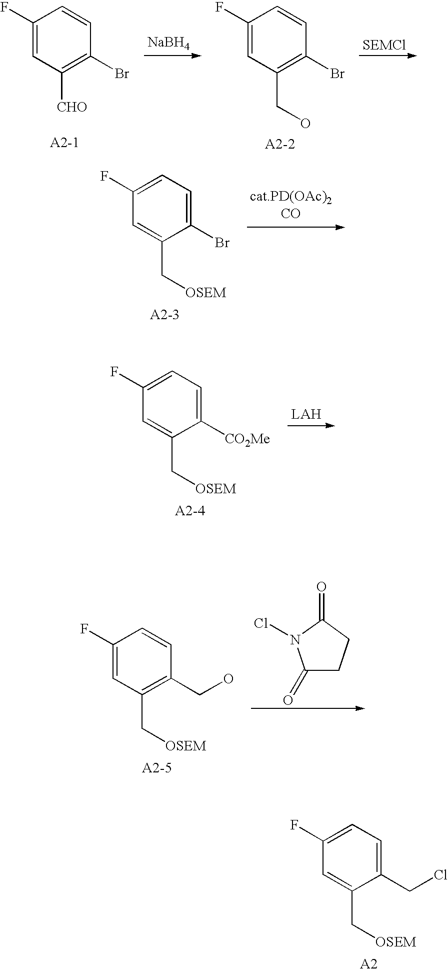

### Compound A2-2: (2-Bromo-5-fluoro-phenyl)-methanol

To a solution of compound A2-1 (75.0 g, 369 mmol) in EtOH (375 mL) was added a solution of NaBH₄ (4.47 g, 118 mmol) in water (9 mL) at 0° C., and the mixture was stirred at rt for 1 h. The mixture was treated with water and EtOAc, and extracted with EtOAc. The extract was washed with brine, dried over Na₂SO₄. The solvent was concentrated in vacuo to give 75.6 g of compound A2-2 (yield=99.8%) as a colorless solid.

¹H NMR (CDCl₃) δ 7.50-7.46(1H, m), 7.29-7.25(1H, m), 6.92-6.85(1H, m), 4.72(2H, s).

### Compound A2-3: [2-(2-Bromo-5-fluoro-benzyloxymethoxy)-ethyl]-trimethyl-silane

To a solution of compound A2-2 (75.6 g, 369 mmol) and diisopropylamine (57.7 g, 446 mmol) in CH₂Cl₂ (770 mL) was added SEMC1 (73.8 g, 443 mmol) under N₂ atmosphere at 0° C. After stirring at rt for 45 min, the mixture was treated with cold-water, and stirred for 10 min. The resulting mixture was extracted with n-Hexane, and washed with water and brine. After dried over Na₂SO₄, the solvent was concentrated in vacuo to give 131.0 g of compound A2-3 (quant).

¹H NMR (CDCl₃) δ 7.48-7.43(1H, m), 7.24-7.20(1H, m), 6.88-6.82(1H, m), 4.79(2H, s), 4.60(2H, s), 3.69-3.63(2H, m), 0.97-0.91(2H, m), 0.00(9H, s).

### Compound A2-4: 4-Fluoro-2-(2-trimethylsilanyl-ethoxymethoxymethyl)-benzoic acid methyl ester

To a solution of compound A2-3 (65.4 g, 184 mmol) in DMF (464 mL) and MeOH (186 mL) were added Et₃N (257 ml, 1844 mmol), Pd(OAc)₂ (8.28 g, 36.9 mmol) and diphenyl(phosphino)propane (19.0 g, 46.1 mmol) at rt. The mixture was stirred at 80° C. for 18 h under CO atmosphere. After the reaction mixture was treated with NH₄Cl solution and EtOAc, the insoluble materials were filtered off. The filtrate was washed with water, dried over Na₂SO₄. The solvent was concentrated in vacuo to afford the crude product. After adding acetone and n-Hexane, the resulting precipitates were filtered off. The filtrate was concentrated in vacuo to give 59.6 g of compound A2-4 (quant) as a red oil.

¹H NMR (CDCl₃) δ 8.01-7.97(1H, m), 7.44-7.40(1H, m), 7.01-6.95(1H, m), 5.00(2H, s), 4.81(2H, s), 3.86(3H, s), 3.70-3.63(2H, m), 0.97-0.91(2H, m), 0.00(9H, s).

### Compound A2-5: [4-Fluoro-2-(2-trimethylsitanyl-ethoxymethoxymethyl)-phenyl]-methanol

To a suspension of LiAlH₄ (14.0 g, 369 mmol) in Et₂O (1000 mL) was added dropwise a solution of compound A2-4 (119 g, 184 mmol) in Et₂O (200 mL) during 70 min period at 0° C., and stirred for 75 min. After water (14 mL) and 2N NaOH solution (14 mL) were added to the reaction mixture, Et₂O layer was separated. The Et₂O layer was dried over Na₂SO₄, the solvent was concentrated in vacuo to give 100 g of compound A2-5 (yield=95%) as a red oil.

¹H NMR (CDCl₃) δ 7.35-7.31(1H, m), 7.10-7.06(1H, m), 7.00-6.94(1H, m), 4.72(2H, s), 4.65(2H, s), 4.61(2H, s), 3.64-3.58(2H, m), 0.94-0.88(2H, m), 0.00(9H, s).

### Compound A2: [2-(2-Chloromethyl-5-fluoro-benzyloxymethoxy)-ethyl]-trimethyl-silane

To a solution of compound A2-5 (100 g, 350 mmol) in THF (1000 mL) was added P(Ph)₃ (96.5 g, 368 mmol) with stirring. NCS (49.1 g, 368 mmol) was added to the solution, and the mixture was stirred at rt for 1 h. The resulting precipitates were filtered off, and the filtrate was concentrated in vacuo. The residual oil was purified by distillation (148-154° C./2 mmHg) to give 93.2 g of compound A2 (yield=87.0%) as a colorless oil.

¹H NMR (CDCl₃) δ 7.34-7.29(1H, m), 7.16-7.12(1H, m), 6.98-6.92(1H, m), 4.75(2H, s), 4.70(2H, s), 4.62(2H, s), 3.67-3.62(2H, m), 0.96-0.91(2H, m), 0.00(9H, s).

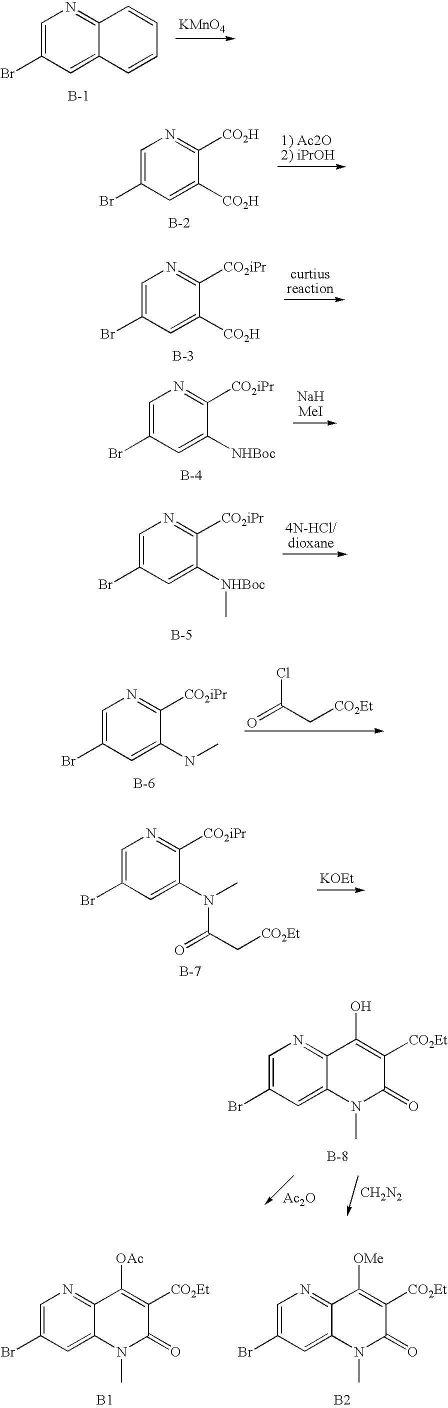

### Compound B-2: 5-Bromo-pyridine-2,3-dicarboxylic acid

To a mixture of 3-bromoquinoline (10 ml, 72.7 mmol) and water (200 mL) was added KMnO₄ (69.0 g, 436 mmol) at 6 portion each 15 min at 80° C. with stirring. After allowing the reaction to cool to rt, MeOH (20 mL) was added to the solution. The resulting mixture was washed with toluene (100 mL), and the aqueous layer was adjusted to pH 1 with conc. HCl. The mixture was extracted twice with EtOAc/THF (100 mL/50 mL). The combined extracts were washed with brine, dried over Na₂SO₄, and the solvent was removed in vacuo. Water was added to the residue, the resulting insoluble materials were filtered off. Isobutyl acetate was added to the filtrate, and then water was removed in vacuo. The resulting precipitates were collected by filtration, and dried to give 4.45 g of compound B-2 (yield=25%) as a colorless crystal.

¹H NMR (DMSO-d6) δ 12.50(1H, s), 8.90(1H, d, J=2.1 Hz), 8.43(1H, d, J=2.1 Hz).

### Compound B-3: 5-Bromo-pyridine-2,3-dicarboxylic acid 2-isopropyl ester

A mixture of compound B-2 (179 g, 728 mmol) and acetic anhydride (1250 mL) was heated at 120° C. for 1.5 h. After allowing the reaction to cool, acetic anhydride was removed in vacuo. After 2-propanol was added to the residue, the resulting mixture was heated at reflux for 13 h. After allowing the reaction to cool, the solvent was removed in vacuo. The crude product was purified by crystallization with acetone/(ⁱPr)₂O to give 77.8 g of compound B-4 (yield=37.0%) as a brown crystal.

¹H NMR (CDCl₃) δ 8.88(1H, d, J=2.1 Hz), 8.44(1H, d, J=2.1 Hz), 7.70(1H, bs), 5.41-5.28(1H, m), 1.40(6H, d, J=6.3 Hz).

### Compound B-4: 5-Bromo-3-tert-butoxycarbonylamino-pyridine-2-carboxylic acid isopropyl ester

To a solution of compound B-3 (101 g, 352 mmol) in ^{t}-BuOH (1014 mL) were added Et₃N (147 ml, 1055 mmol) and diphenyl(phosphoryl)azide (94.6 ml, 422 mmol) at rt under N₂ atmosphere with stirring. After the mixture was heated at reflux for 2.5 h, allowed cooling to rt. The reaction mixture was extracted with EtOAc, washed with sat.NaHCO₃ solution and brine. After the solvent was concentrated in vacuo, a mixture of acetone/n-Hexane was added to the residue. The resulting insoluble materials were removed, and then the solvent was concentrated in vacuo. The resulting product was purified by crystallization with (ⁱPr)₂O/n-Hexane to give 104 g of compound B-4 (yield=82.0%) as a off-brown crystal

¹H NMR (CDCl₃) δ 10.31(1H, s), 9.12(1H, d, J=2.0 Hz), 8.39(1H, d, J=2.0 Hz), 5.36-5.29(1H, m), 1.54(9H, s), 1.45(6H, d, J=6.3 Hz).

### Compound B-5: 5-Bromo-3-(tert-butoxycarbonyl-methyl-amino)-pyridine-2-carboxylic acid isopropyl ester

NaH (60%, 13.9 g, 347 mmol) was added at 7 portions to a solution of compound B-4 (104 g, 289 mmol) in THF/DMF (416 mL/623 mL) at 9° C. under N₂ atmosphere. After MeI (27 ml, 434 mmol) was added dropwise to the mixture, the reaction mixture was stirred at rt for 75 min. The resulting mixture was treated with NH₄Cl solution, extracted twice with EtOAc. The extract was washed with water and brine, dried over Na₂SO₄, and then the solvent was concentrated in vacuo to give 116 g of compound B-5 (quant) as a red oil.

¹H NMR (CDCl₃) δ 8.64(1H, s), 7.76(1H, s), 5.29-5.23(1H, m), 3.23(3H, s), 1.39(6H, d, J=6.3 Hz), 1.34(9H, s).

### Compound B-6: 5-Bromo-3-methylamino-pyridine-2-carboxylic acid isopropyl ester

A solution of compound B-5 (116 g, 289 mmol) in 4N HCl (1,4 dioxane solution) was stirred at rt for 1.5 h. After removal of the solvent, ice crushes and sat. NaHCO₃ solution were added to the residue. The resulting mixture was extracted twice with EtOAc, washed with brine, dried over Na₂SO₄. The solvent was concentrated in vacuo, and then the resulting product was purified by crystallization with n-Hexane to give 71.9 g of compound B-6 (yield=91.0%) as a yellow crystal.

¹H NMR (CDCl₃) δ 8.03(1H, d, J=1.8 Hz), 7.80(1H, brs), 7.20(1H, d, J=1.8 Hz), 5.32-5.24(1H, m), 2.90(3H, d, J=5.0 Hz), 1.43(6H, d, J=6.4 Hz).

### Compound B-7: 5-Bromo-3-[(2-ethoxycarbonyl-acetyl)-methyl-amino]-pyridine-2-carboxylic acid isopropyl ester

To a suspension of compound B-6 (2.33 g, 8.53 mmol) in THF (14 mL) was added ethyl-3-chloro-3-oxo-propionate (1.20 ml, 9.37 mmol). The mixture was heated at 120° C. in a sealed tube in a microwave for 5 min. After cooling, sat. NaHCO₃ solution was added to the mixture, and the mixture was extracted twice with EtOAc. The extract was washed with brine, dried over Na₂SO₄, and then the solvent was concentrated in vacuo. The residue was purified by crystallization with n-Hexane to give 2.81 g of compound B-7 (yield=85%) as a pale brown crystal.

¹H NMR (CDCl₃) δ 8.80(1H, d, J=1.8 Hz), 7.93(1H, d, J=1.8 Hz), 5.35-5.27(1H, m), 4.16-4.10(2H, m), 3.24(3H, s), 3.15(2H, d, J=10.8 Hz), 1.37(6H, d, J=6.4 Hz), 1.24(3H, J=7.2 Hz).

### Compound B-8: 7-Bromo-4-hydroxy-1-methyl-2-oxo-1,2-dihydro-[1,5]naphthyridine-3-carboxylic acid ethyl ester

To a solution of compound B-7 (88.0 g, 227 mmol) in DMF (880 mL) was added dropwise KOEt in EtOH (178 ml, 24 wt %, 454 mmol) at 0° C. under N₂ atmosphere, and the mixture was stirred at rt for 1 h. The reaction mixture was adjusted to pH 4 with 2N HCl at 0° C., and extracted several times with EtOAc. The combined extracts were dried over Na₂SO₄, and then concentrated in vacuo. The resulting product was triturated with Et₂O to give 68.3 g of compound B-8 (yield=92.0%) as a pale brown crystal.

¹H NMR (CDCl₃) δ 13.98(1H, bs), 8.66(1H, d, J=1.8 Hz), 7.85(1H, d, J=1.8 Hz), 4.53(2H, q, J=7.2 Hz), 3.63(3H, s), 1.49(3H, t, J=7.2 Hz).

### Compound B1: 4-Acetoxy-7-bromo-1-methyl-2-oxo-1,2-dihydro-[1,5]naphthyridine-3-carboxylic acid ethyl ester

A mixture of compound B-8 (26.1 g) and acetic anhydride (200 mL) was heated at 130° C. for 1.5 hr with stirring. After removal of the solvent, furthermore residual solvent was evaporated twice with toluene at reduced pressure. The resulting product was triturated with Et₂O to give 27.4 g of compound B1 (yield=93.0%) as a pale brown crystal.

¹H NMR (CDCl₃) δ 8.58(1H, d, J=1.8 Hz), 7.87(1H, d, J=1.8 Hz), 4.44(2H, q, J=7.2 Hz), 3.68(3H, s), 2.43(3H, s), 1.39(3H, t, J=7.2 Hz).

### Compound B2: 7-Bromo-4-methoxy-1-methyl-2-oxo-1,2-dihydro-[1,5]naphthyridine-3-carboxylic acid ethyl ester

Diazomethane solution in Et₂O was prepared from N-methyl-nitroso-urea (22.1 g) and a solution of KOH (17.8 g) in H₂O (12.5 mL). To a suspension of compound B-8 (2.0 g, 6.23 mmol) in Et₂O (50 mL) was added above-mentioned diazometan solution at 0° C. The reaction mixture was stirred at rt for 30 min, and then quenched with acetic acid. The resulting mixture was extracted with EtOAc, and the organic layer was washed successively with sat.NaHCO₃ solution, water and brine. After dried over Na₂SO₄, the solvent was concentrated in vacuo. The resulting product was purified by silicagel column chromatography (eluent: n-Hexane/EtOAc/MeOH=25/25/1 v/v/v) to give 1.20 g of compound B2 (yield=56.0%) as a colorless crystal.

¹H NMR (CDCl₃) δ 8.62(1H, d, J=1.8 Hz), 7.85(1H, d, J=1.8 Hz), 4.46(2H, q, J=7.2 Hz), 4.24(3H, s), 3.64(3H, s), 1.42(3H, t, J=7.2 Hz).

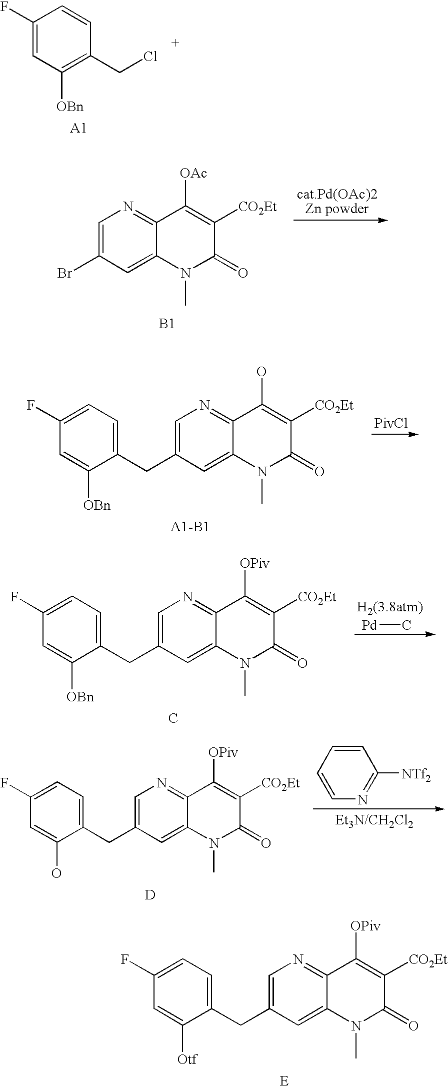

### Compound A1-B1: 7-(2-Benzyloxy-4-fluoro-benzyl)-4-hydroxy-1-methyl-2-oxo-1,2-dihydro-[1,5]naphthyridine-3-carboxylic acid ethyl ester

To a suspension of Zn (8.21 g, 125.5 mmol) in THF (200 mL) was added dropwise compound A1 (26.2 g, 104.5 mmol) at rt, and then added 1,2-dibromoethane (0.50 ml) and trimethylsilyl chloride (0.50 ml). After the reaction mixture was heated at 60° C. for 90 min, the mixture was cooled to rt. To the reaction mixture were added P(Ph)₃ (1.37 g, 5.23 mmol), Pd(OAc)₂ (587 mg, 2.62 mmol) and compound B1 (19.3 g, 52.3 mmol), and heated at 60° C. for 30 min with stirring. After the reaction mixture was cooled to rt, the mixture was treated with 2N HCl (20 mL), extracted with EtOAc. The extract was washed with water and brine, and dried over Na₂SO₄. The solvent was concentrated in vacuo to afford 30.5 g of crude compound A1-B1.

### Compound C: 7-(2-Benzyloxy-4-fluoro-benzyl)-4-(2,2-dimethyl-propionyloxy)-1-methyl-2-oxo-1,2-dihydro-[1,5]naphthyridine-3-carboxylic acid ethyl ester

To a solution of compound A1-B1 in pyridine (103 mL) was added pivaloyl chloride (5.5 ml, 44.6 mmol) at 0° C. and stirred at rt for 1 h. The resulting mixture was treated with water, and extracted twice with EtOAc and dried over Na₂SO₄. The solvent was concentrated in vacuo to afford the crude product, the product mixture was purified by silicagel column chromatography (eluent: n-Hexane/EtOAc=3/2 v/v) to give 9.32 g of compound C (yield=77.0% from compound B1).

¹H NMR (CDCl₃) δ 8.34(1H, d, J=1.4 Hz), 7.36-7.33(4H, m), 7.26-7.17(3H, m), 6.70-6.64(2H, m), 4.94(2H, s), 4.39(2H, q, J=7.2 Hz), 4.03(2H, s), 3.31(3H, s), 1.41(9H, s), 1.37(3H, t, J=7.2 Hz).

### Compound D: 4-(2,2-Dimethyl-propionyloxy)-7-(4-fluoro-2-hydroxy-benzyl)-1-methyl-2-oxo-1,2-dihydro-[1,5]naphthyridine-3-carboxylic acid ethyl ester

To a solution of compound C (10.9 g, 19.9 mmol) in 1,4-dioxane was added 10% Pd-C (4.36 g) at rt, and stirred for 18 h at 3.5 atm under H₂ atmosphere. After filtration through Celite, the filtrate was concentrated in vacuo. The residue was washed with EtOAc to give 8.99 g of compound D (yield=99.0%) as a colorless crystal.

¹H NMR (CDCl₃) δ 9.25(1H, bs), 8.44(1H, d, J=1.7 Hz), 7.54(1H, d, J=1.7 Hz), 7.07-7.02(1H, m), 6.66-6.62 (1H, m), 6.55-6.50(1H, m), 4.38(2H, q, J=7.2 Hz), 4.03(2H, s), 3.63(3H, s), 1.41(9H, s), 1.36(3H, t, J=7.2 Hz).

### Compound E: 4-(2,2-Dimethyl-propionyloxy)-7-(4-fluoro-2-trifluoromethanesulfonyloxy-benzyl)-1-methyl-2-oxo-1,2-dihydro-[1,5]naphthyridine-3-carboxylic acid ethyl ester

To a mixture of compound D (5.0 g, 10.95 mmol) and Bis(trifluoromethylsulfonyl)amino]pyridine (7.45 g, 20.81 mmol) in CH₂Cl₂ (170 ml) was added Et₃N (2.29 ml, 16.43 mmol) under N₂ atmosphere at 0° C., and was stirred for 5 min. After the reaction mixture was concentrated in vacuo, the residue was poured into ice/water. The mixture was extracted with EtOAc (300 mL) and washed with brine, then dried over Na₂SO₄. The solvent was concentrated to afford the crude purplish red solid (10 g), the solid was dissolved in CH₂Cl₂ (40 ml) and purified by silicagel column chromatography (eluent: n-Hexane/EtOAc=2/1 to 1/1 v/v) to afford 5.1 g of the product. Additionally, the solid product was washed with cold (ⁱPr)₂O to give 4.6 g of compound E (yield=72.1%) as a crystal.

¹H NMR (CDCl₃) δ 8.36(1H, d, J=1.8 Hz), 7.51(1H, d, J=1.8 Hz), 7.30-7.20(1H, m), 7.15-7.05(2H, m), 4.40(2H, quart, J=7.2 Hz), 4.18(2H, s), 3.65(3H, s), 1.41(9H, s), 1.38(3H, trip, J=7.2 Hz).

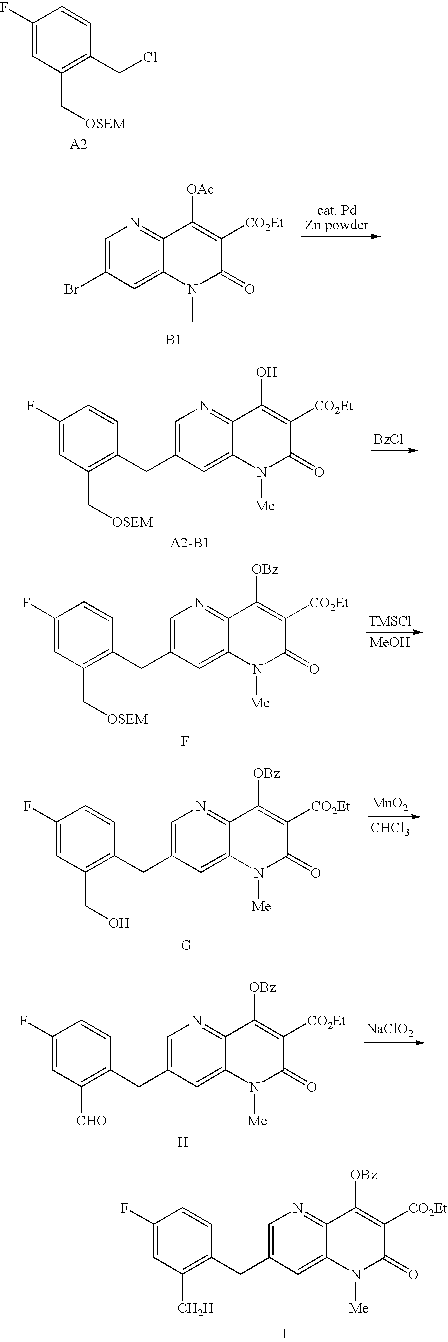

### Compound A2-B1: 7-[4-Fluoro-2-(2-trimethylsilanyl-ethoxymethoxymethyl)-benzyl]-4-hydroxy-1-methyl-2-oxo-1,2-dihydro-[1,5]naphthyridine-3-carboxylic acid ethyl ester

To a suspension of Zn (6.40 g, 97.5 mmol) in THF (120 mL) was added dropwise a solution of compound A2 (24.8 g, 81.1 mmol) in THF (24 mL) at rt, and then added THF (6 mL), 1,2-dibromoethan (0.30 ml) and trimethylsilylchloride (0.30 ml). After the reaction mixture was heated at 50-55° C. for 3 h, P(Ph)₃ (1.07 g, 4.08 mmol) and Pd(OAc)₂ (456 mg, 2.03 mmol) were added to the mixture with stirring. To a reaction mixture was added dropwise a solution of compound B1 (15.0 g, 40.6 mmol) in THF (135 mL) during 30 min period with stirring. After additional THF (15 mL) was added and heated at 50° C. for 30 min with stirring. After the reaction mixture was cooled to rt, the mixture was treated with 2N HCl (97.5 mL) and water (90 mL) and extracted with EtOAc (450 mL). The extract was washed with water (100 ml×2) and brine (100 mL), and dried over Na₂SO₄ The solvent was concentrated in vacuo to afford 41.6 g of crude compound A2-B1.

### Compound F: 4-Benzoyloxy-7-[4-fluoro-2-(2-trimethylsilanyl-ethoxymethoxymethyl)-benzyl]-1-methyl-2-oxo-1,2-dihydro-[1,5]naphthyridine-3-carboxylic acid ethyl ester

To a solution of crude compound A2-B1 (41.64 g) in CH₂Cl₂ (300 ml) were added Et₃N (11.5 ml, 82.5 mmol) and benzoyl chloride (7.0 ml, 60.3 mmol) at 0° C. After stirring for 1 h at rt, the reaction mixture was treated with 1N HCl (62 ml) and water (248 mL). The organic layer was separated, and washed with water (100 ml×2) and brine (100 ml). After dried over MgSO₄, the solvent was concentrated in vacuo to afford the crude product. The product mixture was purified by silicagel column chromatography (eluent: n-Hexane/EtOAc=3/2 v/v) to give 15.8 g of compound F (yield=62.4% from compound B1).

¹H NMR (CDCl₃) δ 8.35(1H, d, J=1.8 Hz), 8.20-8.24(2H, m), 7.50-7.70(3H, m), 7.43(1H, d, J=1.8 Hz), 6.95-7.18(3H, m), 4.69(2H, s), 4.54(2H, s), 4.32(2H, q, J=7.2 Hz), 4.17(2H, s), 3.58-3.64(5H, m), 1.18(3H, t, J=7.2 Hz), 0.91(2H, t, J=8.4 Hz), 0.00(9H, s).

### Compound G: 4-Benzoyloxy-7-(4-fluoro-2-hydroxymethyl-benzyl)-1-methyl-2-oxo-1,2-dihydro-[1,5]naphthyridine-3-carboxylic acid ethyl ester

To a solution of compound F (15.75 g, 25.37 mmol) in MeOH (120 mL) was added dropwise trimethylsilyl chloride (16.1 ml, 127 mmol) for 15 min. After addition of MeOH (6 mL), the mixture was stirring at 30-40° C. for 1 h. The mixture was treated with water (80 mL), the resulting mixture was stirring for 20 min at 0° C. The resulting precipitates were collected by filtration washing with water (40 mL×2) to afford 9.5 g of compound G (yield=76.6%) as a pale yellow crystal.

¹H NMR (CDCl₃) δ 8.34(1H, d, J=1.8 Hz), 8.20-8.23(2H, m), 7.50-7.70(3H, m), 7.46(1H, d, J=1.8 Hz), 6.95-7.19(3H, m), 4.63(2H, s), 4.32(2H, q, J=7.2 Hz), 4.17(2H, s), 3.63(3H, s), 1.18(3H, t, J=7.2 Hz).

### Compound H: 4-Benzoyloxy-7-(4-fluoro-2-formyl-benzyl)-1-methyl-2-oxo-1,2-dihydro-[1,5]naphthyridine-3-carboxylic acid ethyl ester

To a solution of compound G (9.25 g, 18.86 mmol) in CHCl₃ (270 mL) was added MnO₂ (27.75 g, 319 mmol), and the mixture was heated at reflux for 3 h. After cooling to rt, the insoluble materials were filtered off. The filtrate was concentrated in vacuo to afford the crude product, and the crude product was purified by recrystallization with CHCl₃/(ⁱPr)₂O to give 8.86 g of compounds H (yield=96.2%) as a colorless crystal.

¹H NMR (CDCl₃) δ 10.05(1H, s), 8.32(1H, d, J=1.8 Hz), 8.19-8.22(2H, m), 7.50-7.69(5H, m), 7.28-7.31(2H, m), 4.54(2H, s), 4.32(2H, q, J=7.2 Hz), 3.69(3H, s), 1.18(3H, t, J=7.2 Hz).

### Compound I: 4-Benzoyloxy-7-(2-carboxy-4-fluoro-benzyl)-1-methyl-2-oxo-1,2-dihydro-[1,5]naphthyridine-3-carboxylic acid ethyl ester

To a solution of NaClO₄ (2.24 g, 24.77 mmol) and sulfamide (2.41 g, 24.82 mmol) in water (100 mL) was added a solution of compound H (5.50 g, 11.26 mmol) in THF (350 mL) at rt, and the mixture was stirred for 1 h at 40° C. Additionally, to a reaction mixture was added a solution of NaClO₄ (1.12 g, 12.38 mmol) and sulfamide (1.21 g, 12.46 mmol) in water (50 mL), and stirred for 30 min at 40° C. The solvent was removed in vacuo to afford the crude residue, and the residue was treated with water (300 mL). The resulting precipitate was collected by filtration washing with water (100 mL) to give 5.33 g of compound I (yield=94.1%) as a colorless crystal.

¹H NMR (DMSO-d6) δ 8.34(1H, s), 8.10-8.13(2H, m), 7.98(1H, s), 7.79-7.84(1H, m), 7.59-7.67(3H, m), 7.36-7.50(2H, m), 4.52(2H, s), 4.22(2H, q, J=7.2 Hz), 3.64(3H, s), 1.08(3H, t, J=7.2 Hz).

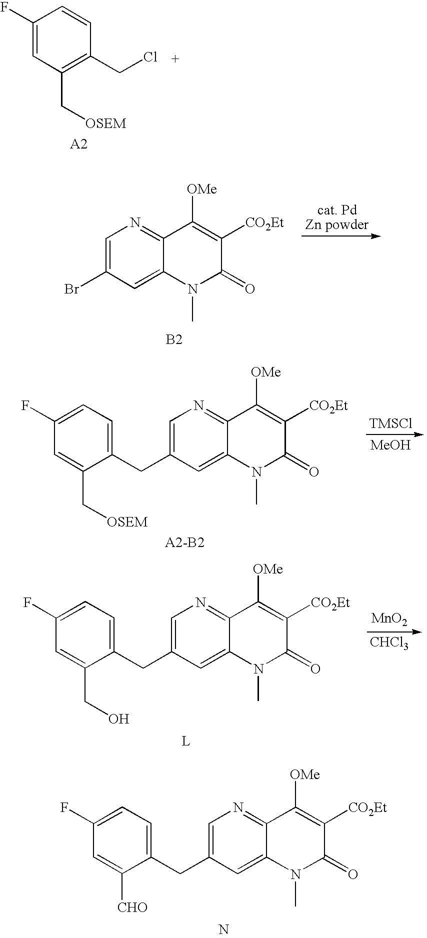

### Compound A2-B2: 7-[4-Fluoro-2-(2-trimethylsilanyl-ethoxymethoxymethyl)-benzyl]-4-methoxy-1-methyl-2-oxo-1,2-dihydro-[1,5]naphthyridine-3-carboxylic acid ethyl ester

To a suspension of Zn (10.3 g, 157 mmol) in THF (160 mL) was added dropwise compound A2 (31.9 g, 105 mmol) at rt, and then added successively THF (8 mL), 1,2-dibromoethane (0.40 ml) and trimethylsilyl chloride (0.40 ml). After the reaction mixture was heated at 55-60° C. for 60 min, the mixture was cooled to rt. To the reaction mixture were added P(Ph)₃ (1.38 g, 5.25 mmol), Pd(OAc)₂ (589 mg, 2.63 mmol) and compound B2 (16.1 g, 47.2 mmol), and heated at 60° C. for 30 min with stirring. After the reaction mixture was cooled to rt, the mixture was treated with sat. NaHCO₃ solution (100 mL) and EtOAc (200 mL). The insoluble materials were filtered off, and the filtrate was washed with water and brine, and dried over Na₂SO₄. The solvent was concentrated in vacuo to afford the crude product, and the crude product was purified by silicagel column chromatography (eluent: n-Hexane/EtOAc=1/2 v/v) to give 23.6 g of compound A2-B2 (yield=96.6%) as pale yellow oil.

¹H NMR (CDCl₃) δ 8.45(1H, d, J=1.8 Hz), 7.36(1H, d, J=1.8 Hz), 7.15-7.20(1H, m), 7.05-7.10(1H, m), 6.95-7.01(1H, m), 4.70(2H, s), 4.56(2H, s), 4.44(2H, q, J=7.2 Hz), 4.24(3H, s), 4.18(2H, s), 3.61(2H, t, J=8.4 Hz), 3.54(3H, s), 1.41(3H, t, J=7.2 Hz), 0.91(2H, t, J=8.4 Hz), 0.00(9H, s).

### Compound L: 7-(4-Fluoro-2-hydroxymethyl-benzyl)-4-methoxy-1-methyl-2-oxo-1,2-dihydro-[1,5]naphthyridine-3-carboxylic acid ethyl ester

To a solution of compound A2-B2 (23.6 g, 45.7 mmol) in MeOH (450 mL) was added dropwise trimethylsilyl chloride (29 ml, 228 mmol) at 0° C., and the mixture was stirred at rt for 30 min. Additional amount of trimethylsilyl chloride (29 ml, 228 mmol) was added, and stirred at rt for 15 min. The mixture was quenced with water (150 mL), the solvent was removed in vacuo. The residue was extracted twice with EtOAc and CHCl₃, the extract was washed with brine, dried over Na₂SO₄. The solvent was concentrated in vacuo, the resulting residue was purified by silicagel column chromatography (eluent: EtOAc) to give 15.0 g of compound L (yield=78.9%) as a yellow oil.

¹H NMR (CDCl₃) δ 8.42(1H, d, J=1.8 Hz), 7.38(1H, d, J=1.8 Hz), 7.17-7.21(1H, m), 7.06-7.11(1H, m), 6.93-6.99(1H, m), 4.65(2H, s), 4.43(2H, q, J=7.2 Hz), 4.23(3H, s), 4.18(2H, s), 3.52(3H, s), 1.40(3H, t, J=7.2 Hz).

### Compound N: 7-(4-Fluoro-2-formyl-benzyl)-4-methoxy-1-methyl-2-oxo-1,2-dihydro-[1,5]naphthyridine-3-carboxylic acid ethyl ester

To a solution of compound L (678 mg, 1.69 mmol) in CHCl₃ (30 mL) was added MnO₂ (1.47 g, 16.9 mmol), and the mixture was heated at reflux for 1 h. After allowing the reaction to cool to rt, the insoluble materials were filtered off. The filtrate was concentrated in vacuo to afford the crude product, and the crude product was purified by silicagel column chromatography (eluent: n-Hexane/EtOAc=1/2 v/v) to give 616 mg of compound N (yield=91.3%) as a yellow oil.

¹H NMR (CDCl₃) δ 10.08(1H,s), 8.43(1H, d, J=1.8 Hz), 7.55-7.58(1H, m), 7.46(1H, d, J=1.8 Hz), 7.30-7.33 (2H, m), 4.56(2H, s), 4.44(2H, q, J=7.2 Hz), 4.23(3H, s), 3.58(3H, s), 1.41(3H, t, J=7.2 Hz).

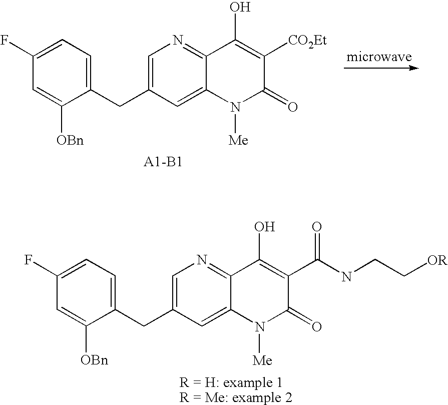

### EXAMPLE 1

### 7-(2-Benzyloxy-4-fluoro-benzyl)-4-hydroxy-1-methyl-2-oxo-1,2-dihydro-[1,5]naphthyridine-3-carboxylic acid hydroxymethyl-amide

A mixture of compound A1-B1 (176 mg, 0.38 mmol) in EtOH (2.2 mL) and 2-amino ethanol (51 μL, 0.84 mmol) was heated at 140° C. in a sealed tube in a microwave for 20 min. After cooling to rt, the resulting precipitate was collected by filtration. The crude product was purified by crystalization with with acetone/EtOH to give Example 1 (yield=55.0%).

715 (yield=80.0%).

m.p.: 166-167° C.

FAB-MS: m/z 478 (M+1)

¹H NMR (CDCl₃) δ 10.50(1H, br), 8.53(1H, d, J=1.8), 7.40-7.17(7H, m), 6.72-6.65(2H, m), 4.98(2H, s), 4.09(2H, s), 3.86(2H, t, J=5.1 Hz), 3.65(2H, m), 3.32(3H, s)

### EXAMPLE 2

### 7-(2-Benzyloxy-4-fluoro-benzyl)-4-hydroxy-1-methyl-2-oxo-1,2-dihydro-[1,5]naphthyridine-3-carboxylic acid methoxymethyl-amide

This compound was prepared in the similar manner as Example 1:

m.p.: 144-145° C.

FAB-MS: m/z 492 (M+1)

¹H NMR (CDCl₃) δ 10.36(1H, br), 8.54(1H, d, J=1.8), 7.38-7.17(7H, m), 6.73-6.65(2H, m), 4.98(2H, s), 4.10(2H, s), 3.70-3.57(4H, m), 3.42(3H, s), 3.32(3H, s)

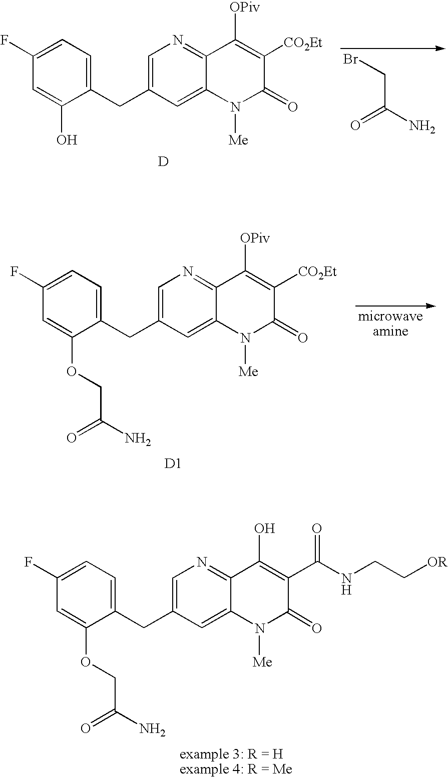

### Compound D1: 7-(2-Carbamoylmethoxy-4-fluoro-benzyl)-4-(2,2-dimethyl-propionyloxy)-1-methyl-2-oxo-1,2-dihydro-[1,5]naphthyridine-3-carboxylic acid ethyl ester

To a solution of compound D (500 mg, 1.14 mmol) in DMF (11 mL) were added potassium carbonate (188 mg, 1.36 mmol) and 2-Bromoacetamide (288 mg, 2.09 mmol) at rt, and stirred for 2 h. The mixture was treated with water and extracted with EtOAc. The combined organic layers were washed with water and dried over Na₂SO₄. The solvent was concentrated in vacuo to give 452 mg of compound D1 (yield=77.6%) as a colorless solid.

¹H NMR (DMSO-d6) δ 8.46(1H, s), 8.05(1H, s), 7.49(2H, bs), 7.38-7.33(1H, m), 6.81-6.77(2H, m), 4.51(2H, s), 4.27(2H, q, J=7.2 Hz), 4.16(2H, s), 3.62(3H, s), 1.31(9H, s), 1.25(3H, t, J=7.2 Hz).

### EXAMPLE 3

### 7-(2-Carbamoylmethoxy-4-fluoro-benzyl)-4-hydroxy-1-methyl-2-oxo-1,2-dihydro-[1,5]naphthyridine-3-carboxylic acid(2-hydroxy-ethyl)-amide

This compound was prepared from compound D1 in a manner similar to that described in Example 1:

m.p.: 243-244° C.

Elemental Analysis: C₂₁H₂₁F₁N₄O₆

Calcd (%): C, 56.75; H, 4.76; N, 12.61; F, 4.27.

Found (%): C, 56.49; H, 4.71; N, 12.46; F, 4.07.

¹H NMR (DMSO-d6) δ 10.44(1H, bs), 8.55(1H, s), 7.97(1H, s), 7.50(2H, bs), 7.37-7.33(1H, m), 6.82-6.78(1H, m), 4.93(1H, bs), 4.51(2H, s), 4.18(2H, s), 3.58-3.54(5H, m), 3.46-3.42(2H, m).

### EXAMPLE 4

### 7-(2-Carbamoylmethoxy-4-fluoro-benzyl)-4-hydroxy-1-methyl-2-oxo-1,2-dihydro-[1,5]naphthyridine-3-carboxylic acid(2-methoxy-ethyl)-amide

This compound was prepared from compound D1 in a manner similar to that described in Example 1:

m.p.: 219-220° C.

Elemental Analysis: C₂₂H₂₃F₁N₄O₆(H₂O)_{0.7}

Calcd (%): C, 56.09; H, 5.22; N, 11.89; F, 4.03.

Found (%): C, 56.17; H, 5.07; N, 11.74; F, 3.86.

¹H NMR (DMSO-d6) δ 10.41 (1H, bs), 8.56(1H, s), 7.99(1H, s), 7.50(2H, bs), 7.37-7.33(1H, m), 6.81-6.761H, m), 4.51(2H, s), 4.18(2H, s), 3.58(3H,s), 3.55-3.50(4H, m), 3.30(3H, s).

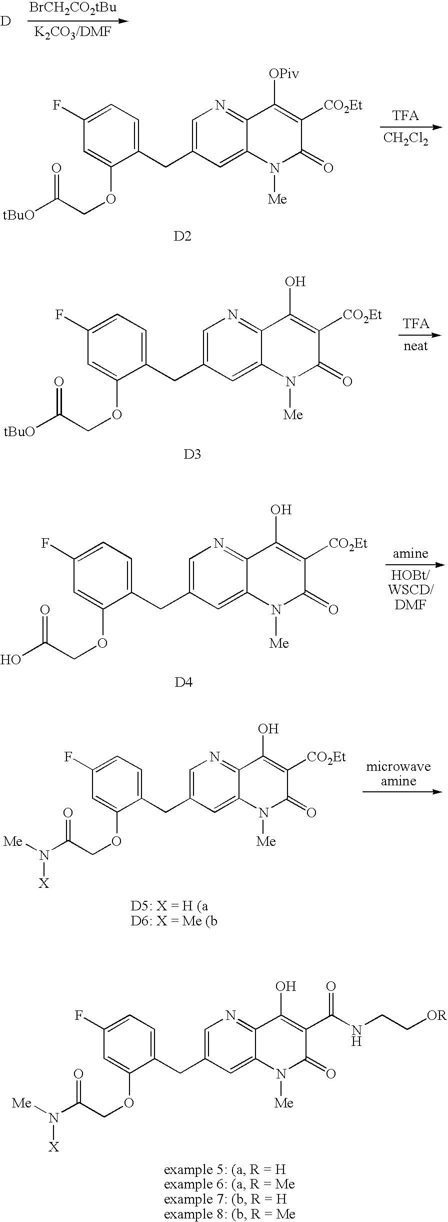

### Compound D2: 7-(2-tert-Butoxycarbonylmethoxy-4-fluoro-benzyl)-4-(2,2-dimethyl-propionyloxy)-1-methyl-2-oxo-1,2-dihydro-[1,5]naphthyridine-3-carboxylic acid ethyl ester

This compound was prepared from compound D in a manner similar to that described in compound D1.

¹H NMR (CDCl₃) δ 8.44(1H, s), 7.77(1H, s), 7.20-7.15(1H, m), 6.69-6.66(1H, m), 6.48-6.44(1H, m), 4.46(2H, s), 4.43(2H, q, J=7.2 Hz), 4.11(2H, s), 3.69(3H, s), 1.49(9H, s), 1.40(9H, s), 1.37(3H, t, J=7.2 Hz).

### Compound D3: 7-(2-tert-Butoxycarbonylmethoxy-4-fluoro-benzyl)-4-hydroxy-1-methyl-2-oxo-1,2-dihydro-[1,5]naphthyridine-3-carboxylic acid ethyl ester

To a solution of compound D2 (2.25 g, 3.94 mmol) in CH₂Cl₂ (20 mL) was added trifluor oacetic acid (3.9 ml, 51.2 mmol) at rt, and stirred for 1 h. The resulting mixture was quenched with ice/water, and extracted with CHCl₃. After the extract was washed with water, dried over Na₂SO₄, and then the solvent was concentrated in vacuo to afford the crude product. The crude product was washed to give 1.35 g of compound D3 (yield=75.0%) as a colorless solid.

¹H NMR (CDCl₃) δ 8.55(1H, s), 7.72(1H, s), 7.19-7.16(1H, m), 6.69-6.67(1H, m), 6.49-6.44(1H, m), 4.50(2H, q, J=7.2 Hz), 4.48(2H, s), 4.13(2H, s), 3.61(3H, s), 1.47(9H, s), 1.47(3H, t, J=7.2 Hz).

### Compound D4: 7-(2-Carboxymethoxy-4-fluoro-benzyl)-4-hydroxy-1-methyl-2-oxo-1,2-dihydro-[1,5]naphthyridine-3-carboxylic acid ethyl ester

A compound D3 (1.23 g, 2.53 mmol) was treated with trifluoroacetic acid (12 mL) at rt and stirred for 1 h. The resulting mixture was treated with ice/water and stirred for 10 min. The precipitated solid was collected by filtration, and the solid was recrystalized with MeOH/Et₂O to give 1.05 g of compound D4 (yield=96.0%) as a colorless solid.

1H NMR (CDCl₃) δ 8.55(1H, s), 7.95(1H, s), 7.42-7.37(1H, m), 6.88-6.74(2H, m), 4.77(2H, s), 4.24(2H, q, J=7.2 Hz), 4.11(2H, s), 3.51(3H, s), 1.25(3H, t, J=7.2 Hz).

### Compound D5: 7-(4-Fluoro-2-methylcarbamoylmethoxy-benzyl)-4-hydroxy-1-methyl-2-oxo-1,2-dihydro-[1,5]naphthyridine-3-carboxylic acid ethyl ester

A mixture of compound D4 (500 mg, 1.16 mmol), HOBt (188 mg, 1.39 mmol) and WSCD (333 mg, 1.74 mmol) in DMF (10 mL) was added methylamine (2N, 1.16 ml, 2.32 mmol) in THF at rt and stirred for 1 h. The resulting mixture was quenched with water (50 mL), and extracted with EtOAc. The extract was washed with water and brine, and then dried over Na₂SO₄. The solvent was concentrated in vacuo to give 438 mg of D5 (yield=85.0%) as a colorless solid.

¹H NMR (DMSO-d6) δ 8.50(1H, s), 7.97(1H, bs), 7.92(1H, s), 7.36-7.31(1H, m), 6.85-6.74(2H, m), 4.54(2H, s), 4.27-4.19(4H, m), 3.51(3H, s), 2.67(3H, d, J=4.8 Hz), 1.25(3H, t, J=7.2 Hz).

### Compound D6: 7-(2-Dimethylcarbamoylmethoxy-4-fluoro-benzyl)-4-hydroxy-1-methyl-2-oxo-1,2-dihydro-[1,5]naphthyridine-3-carboxylic acid ethyl ester

This compound was prepared from compound D4 in a manner similar to that described in compound D5.

¹H NMR (DMSO-d6) δ 8.57(1H, s), 8.02(1H, s), 7.38-7.36(1H, m), 6.90-6.73(2H, m), 4.90(2H, s), 4.23(2H, q, 7.2 Hz), 4.11(2H, s), 3.52(3H, s), 2.96(3H, s), 2.86(3H, s), 1.25(3H, t, J=7.2 Hz).

### EXAMPLE 5

### 7-(4-Fluoro-2-methylcarbamoylmethoy-benzyl)-4-hydroxy-1-methyl-2-oxo-1,2-dihydro-[1,5]naphthyridine-3-carboxylic acid(2-hydroxy-ethyl)-amide

This compound was prepared from compound D5 in a manner similar to that described in Example 1:

m.p.: 260-263° C.

Elemental Analysis: C₂₂H₂₃F₁N₄O₆

Calcd (%): C, 57.64; H, 5.06; N, 12.22; F, 4.14.

Found (%): C, 57.56; H, 5.01; N, 12.06; F, 3.94.

1H NMR (DMSO-d6) δ 10.42(1H, bs), 8.55(1H, s), 7.97(1H, s), 7.36-7.31(1H, m), 6.85-6.75(2H, m), 4.92(1H, t, J=5.1 Hz), 4.54(2H, s), 4.19(2H, s), 3.58-3.54(5H, m), 3.48-3.39(2H, m), 2.68(3H, d, J=4.8 Hz).

### EXAMPLE 6

### 7-(4-Fluoro-2-methylcarbamoylmethoxy-benzyl)-4-hydroxy-1-methyl-2-oxo-1,2-dihydro-[1,5]naphthyridine-3-carboxylic acid(2-methoxy-ethyl)-amide

This compound was prepared from compound D5 in a manner similar to that described in Example 1:

m.p.: 193-195° C.

Elemental Analysis: C₂₃H₂₅F₁N₄O₆(H₂O)_{0.2}

Calcd (%): C, 58.03; H, 5.38; N, 11.77; F, 3.99.

Found (%): C, 57.92; H, 5.34; N, 11.87; F, 3.80.

¹H NMR (DMSO-d6) δ 10.41(1H, bs), 8.55(1H, s), 7.97(1H, s), 7.36-7.33(1H, m), 6.84-6.77(2H, m), 4,54(2H, s), 4.19(2H, s), 3.58-3.50(7H, m), 3.30(3H, s), 2.68(3H, d, J=4.8 Hz).

### EXAMPLE 7

### 7-(2-Dimethylcarbamoylmethoxy-4-fluoro-benzyl)-4-hydroxy-1-methyl-2-oxo-1,2-dihydro-[1,5]naphthyridine-3-carboxylic acid(2-hydroxy-ethyl)-amide

This compound was prepared from compound D6 in a manner similar to that described in Example 1:

m.p.: 245-248° C.

¹H NMR (DMSO-d6) δ 10.44(1H, bs), 8.63(1H, s), 8.08(1H, s), 7.41-7.35(1H, m), 6.90-6.74(2H, m), 4.92(3H, bs), 4.12(2H, s), 3.59-3.54(5H, m), 3.46-3.43(2H, m), 2.96(3H, s), 2.87(3H, s).

### EXAMPLE 8

### 7-(2-Dimethylcarbamoylmethoxy-4-fluoro-benzyl)-4-hydroxy-1-methyl-2-oxo-1,2-dihydro-[1,5]naphthyridine-3-carboxylic acid(2-methoxy-ethyl)-amide

This compound was prepared from compound D6 in a manner similar to that described in Example 1:

m.p.: 224-226° C.

¹H NMR (DMSO-d6) δ 10.43(1H, bs), 8.63(1H, s), 8.07(1H, s), 7.40-7.35(1H, m), 6.90-6.71(2H, m), 4.91(2H, s), 4.12(2H, s), 3.59(3H, s), 3.55-3.50(4H, m), 3.30(3H, s), 2.96(3H, s), 2.86(3H, s).

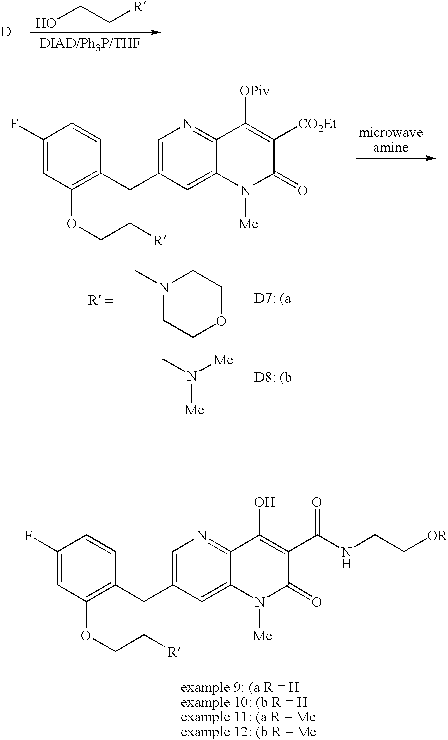

### Compound D7: 4-(2,2-Dimethyl-propionyloxy)-7-[4-fluoro-2-(2-morpholin-4-yl-ethoxy)-benzyl]-1-methyl-2-oxo-1,2-dihydro-[1,5]naphthyridine-3-carboxylic acid ethyl ester

To a solution of compound D (700 mg, 1.53 mmol) in THF (25 mL) were added Ph₃P (604 mg, 2.30 mmol) and 2-morpholine-4-yl-ethanol (0.279 ml, 2.30 mmol) at rt and diisopropyl azodicarboxylate in toluene (1.05 ml, 40 wt %) at 0° C., and then the mixture was stirred at rt for 1 h. The solvent was concentrated in vacuo to afford the crude product, the crude product was purified by silicagel column chromatography (eluent: EtOAc/MeOH=10/1 v/v) to give 803 mg of compound D7 as a colorless crystal.

¹H NMR (CDCl₃) δ 8.45(1H, d, J=1.8 Hz), 7.41(1H, d, J=1.8 Hz), 7.08-7.13 (1H, m), 6.59-6.68 (2H, m), 4.40(2H, q, J=7.2 Hz), 4.05(2H, t, J=5.7 Hz), 4.03(2H, s), 3.69-3.72 (4H, m), 3.63(3H, s), 2.73(2H, t, J=5.7 Hz), 2.52-2.55 (4H, m), 1.41(9H, s), 1.37(3H, t, J=7.2 Hz).

### Compound D8: 7-[2-(2-Dimethylamino-ethoxy)-4-fluoro-benzyl]-4-(2,2-dimethyl-propionyloxy)-1-methyl-2-oxo-1,2-dihydro-[1,5]naphthyridine-3-carboxylic acid ethyl ester

This compound was prepared from compound D in a manner similar to that described in compound D7 and was obtained at 81.1% yield.

¹H NMR (CDCl₃) δ 8.43(1H, d, J=1.8 Hz), 7.47(1H, d, J=1.8 Hz), 7.08-7.13 (1H, m), 6.59-6.68 (2H, m), 4.40(2H, q, J=7.2 Hz), 4.05(2H, s), 4.04(2H, t, J=5.7 Hz), 3.64(3H, s), 2.71(2H, t, J=5.7 Hz), 2.32 (6H, s), 1.41(9H, s), 1.37(3H, t, J=7.2 Hz).

### EXAMPLE 9

### 7-[4-Fluoro-2-(2-morpholin-4-yl-ethoxy)-benzyl]-4-hydroxy-1-methyl-2-oxo-1,2-dihydro-[1,5]naphthyridine-3-carboxylic acid(2-hydroxy-ethyl)-amide

This compound was prepared from compound D7 in a manner similar to that described in Example 1 and was obtained at 77.7% yield:

m.p.: 161-162° C.

Elemental Analysis: C₂₅H₂₉F₁N₄O₆

Calcd (%): C, 59.99; H, 5.84; N, 11.19; F, 3.80.

Found (%): C, 60.04; H, 5.90; N, 11.26; F, 3.63.

¹H NMR (CDCl₃) δ 10.49(1H, bs), 8.60(1H, d, J=1.8 Hz), 7.42(1H, d, J=1.8 Hz), 7.10-7.15 (1H, m), 6.61-6.70 (2H, m), 4.08(4H, bs), 3.86(2H, bs), 3.70(4H, bs), 3.59(3H, s), 2.79(2H, bs), 2.55(4H, bs), 2.31 (2H, bs).

### EXAMPLE 10

### 7-[2-(2-Dimethylamino-ethoxy)-4-fluoro-benzyl]-4-hydroxy-1-methyl-2-oxo-1,2-dihydro-[1,5]naphthyridine-3-carboxylic acid(2-hydroxy-ethyl)-amide

This compound was prepared from compound D8 in a manner similar to that described in Example 1 and was obtained at 41.7% yield:

m.p.: 108-109° C.

Elemental Analysis: C₂₃H₂₇F₁N₄O₅

Calcd (%): C, 60.25; H, 5.94; N, 12.22; F, 4.14.

Found (%): C, 59.88; H, 5.97; N, 12.14; F, 3.90.

¹H NMR (CDCl₃) δ 10.50(1H, bs), 8.60(1H, d, J=1.8 Hz), 7.47(1H, d, J=1.8 Hz), 7.10-7.15 (1H, m), 6.61-6.68(2H, m), 4.06-4.10(4H, m), 3.85-3.88(2H, m), 3.52-3.68(5H, m), 2.75-2.79(2H, m), 2.35(6H, s).

### EXAMPLE 11

### 7-[4-Fluoro-2-(2-morpholin-4-yl-ethoxy)-benzyl]-4-hydroxy-1-methyl-2-oxo-1,2-dihydro-[1,5]naphthyridine-3-carboxylic acid(2-methoxy-ethyl)-amide

This compound was prepared from compound D7 in a manner similar to that described in Example 1 and was obtained at 63.2% yield:

m.p.: 167-168° C.

Elemental Analysis: C₂₆H₃₁F₁N₄O₆

Calcd (%): C, 60.69; H, 6.07; N, 10.89; F, 3.69.

Found (%): C, 60.48; H, 6.03; N, 10.87; F, 3.40.

¹H NMR (CDCl₃) δ 10.37(1H, bs), 8.60(1H, d, J=1.8 Hz), 7.41(1H, d, J=1.8 Hz), 7.10-7.15 (1H, m), 6.61-6.68 (2H, m), 4.07(4H, bs), 3.57-3.70(11H, m), 3.42(3H, s), 2.76(2H, bs), 2.52(4H, bs).

### EXAMPLE 12

### 7-[2-(2-Dimethylamino-ethoxy)-4-fluoro-benzyl]-4-hydroxy-1-methyl-2-oxo-1,2-dihydro-[1,5]naphthyridine-3-carboxylic acid(2-methoxy-ethyl)-amide

This compound was prepared from compound D8 in a manner similar to that described in Example 1 and was obtained at 28.3% yield:

m.p.: 131-133° C.

Elemental Analysis: C₂₄H₂₉F₁N₄O₅

Calcd (%): C, 61.01; H, 6.19; N, 11.86; F, 4.02.

Found (%): C, 60.12; H, 6.09; N, 11.66; F, 3.91.

¹H NMR (CDCl₃) δ 10.37(1H, bs), 8.59(1H, d, J=1.8 Hz), 7.46(1H, d, J=1.8 Hz), 7.11-7.16 (1H, m), 6.62-6.70(2H, m), 4.16(2H, bs), 4.11(2H, s), 3.58-3.67(7H, m), 3.42(3H, s), 2.90(2H, bs), 2.44(6H, s).

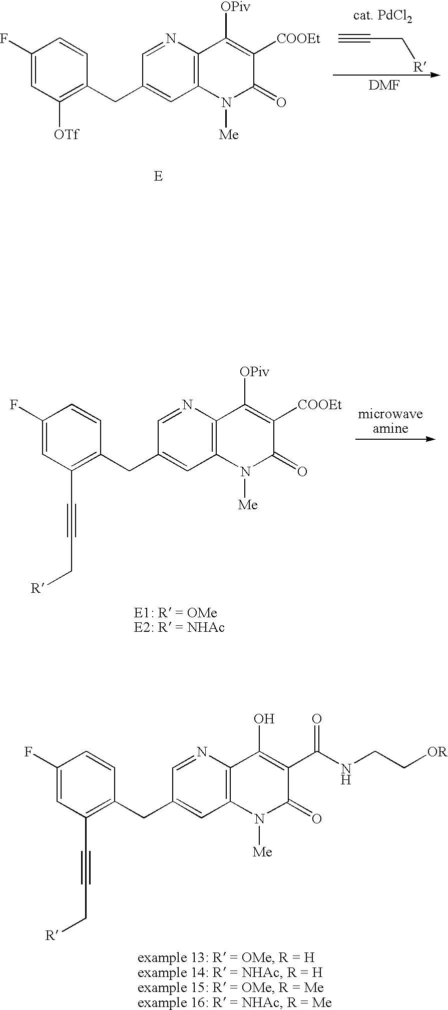

### Compound E1: 4-(2,2-Dimethyl-propionyloxy)-7-[4-fluoro-2-(3-methoxy-prop-1-ynyl)-benzyl]-1-methyl-2-oxo-1,2-dihydro-[1,5]naphthyridine-3-carboxylic acid ethyl ester

To a suspension of compound E (600 mg, 1.02 mmol), Bis(triphenylphosphine)palladium(II)chloride (36 mg, 0.051 mmol) and CuI (19 mg, 0.10 mmol) in DMF (10 mL) were added methylpropargyl ether (0.864 mL, 8.50 mmol) and Et₃N (1.44 mL, 8.50 mmol) at rt. The mixture was heated at 120° C. in a sealed tube in a microwave for 15 min. The mixture was treated with water, and extracted with EtOAc. The combined layers were washed with water and dried over Na₂SO₄. The solvent was concentrated in vacuo, and the product mixture was purified by silicagel column chromatography (eluent: EtOAc/n-Hexane=1/3 v/v) to afford 261 mg of compound E-1 (yield=50.0%) as an orange form.

¹H NMR (CDCl₃) δ 8.40 (d, J=1.8Hz, 1H), 7.51 (s, 1H), 7.21-7.15 (m, 2H), 7.05-6.99 (dt, J=8.1 Hz, 2.7 Hz, 1H), 4.40 (q, 7.2 Hz, 2H), 4.29 (s, 2H), 4.24 (s, 2H), 3.64 (s, 3H), 3.37 (s, 3H), 1.41 (s, 9H), 1.37 (t, J=7.2 Hz, 3H).

### Compound E2: 7-[2-(3-Acetylamino-prop-1-ynyl)-4-fluoro-benzyl]-4-(2,2-dimethyl-propionyloxy)-1-methyl-2-oxo-1,2-dihydro-[1,5]naphthyridine-3-carboxylic acid ethyl ester

This compound was prepared from compound E in a manner similar to that described in compound E1 and was obtained at 58% yield:

¹H NMR (CDCl₃) δ8.38 (d, J=1.8 Hz, 1H), 7.41 (s, 1H), 7.20-7.12 (m, 2H), 7.01 (dt, J=8.10 Hz, 2.7 Hz, 1H), 4.40 (q, J=7.2 Hz, 2H), 4.21 (s, 2H), 4.17 (d, J=5.7 Hz, 2H), 3.60 (s, 3H), 1.98 (s, 3H), 1.41 (s, 9H), 1.36 (t, J=7.2 Hz, 3H).

### EXAMPLE 13

### 7-[4-Fluoro-2-(3-methoxy-prop-1-ynyl)-benzyl]-4-hydroxy-1-methyl-2-oxo-1,2-dihydro-[1,5]naphthyridine-3-carboxylic acid(2-hydroxy-ethyl)-amide

This compound was prepared from compound E1 in a manner similar to that described in Example 1 and was obtained at 58% yield:

m.p.: 188-189° C.

¹H NMR (DMSO-d₆) δ 10.43 (t, J=5.4 Hz, 1H), 8.49 (s, 1H), 7.94 (dd, J=8.4 Hz, 5.7 Hz, 1H), 7.35 (dd, J=9.6 Hz, 3.0 Hz, 1H), 7.25 (dt, J=8.7 H, 3.0 Hz, 1H), 4.59 (s, 1H), 4.40 (s, 3H), 4.30 (s, 3H), 3.56 (s, 3H), 3.50-3.40 (m, 4H), 3.30 (s, 3H).

### EXAMPLE 14

### 7-[2-(3-Acetylamino-prop-1-ynyl)-4-fluoro-benzyl]-4-hydroxy-1-methyl-2-oxo-1,2-dihydro-[1,5]naphthyridine-3-carboxylic acid(2-hydroxy-ethyl)-amide

This compound was prepared from compound E2 in a manner similar to that described in Example 1 and was obtained at 58% yield:

m.p.: 220-221° C.

¹H NMR (DMSO-d₆) δ10.44 (s, 1H), 8.53 (s, 1H), 8.43 (t, J=5.1 Hz, 1H), 7.97 (s, 1H), 7.51 (dd, J=8.7 Hz, 6.0 Hz, 1H), 7.29-7.19 (m, 2H), 4.95 (t, J=4.80 Hz, 1H), 4.27 (s, 2H), 4.15 (d, J=5.4 Hz, 2H), 3.59 (s, 3H), 3.59-3.41 (m, 4H), 1.84 (s, 3H).

### EXAMPLE 15

### 7-[4-Fluoro-2-(3-methoxy-prop-1-ynyl)-benzyl]-4-hydroxy-1-methyl-2-oxo-1,2-dihydro-[1,5]naphthyridine-3-carboxylic acid(2-methoxy-ethyl)-amide

This compound was prepared from E1 in a manner similar to that described in Example 1 and was obtained at 62% yield:

m.p.: 179-180° C.

Elemental Analysis: C₂₄H₂₄FN₃O₅

Calcd (%): C, 63.57; H, 5.33; N, 9.27; F, 4.19.

Found (%): C, 63.57; H, 5.31; N, 9.24; F, 3.97.

¹H NMR (DMSO-d₆) δ10.24 (s, 1H), 8.34 (s, 1H), 7.79 (s, 3H), 7.32 (dd, J=8.7 Hz, 6.0 Hz, 1H), 7.18 (dd, J=9.3 Hz, 2.7 Hz, 1H), 7.08 (dt, J=8.7 Hz, 3.0 Hz, 1H), 4.22 (s, 2H), 4.13 (s, 2H), 3.40 (s, 3H), 3.40-3.30 (m, 4H), 3.18 (s, 3H), 3.13 (s, 3H).

### EXAMPLE 16

### 7-[2-(3-Acetylamino-prop-1-ynyl)-4-fluoro-benzyl]-4-hydroxy-1-methyl-2-oxo-1,2-dihydro-[1,5]naphthyridine-3-carboxylic acid(2-methoxy-ethyl)-amide

This compound was prepared from compound E2 in a manner similar to that described in Example 1 and was obtained at 58% yield:

m.p.: 224-225° C.

Elemental Analysis: C₂₅H₂₅FN₄O₅

Calcd (%): C, 62.49; H, 5.24; N, 11.66; F, 3.95.

Found (%): C, 62.14; H, 5.29; N, 11.75; F, 3.69.

¹H NMR (DMSO-d₆) δ10.42 (s, 1H), 8.53 (s, 1H), 8.43 (t, J=4.8 Hz, 1H), 7.98 (s, 1H), 7.51 (dd, J=8.7 Hz, 6.0 Hz, 1H), 7.29-7.19 (m, 2H), 4.28 (s, 2H), 4.15 (d, J=5.4 Hz, 2H), 3.59 (s, 3H), 3.56-3.30 (m, 4H), 3.30 (s, 3H), 1.84 (s, 3H).

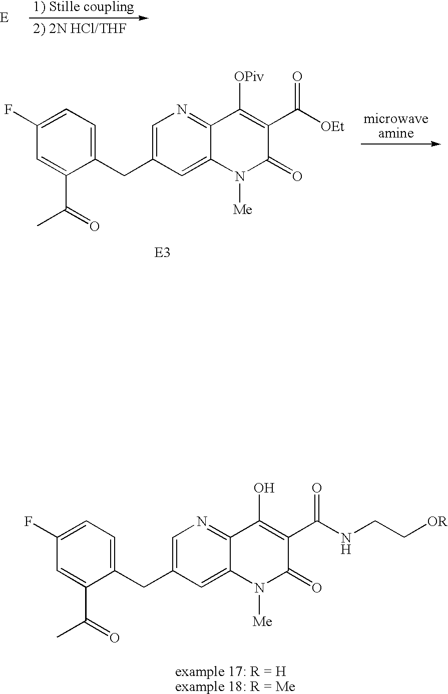

### Compound E3: 7-(2-Acetyl-4-fluoro-benzyl)-4-(2,2-dimethyl-propionyloxy)-1-methyl-2-oxo-1,2-dihydro-[1,5]naphthyridine-3-carboxylic acid ethyl ester

A mixture of compound E (589 mg, 1 mmol), tributhyltin(1-ethoxyvinyl) (0.51 mL, 1.5 mmol), Bis(triphenylphosphine)palladium(II)chloride (70 mg, 0.10 mmol) and LiCl (130 mg, 3.07 mmol) in DMF (10 mL) was heated at 120° C. in a sealed tube in a microwave for 15 min. After cooling, the solvent was concentrated in vacuo, and the residue was treated with THF (20 mL) and 2N HCl (2 mL, 4 mmol) with stirring. After stirring for 1 h, the mixture was poured into ice/water and extracted with EtOAc and washed with water. The extract was dried over Na₂SO₄ and the solvent was concentrated in vacuo to afford the crude product. The crude product was crystallized with (ⁱPr)₂O, and the crystal was washed twice with (ⁱPr)₂O to give 355 mg of crude crystal. The crude crystal was purified by silicagel column chromatography (eluent: EtOAc/n-Hexane=1/3 to 2/1 v/v) to give 198 mg of compound E-3 (yield=41.0%) as an orange form.

¹H NMR (CDCl₃) δ 8.32(1H, d, J=1.8 Hz), 7.53(1H, d, J=1.8 Hz), 7.50-7.46(1H, dd, J=2.4, 9.0 Hz), 7.31-7.17(2H, m), 4.39(2H, quart, J=7.2 Hz), 3.64(3H, s), 2.53(3H, s), 1.40(9H, s), 1.37(3H, trip, J=7.2 Hz).

### EXAMPLE 17

### 7-(2-Acetyl-4-fluoro-benzyl)-4-hydroxy-1-methyl-2-oxo-1,2-dihydro-[1,5]naphthyridine-3-carboxylic acid(2-hydroxy-ethyl)-amide

This compound was prepared from compound E3 in a manner similar to that described in Example 1 and was obtained at 66.5% yield:

m.p.: 212-215° C.

Elemental Analysis: C₂₁H₂₀FN₃O₅

Calcd (%): C, 61.01; H, 4.88; N, 10.16; F, 4.60.

Found (%): C, 60.89; H, 4.82; N, 10.23; F, 4.39.

¹H NMR (DMSO-d6) δ 10.40(1H, m), 8.40(1H, d, J=1.2 Hz), 7.82(1H, s), 7.76(1H, dd, J=2.7, 9.6 Hz), 7.45-7.33(2H, m), 4.92(1H, trip, J=5.1 Hz), 4.34(2H, s), 3.53(3H, s), 3.56-3.28(4H, m), 2.54(3H, s).

### EXAMPLE 18

### 7-(2-Acetyl-4-fluoro-benzyl)-4-hydroxy-1-methyl-2-oxo-1,2-dihydro-[1,5]naphthyridine-3-carboxylic acid(2-methoxy-ethy)-amide

This compound was prepared from compound E3 in a manner similar to that described in Example 1 and was obtained at 73.7% yield:

m.p.: 217° C.

Elemental Analysis: C₂₂H₂₂FN₃O₅

Calcd (%): C, 61.82; H, 5.19; N, 9.83; F, 4.44.

Found (%): C, 61.59; H, 5.13; N, 9.84; F, 4.30.

¹H NMR (CDCl₃) δ 10.37(1H, m), 8.49(1H, D, J=1.5 Hz), 7.57(1H, d, J=1.5 Hz), 7.49(1H, dd, J=2.7, 9.0 Hz), 7.32-7.18(2H, m), 4.40(2H, s), 3.69-3.57(4H, m), 3.61(3H, s), 3.42(3H, s), 2.54(3H, s).

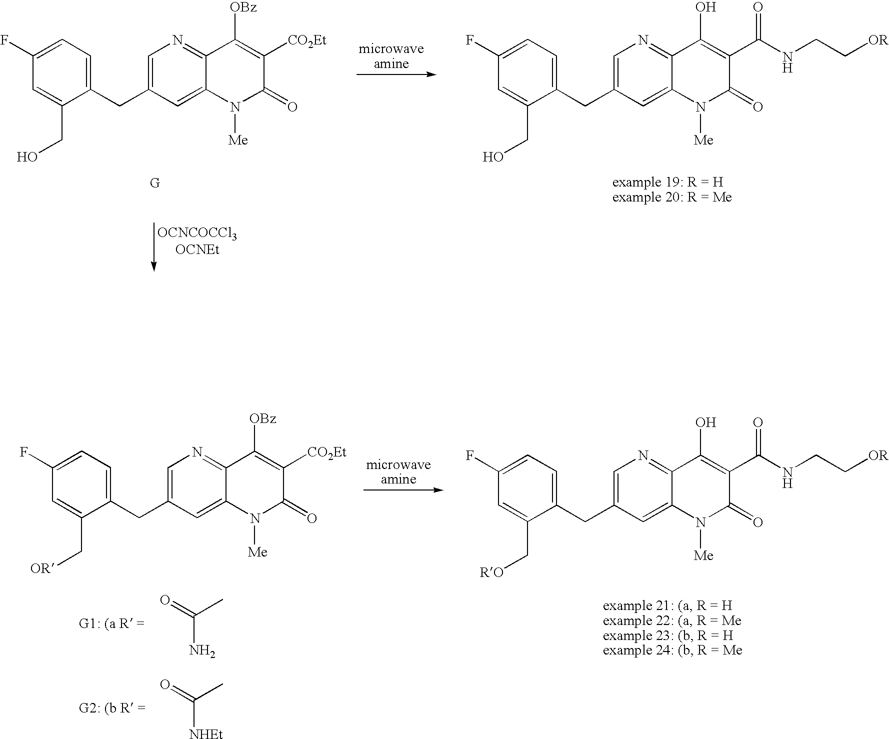

### EXAMPLE 19

### 7-(4-Fluoro-2-hydroxymethyl-benzyl)-4-hydroxy-1-methyl-2-oxo-1,2-dihydro-[1,5]naphthyridine-3-carboxylic acid(2-hydroxy-ethyl)-amide

This compound was prepared from compound G in a manner similar to that described in Example 1:

m.p.: 190-192° C.

Elemental Analysis: C₂₀H₂₀F₁N₃O₅

Calcd (%): C, 59.85; H, 5.02; N, 10.47; F, 4.73.

Found (%): C, 59.51; H, 5.07; N, 10.48; F, 4.45.

¹H NMR (DMSO-d6) δ 10.42(1H, bs), 8.46(1H, s), 7.90(1H, s), 7.26-7.19(2H, m), 7.08-7.01(1H, m), 5.38(1H, t, J=5.5 Hz), 4.95(1H, t, J=5.0 Hz), 4.55(2H, d, J=5.3 Hz), 4.18(2H, s), 3.60-3.52(5H, m), 3.48-3.40(2H, m).

### EXAMPLE 20

### 7-(4-Fluoro-2-hydroxymethyl-benzyl)-4-hydroxy-1-methyl-2-oxo-1,2-dihydro-[1,5]naphthyridine-3-carboxylic acid(2-methoxy-ethyl)-amide

This compound was prepared from compound G in a manner similar to that described in Example 1:

m.p.: 193-195° C.

Elemental Analysis: C₂₁H₂₂F₁N₃O₅

Calcd (%): C, 60.72; H, 5.34; N, 10.12; F, 4.57.

Found (%): C, 60.68; H, 5.28; N, 10.12; F, 4.27.

¹H NMR (DMSO-d6) δ 10.42(1H, bs), 8.46(1H, s), 7.90(1H, s), 7.26-7.19(2H, m), 7.08-7.01(1H, m), 5.37(1H, t, J=5.4 Hz), 4.55(2H, d, J=5.3 Hz), 4.18(2H, s), 3.60-3.45(7H, m), 3.31(3H, s).

### Compound G1: 4-Benzoyloxy-7-(2-carbamoyloxymethyl-4-fluoro-benzyl)-1-methyl-2-oxo-1,2-dihydro-[1,5]naphthyridine-3-carboxylic acid ethyl ester

A solution of compound G (150 mg, 0.306 mmol) in THF (3 mL) was added trichloroacetyl isocyanate (67.5 μL, 0.551 mmol) at −20° C. and stirred at 0° C. for 10 min. The resulting mixture was poured into EtOAc-ice/water and extracted with EtOAc. The extract was washed with brine and dried over Na₂SO₄, then the solvent was removed in vacuo to afford the crude intermediate. The crude intermediate was dissolved in CHCl₃ (4 mL), and then it was treated with excess of Al₂O₃ for 1 h with stirring. The reaction mixture was passed through a pad of celite, and the pad was washed with CHCl₃/MeOH (4/1 v/v). The filtrate was concentrated under reduced pressure to afford the crystalline solid, and the solid was washed with Et₂O to give 140 mg of compound G1 (yield=86.0%) as a colorless crystal.

¹H NMR (DMSO-d6) δ 8.37(1H, s), 8.13-8.11(2H, m), 7.96(1H, s), 7.83-7.79(1H, m), 7.67-7.62(2H, m), 7.29-7.10(3H, m), 6.67(2H, bs), 5.06(2H, s), 4.24-4.22(4H, m), 3.65(3H, s), 1.09(3H, t, J=7.2 Hz).

### EXAMPLE 21

### Carbamic acid 5-fluoro-2-[8-hydroxy-7-(2-hydroxy-ethylcarbamoyl)-5-methyl-6-oxo-5,6-dihydro-[1,5]naphthyridin-3-ylmethyl]-benzyl ester

This compound was prepared from compound G1 in a manner similar to that described in Example 1:

m.p.: 238-240° C.

Elemental Analysis: C₂₁H₂₁F₁N₄O₆(H₂O)_{0.3}

Calcd (%): C, 56.07; H, 4.84; N, 12.46; F, 4.22.

Found (%): C, 56.29; H, 4.89; N, 12.16; F, 3.84.

¹H NMR (DMSO-d6) δ 10.43(1H, bs), 8.46(1H, s), 7.86(1H, s), 7.28-7.14(3H, m), 6.63(2H, bs), 5.06(2H, s), 4.93(1H, t, J=5.1 Hz), 4.23(2H, s), 4.18(2H, s), 3.57-3.54(5H, m), 3.46-3.43(2H, m).

### EXAMPLE 22

### Carbamic acid 5-fluoro-2-[8-hydroxy-7-(2-methoxy-ethylcarbamoyl)-5-methyl-6-oxo-5,6-dihydro-[1,5]naphthyridin-3-ylmethyl]-benzyl ester

This compound was prepared from compound G1 in a manner similar to that described in Example 1:

m.p.: 180-183° C.

Elemental Analysis: C₂₂H₂₃F₁N₄O₆

Calcd (%): C, 57.64; H, 5.06; N, 12.22; F, 4.14.

Found (%): C, 57.36; H, 5.10; N, 11.96; F, 3.87.

¹H NMR (DMSO-d6) δ 8.46(1H, s), 7.86(1H, s), 7.27-7.14(3H, m), 6.64(2H, bs), 5.06(2H, s), 4.22(2H, s), 3.56-3.51(7H, m), 3.30(3H, s).

### Compound G2: 4-Benzoyloxy-7-(2-ethylcarbamoyloxymethyl-4-fluoro-benzyl)-1-methyl-2-oxo-1,2-dihydro-[1,5]naphthyridine-3-carboxylic acid ethyl ester

A solution of compound G (250 mg, 0.510 mmol) in THF (5 mL) was added ethylisocyanate (60.5 μL, 0.765 mmol) at 0° C., and then tributyltinoxide (2 drops) was added to the mixture at rt with stirring. The mixture was stirred for 30 min and heated at 40° C. for 1 h. The resulting mixture was poured into EtOAc/water and extracted with EtOAc. After the extract was washed with brine and dried over Na₂SO₄, the solvent was concentrated in vacuo to afford the crude product. The product mixture was purified by silicagel column chromatography (eluent: n-Hexan/EtOAc=2/1 v/v) to give 254 mg of compound G2 (yield=89.0%) as a colorless form.

¹H NMR (CDCl₃) δ 8.37(1H, s), 8.21-8.18(2H, m), 7.69-7.66(1H, m), 7.54-7.45(3H, m), 7.19-7.01(3H, m), 5.01(2H, s), 4.50(1H, bs), 4.32(2H, q, J=7.2 Hz), 4.17(2H, s), 3.67(3H, s), 3.16-3.06(2H, m), 1.18(3H, t, J=7.2 Hz), 1.05(3H, t, J=7.2 Hz).

### EXAMPLE 23

### Ethyl-carbamic acid 5-fluoro-2-[8-hydroxy-7-(2-hydroxy-ethylcarbamoyl)-5-methyl-6-oxo-5,6-dihydro-[1,5]naphthyridin-3-ylmethyl]-benzyl ester

This compound was prepared from compound G2 in a manner similar to that described in Example 1:

m.p.: 180-182° C.

Elemental Analysis: C₂₃H₂₅F₁N₄O₆

Calcd (%): C, 58.47; H, 5.33; N, 11.86; F, 4.02.

Found (%): C, 58.46; H, 5.38; N, 11.83; F, 3.90.

¹H NMR (DMSO-d6) δ 10.25(1H, bs), 8.22(1H, s), 7.61(1H, s), 7.31-7.11(3H, m), 4.90(2H, s), 4.04(2H, s), 3.39-3.36(5H, m), 3.25-3.19(2H, m), 2.82-2.78(2H, m), 4.37(1H, t, J=5.4 Hz), 0.80(3H, t, J=7.4 Hz).

### EXAMPLE 24

### Ethyl-carbamic acid 5-fluoro-2-[8-hydroxy-7-(2-methoxy-ethylcarbamoyl)-5-methyl-6-oxo-5,6-dihydro-[1,5]naphthyridin-3-ylmethyl]-benzyl ester

This compound was prepared from compound G2 in a manner similar to that described in Example 1:

m.p.: 157-159° C.

Elemental Analysis: C₂₄H₂₇F₁N₄O₆

Calcd (%): C, 59.25; H, 5.59; N, 11.52; F, 3.91.

Found (%): C, 59.17; H, 5.52; N, 11.50; F, 3.60.

¹H NMR (DMSO-d6) δ 10.41(1H, bs), 8.45(1H, s), 7.86(1H, s), 7.35-7.09(3H, m), 5.08(2H, s), 4.23(2H, s), 3.56-3.51(7H, m), 2.98-2.94(2H, m), 0.97(3H, t, J=7.4 Hz).

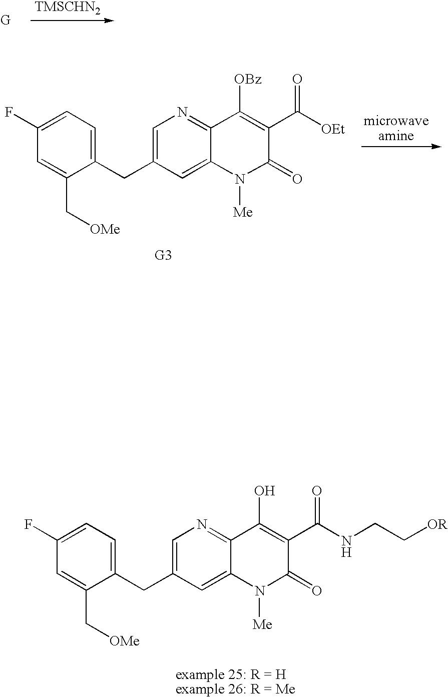

### Compound G3: 4-Benzoyloxy-7-(4-fluoro-2-methoxymethyl-benzyl)-1-methyl-2-oxo-1,2-dihydro-[1,5]naphthyridine-3-carboxylic acid ethyl ester

To a solution of compound G (815.0 mg, 1.66 mmol) in CH₂Cl₂ (8 mL) were added 48% HBF₄ solution and 2M TMSCH₂N₂ in n-hexane (4.99 ml, 9.96 mmol) at 0° C., and the reaction mixture was stirred for 5 h. The mixture was quenched with water (5 mL), and extracted with CHCl₃, dried over Na₂SO₄. The solvent was concentrated in vacuo, and the residue was purified by silicagel column chromatography (eluent: CHCl₃) to give 329 mg of compound G3 (yield=39.0% ) as a colorless solid.

¹H NMR (CDCl₃) δ 8.22 (2H, d, J=7.2 Hz), 7.67 (1H, dd, J=7.5, 7.5 Hz), 7.53 (2H, dd, J=8.1, 7.5), 7.44 (1H, s), 7.27-7.10 (4H, m), 4.37(2H, s), 4.32(2H, q, J=7.2 Hz), 4.15(2H, s), 3.63(3H,s), 3.36(3H, s), 1.18 (3H, t, J=7.2 Hz).

### EXAMPLE 25

### 7-(4-Fluoro-2-methoxymethyl-benzyl)-4-hydroxy-1-methyl-2-oxo-1,2-dihydro-[1,5]naphthyridine-3-carboxylic acid(2-hydroxy-ethyl)-amide

This compound was prepared from compound G3 in a manner similar to that described in Example 1:

m.p.: 162-164° C.

¹H NMR (CDCl₃) δ 10.49(1H, bs), 8.58(1H, s), 7.40(1H, s), 7.12-7.09(2H, m), 7.03-7.00(1H, m), 4.39(2H, s), 4.20(2H, s), 3.87(2H, t, J=4.8 Hz), 3.68-3.62(2H, m), 3.55 (3H, s), 3.37(3H, s), 2.87 (1H, brs).

### EXAMPLE 26

### 7-(2-Carbamoyl-4-fluoro-benzyl)-4-hydroxy-1-methyl-2-oxo-1,2-dihydro-[1,5]naphthyridine-3-carboxylic acid(2-methoxy-ethyl)-amide

This compound was prepared from compound G3 in a manner similar to that described in Example 1:

m.p.: 171-172° C.

¹H NMR (CDCl₃) δ 10.35(1H, bs), 8.59(1H, s), 7.41(1H, s), 7.12-7.09(2H, m), 7.03-6.98(1H, m), 4.39(2H, s), 4.19(2H, s), 3.66(2H, t, J=5.4 Hz), 3.61-3.57(2H, m), 3.55 (3H, s), 3.42(3H, s), 3.67(3H, s).

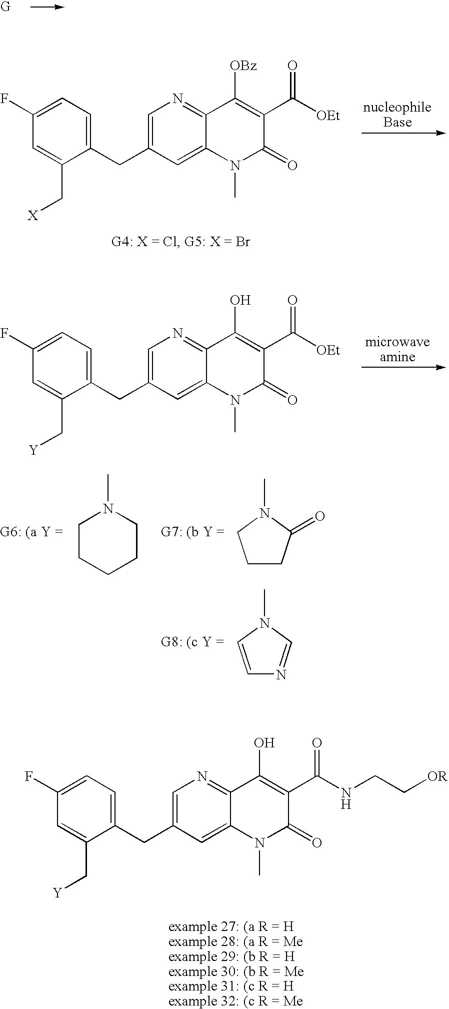

### Compound G4: 4-Benzoyloxy-7-(2-chloromethyl-4-fluoro-benzyl)-1-methyl-2-oxo-1,2-dihydro-[1,5]naphthyridine-3-carboxylic acid ethyl ester

A mixture of compound G (1.0 g, 2.04 mmol) was treated with thionyl chloride (178 □L, 2.45 mmol) at 0° C. under N₂ atmosphere. The resulting mixture was stirred at rt for 5 min and was treated with ice/water. The product mixture was extracted three times with EtOAc, and the combined organic layer was washed with sat. NaHCO₃ solution, water and then brine. After dried over Na₂SO₄, the solvent was concentrated in vacuo to give pale yellow solid. The resulting solid was washed with diisopropylether to give 998 mg of compound G4 as a colorless powder.

¹H NMR (CDCl₃) δ 8.35(1H, s), 8.22(2H, d, J=7.2 Hz), 7.67(1H, t, J=7.2 Hz), 7.53(2H, t, J=7.8 Hz), 7.44(1H, s), 7.15-6.99(3H, m), 4.50(2H, s), 4.33(2H, q, J=7.2 Hz), 4.25(2H, s), 3.65(3H, s), 1.18(3H, t, 7.2 Hz)

### Compound G5: 4-Benzoyloxy-7-(2-bromomethyl-4-fluoro-benzyl)-1-methyl-2-oxo-1,2-dihydro-[1,5]naphthyridine-3-carboxylic acid ethyl ester

To a mixture of compound G (1.5 g, 3.06 mmol) and carbon tetrabromide (3.0 g, 9.17 mmol) in CH₂Cl₂ was treated with triphenyl phosphine (1.2 g, 4.59 mmol) at rt, and stirred for 1 h. The solvent was concentrated in vacuo, and the resulting residue was purified by silicagel column chromatography (eluent: CHCl₃) to give 950 mg of compound G5 as a colorless powder.

¹H NMR (CDCl₃) δ 8.37(1H, s), 8.22(2H, d, J=7.2 Hz), 7.67(1H, t, J=7.5 Hz), 7.53(2H, t, J=7.8 Hz), 7.44(1H, s), 7.14-6.97(3H, m), 4.38(2H, s), 4.33(2H, q, J=7.2 Hz), 4.25(2H, s), 3.66(3H, s), 1.19(3H, t, 7.2 Hz)

### Compound G6: 7-(4-Fluoro-2-piperidin-1-ylmethyl-benzyl)-4-hydroxy-1-methyl-2-oxo-1,2-dihydro-[1,5]naphthyridine-3-carboxylic acid ethyl ester

A mixture of compound G4 (200 mg, 0.36 mmol) and potassium carbonate (250 mg, 1.81 mmol) in EtOH (6 mL) was treated with piperidine (107 μL, 1.08 mmol) at rt. After the mixture was stirred at 60° C. for 1 h, it was concentrated in vacuo. To the obtained residue was added 10% citric acid, and then extracted three times with CHCl₃. The organic layer was dried over Na₂SO₄ and then concentrated under reduced pressure.

The obtained residue was triturated with ether to give 135 mg of compound G6 as a pale yellow powder.

¹H NMR (CDCl₃) δ 8.50(1H, br), 7.26(1H, bs), 7.12-6.90(3H, m), 4.52(2H, q, J=7.2 Hz), 4.30(2H, s), 3.52(3H, s), 3.30(2H, s), 2.29(3H, br), 1.70-1.30(7H, br), 1.48(3H, t, J=7.2 Hz)

### Compound G7: 7-[4-Fluoro-2-(2-oxo-pyrrolidin-1-ylmethyl)-benzyl]-4-hydroxy-1-methyl-2-oxo-1,2-dihydro-[1,5]naphthyridine-3-carboxylic acid ethyl ester

A mixture of NaH (60%, 160 mg, 4.01 mmol) in THF(15 mL) was treated with 2-pyrrolidone (381 μL, 5.01 mmol) at 0° C. After the mixture was stirred at 0° C. for 15 minutes, it was warmed to rt. The resulting mixture was treated with compound G5 (850 mg, 1.67 mmol) in THF (5 mL) at 0° C. and stirred for 30 min, and then at rt for 2 h. The reaction mixture was poured into ice/water, and it was acidified with 10% citric acid. The mixture was extracted three times with EtOAc, and the combined organic layer was washed with water and then brine. After dried over Na₂SO₄, the solvent was concentrated in vacuo to provide yellow oil. The obtained residue was purified by silicagel column chromatography (eluent: CHCl₃/EtOH=20/1) to give 321 mg of compound G7 as a colorless powder.

¹H NMR (CDCl₃) δ 8.36(1H, s), 7.60(1H, s), 7.24-6.94(3H, m), 4.52(2H, q, J=7.2 Hz), 4.39(2H, s), 4.19(2H, s), 3.62(3H, s), 3.17(2H, t, J=7.2 Hz), 2.29(2H, t, J=8.1 Hz), 1.90-1.80(2H, m), 1.48(3H, t, J=7.2 Hz)

### Compound G8: 7-(4-Fluoro-2-imidazol-1-ylmethyl-benzyl)-4-hydroxy-1-methyl-2-oxo-1,2-dihydro-[1,5]naphthyridine-3-carboxylic acid ethyl ester

A mixture of NaH (60%, 104 mg, 2.60 mmol) in THF (8 mL) was treated with imidazole (177 mg, 2.60 mmol) at 0° C. The mixture was stirred at 0° C. for 15 min, and the reaction temperature was raised to rt. To the resulting mixture was added compound G4 (600 mg, 1.08 mmol) in THF (4 mL) and DMF (0.5 mL) at 0° C., and it was stirred at 0° C. for 30 min then at rt for 2 h. After the reaction mixture was poured into ice/water, it was acidified with 10% citric acid. The product was extracted three times with CHCl₃, and the combined organic layer was washed with water and then brine. After dried over Na₂SO₄, the solvent was concentrated under reduced pressure. The obtained residue was triturated with isopropylether to give 430 mg of compound G8 as a pale brown powder.

¹H NMR (CDCl₃) δ 8.40(1H, s), 8.06(1H, s), 7.23-6.84(6H, m), 5.21(2H, s), 4.51(2H, q, J=7.2 Hz), 4.17(2H, s), 3.52(3H, s), 1.48(3H, t, J=7.2 Hz)

### EXAMPLE 27

### 7-(4-Fluoro-2-piperidin-1-ylmethyl-benzyl)-4-hydroxy-1-methyl-2-oxo-1,2-dihydro-[1,5]naphthyridine-3-carboxylic acid hydroxymethyl-amide

This compound was prepared in the similar manner as Example 1.

m.p.: 114-115° C.

ESI: m/z 469 (M+1)

¹H NMR (CDCl₃) δ 10.49(1H, br), 8.56(1H, d, J=1.8), 7.29(1H, br), 7.11-6.92(3H, m), 4.31(2H, s), 3.86(2H, t, J=4.8 Hz), 3.67-3.62(3H, m), 3.53(3H, s), 3.30(2H, s), 2.85(1H, br), 2.30(3H, br), 1.55-1.35(5H, m)

### EXAMPLE 28

### 7-(4-Fluoro-2-piperidin-1-ylmethyl-benzyl)-4-hydroxy-1-methyl-2-oxo-1,2-dihydro-[1,5]naphthyridine-3-carboxylic acid methoxymethyl-amide

This compound was prepared in the similar manner as Example 1.

m.p.: 139-140° C.

FAB-MS: m/z 483 (M+1)

¹H NMR (CDCl₃) δ 10.37(1H, br), 8.56(1H, d, J=1.8), 7.29(1H, br), 7.11-6.92(3H, m), 4.31(2H, s), 3.68-3.58(4H, m), 3.55(3H, s), 3.42(3H, s), 3.30(2H, s), 2.29(3H, br), 1.60-1.35(7H, m)

### EXAMPLE 29

### 7-[4-Fluoro-2-(2-oxo-pyrrolidin-1-ylmethyl)-benzyl]-4-hydroxy-1-methyl-2-oxo-1,2-dihydro-[1,5]naphthyridine-3-carboxylic acid hydroxymethyl-amide

This compound was prepared in the similar manner as Example 1.

m.p.: 162-163° C.

FAB-MS: m/z 469 (M+1)

¹H NMR (CDCl₃) δ 10.49(1H, br), 8.38(1H, s), 7.64(1H, s), 7.25-6.94(3H, m), 4.39(2H, s), 4.19(2H, s), 3.86(2H, t, J=4.8 Hz), 3.66-3.50(5H, m), 3.17(2H, t, J=7.2 Hz), 2.28(2H, t, J=8.1 Hz), 1.91-1.80(2H, m)

### EXAMPLE 30

### 7-[4-Fluoro-2-(2-oxo-pyrrolidin-1-ylmethyl)-benzyl]-4-hydroxy-1-methyl-2-oxo-1,2-dihydro-[1,5]naphthyridine-3-carboxylic acid methoxymethyl-amide

This compound was prepared in the similar manner as Example 1.

m.p.: 150-151° C.

FAB-MS: m/z 483 (M+1)

¹H NMR (CDCl₃) δ 10.37(1H, br), 8.40(1H, s), 7.65(1H, s), 7.25-6.94(3H, m), 4.39(2H, s), 4.19(2H, s), 3.70-3.60(7H, m), 3.42(3H, s), 3.16(2H, t, J=7.5 Hz), 2.28(2H, t, J=8.4 Hz), 1.90-1.82(2H, m)

### EXAMPLE 31

### 7-(4-Fluoro-2-imidazol-1-ylmethyl-benzyl)-4-hydroxy-1-methyl-2-oxo-1,2-dihydro-[1,5]naphthyridine-3-carboxylic acid hydroxymethyl-amide

This compound was prepared in the similar manner as Example 1.

m.p.: >335° C.

ESI: m/z 452 (M+1)

¹H NMR (CDCl₃) δ 10.40(1H, br), 8.38(1H, s), 7.66(1H, br), 7.21-7.06(4H, m), 6.85-6.82(2H, m), 5.15(2H, s), 4.14(2H, s), 3.88(2H, t, J=4.8 Hz), 3.66-3.61(2H, m), 3.50(3H,s)

### EXAMPLE 32

### 7-(4-Fluoro-2-imidazol-1-ylmethyl-benzyl)-4-hydroxy-1-methyl-2-oxo-1,2-dihydro-[1,5]naphthyridine-3-carboxylic acid methoxymethyl-amide

This compound was prepared in the similar manner as Example 1.

m.p.: 184-185° C.

ESI: m/z 466 (M+1)

1H NMR (CDCl₃) δ 10.31(1H, br), 8.46(1H, s), 8.00(1H, br), 7.23-7.06(4H, m), 6.89-6.85(2H, m), 5.21(2H, s), 4.18(2H, s), 3.67-3.58(4H, m), 3.53(3H, s), 3.42(3H,s)

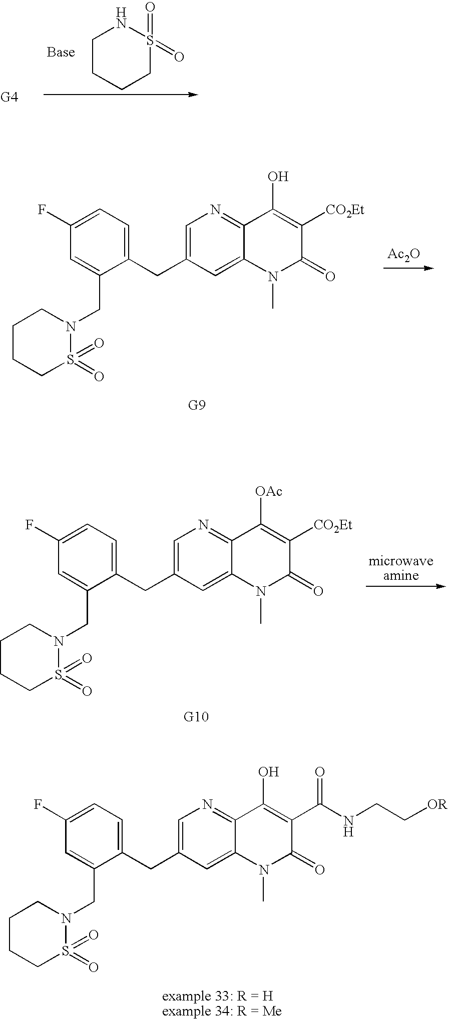

### Compound G9: 7-[2-(1,1-Dioxo-1lambda*6*-[1,2]thiazinan-2-ylmethyl)-4-fluoro-benzyl]-4-hydroxy-1-methyl-2-oxo-1,2-dihydro-[1,5]naphthyridine-3-carboxylic acid ethyl ester

### Compound G10: 4-Acetoxy-7-[2-(1,1-dioxo-1lambda*6*-[1,2]thiazinan-2-ylmethyl)-4-fluoro-benzyl]-1-methyl-2-oxo-1,2-dihydro-[1,5]naphthyridine-3-carboxylic acid ethyl ester

To a suspension of NaH (60% in oil, 60 mg, 1.5 mmol) in THF (3 mL) was added [1,2]thiazinane 1,1-dioxide (243 mg, 1.8 mmol) under ice cooling and the mixture was stirred for 15 min. After the resulting mixture was stirred additionally at rt for 10 min, the mixture was cooled again. To the mixture was added dropwise a solution of compound G4 (333 mg, 0.6 mmol) in THF (6 mL) and stirred at rt for 1 h and then heated at 50° C. for 15 min with stirring. The reaction mixture was treated with 1N HCl (15 mL) and extracted with EtOAc. The extract was washed with brine and dried over Na₂SO₄. The solvent was concentrated in vacuo to afford the crude product, and the product mixture was purified by silicagel column chromatography (eluent: CHCl₃/MeOH=10/1 v/v) to give 300 mg of compound G9.

The mixture of the crude product of compounds G9 (300 mg) and acetic anhydride (5 mL) was heated at 120° C. for 2 hr with stirring. The solvent was removed in vacuo to afford the crude product, and the crude product mixture was purified by silicagel column chromatography (eluent: n-Hexane/EtOAc=1/1 v/v) to afford 182 mg of compound G10 (yield=61.0% from compound G4).

¹H NMR (CDCl₃) δ 8.29(1H, d, J=1.8 Hz), 7.62(1H, d, J=1.5 Hz), 7.26-7.24(1H, m), 7.10-7.07(1H, m), 7.06-6.98(1H, m), 4.42(2H, q, J=6.9 Hz), 4.26(2H, s), 4.17(2H, s), 3.66(3H, s), 3.08(2H, t, J=5.1 Hz), 2.90(2H, t, J=6.0 Hz), 2.40(3H, s), 2.20-2.08(2H, m), 1.55-1.40(2H, m), 1.38(3H, t, J=7.2 Hz).

### EXAMPLE 33

### 7-[2-(1,1-Dioxo-1lambda*6*-[1,2]thiazinan-2-ylmethyl)-4-fluoro-benzyl]-4-hydroxy-1-methyl-2-oxo-1,2-dihydro-[1,5]naphthyridine-3-carboxylic acid(2-hydroxy-ethyl)-amide

This compound was prepared from compound G10 in a manner similar to that described in Example 1:

m.p.: 182-183° C.

Elemental Analysis: C₂₄H₂₇F₁N₄O₆

Calcd (%): C, 55.59; H, 5.25; N, 10.80.

Found (%): C, 55.28; H, 5.28; N, 10.61.

¹H NMR (DMSO-d6) δ 10.43(1H, bs), 8.40(1H, s), 7.81(1H, s), 7.32-7.13(3H, m), 4.33(2H, s), 4.24(2H, s), 3.53-3.50(5H, m), 3.42-3.33(2H, m), 3.20-3.10(4H, m), 2.59(1H, t, J=5.4 Hz), 2.02(2H, bs), 1.56(2H, bs).

### EXAMPLE 34

### 7-[2-(1,1-Dioxo-1lambda*6*-[1,2]thiazinan-2-ylmethyl)-4-fluoro-benzyl]-4-hydroxy-1-methyl-2-oxo-1,2-dihydro-[1,5]naphthyridine-3-carboxylic acid(2-methoxy-ethyl)-amide

This compound was prepared from compound G10 in a manner similar to that described in Example 1:

m.p.: 123-125° C.

¹H NMR (DMSO-d6) δ 10.41(1H, bs), 8.46(1H, s), 7.89(1H, s), 7.30-7.13(3H, m), 4.33(2H, s), 4.27(2H, s), 3.57-3.53(7H, m), 3.30(3H, s), 3.19-3.14(4H, m), 2.03(2H, bs), 1.57(2H, bs).

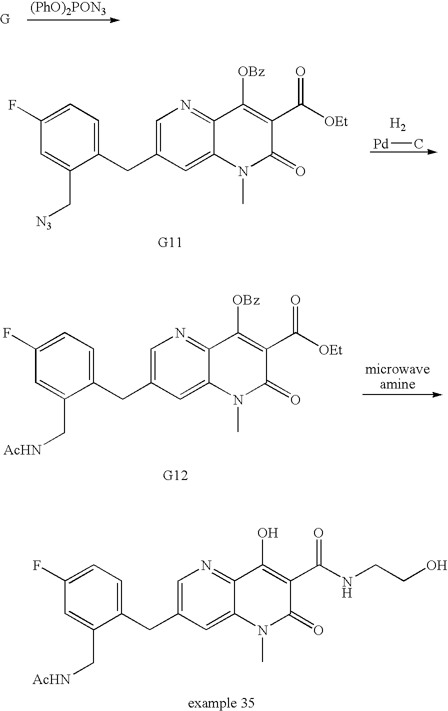

### Compound G11: 7-(2-Azidomethyl-4-fluoro-benzyl)-4-benzoyloxy-1-methyl-2-oxo-1,2-dihydro-[1,5]naphthyridine-3-carboxylic acid ethyl ester

To a solution of compound G (366 mg, 0.746 mmol) in THF (19 mL) were added P(Ph)₃ (292 mg, 1.11 mmol) at rt and azodicarboxylic acid diisopropyl in toluene (0.563 ml, 40 wt %) and diphenyl(phosphoryl)azide (0.240 ml, 1.11 mmol) with ice-cooling, and then the mixture was stirred at rt for 30 min. The solvent was concentrated in vacuo to afford the crude product, the crude product was purified by silicagel column chromatography (eluent: n-Hexane/EtOAc=1/1 v/v) to give 317 mg of compound G11 (yield=82.4%) as a colorless crystal.

¹H NMR (CDCl₃) δ 8.34(1H, d, J=1.8 Hz), 8.20-8.23(2H, m), 7.65-7.69(1H, m), 7.50-7.55(2H, m), 7.39(1H, d, J=1.8 Hz), 7.05-7.15 (3H, m), 4.32(2H, q, J=7.2 Hz), 4.28(2H, s), 4.18(2H, s), 3.65(3H, s), 1.19(3H, t, J=7.2 Hz).

### Compound G12: 7-[2-(Acetylamino-methyl)-4-fluoro-benzyl]-4-benzoyloxy-1-methyl-2-oxo-1,2-dihydro-[1,5]naphthyridine-3-carboxylic acid ethyl ester

A mixture of compound G11 (177 mg, 0.343 mmol) in 1,4-dioxane (3 mL), 10% Pd-C (18 mg) and acetic anhydride (0.162 mL) was stirred at rt for 1.5 hr under H₂ atmosphere. After the insololuble materials were filtered off, the filtrate was concentrated in vacuo to afford the crude product. The crude product was purified by silicagel column chromatography (eluent: CHCl₃/MeOH=30/1 v/v) to give 96 mg of compound G12 (yield=53.0%) as a colorless crystal.

¹H NMR (CDCl₃) δ 8.23(1H, s), 8.18-8.21(2H, m), 7.64-7.69(1H, m), 7.49-7.56(3H, m), 7.13-7.18 (1H, m), 6.95-7.01 (2H, m), 5.63(1H, bs), 4.29-4.37(4H, m), 4.16(2H, s), 3.70(3H, s), 1.92(3H, s), 1.18(3H, t, J=7.2 Hz).

### EXAMPLE 35

### 7-[2-(Acetylamino-methyl)-4-fluoro-benzyl]-4-hydroxy-1-methyl-2-oxo-1,2-dihydro-[1,5]naphthyridine-3-carboxylic acid(2-hydroxy-ethyl)-amide

This compound was prepared from compound G12 in a manner similar to that described in Example 1 and was obtained at 97.0% yield:

m.p.: 202-205° C.

Elemental Analysis: C₂₂H₂₃F₁N₄O₅(H₂O)_{1.2}

Calcd (%): C, 56.94; H, 5.52; N, 12.07; F, 4.09.

Found (%): C, 56.54; H, 5.81; N, 13.63; F, 3.62.

¹H NMR (DMSO-d6) δ 10.43(1H, t, J=5.7 Hz), 8.38(1H, s), 8.33(1H, t, J=5.7 Hz), 7.78(1H, s), 7.22-7.28(1H, m), 7.04-7.09(2H, m), 4.28(2H, t, J=5.7 Hz), 4.19(2H, s), 3.55(2H, t, J=5.7 Hz), 3.53(3H, s), 2.60(2H, t, J=5.7 Hz), 1.83(3H, s).

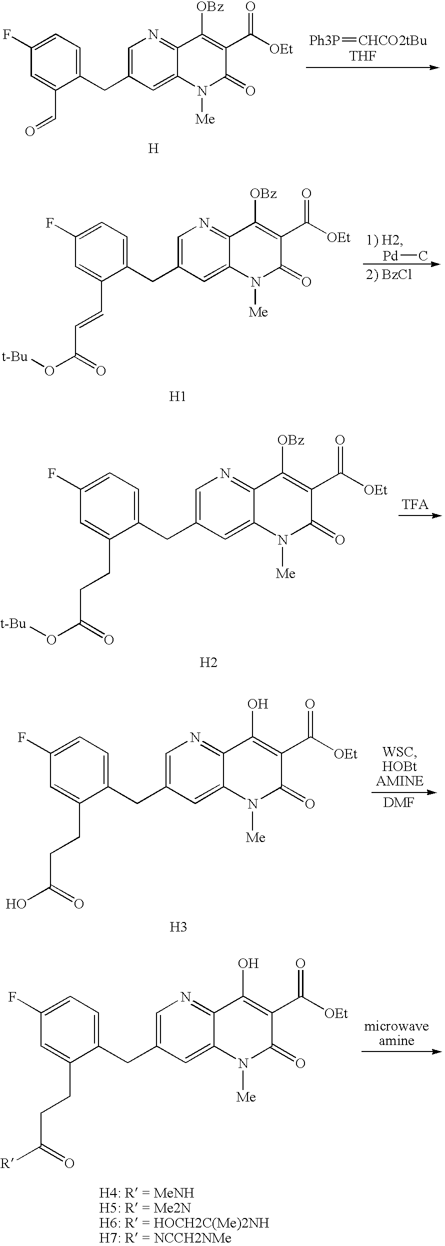

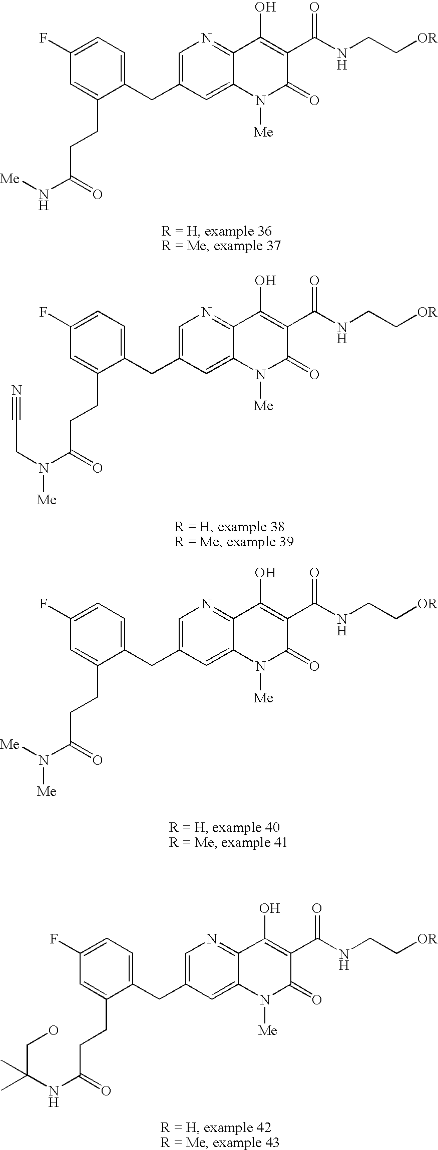

### Compound H1: 4-Benzoyloxy-7-[2-((E)-2-tert-butoxycarbonyl-vinyl)-4-fluoro-benzyl]-1-methyl-2-oxo-1,2-dihydro-[1,5]naphthyridine-3-carboxylic acid ethyl ester

A mixture of compound H (1.65 g) in THF (33 mL) and (t-butoxycarbonymethylene)triphenylphosphoran (1.65 g) was stirred at rt overnight. After removal of the solvent in vacuo, the residue was purified by silicagel column chromatography (eluent: n-Hexane/EtOAc) to give 1.88 g of compound H1 (yield=94.6%) as a colorless solid.

¹H NMR (CDCl₃) δ: 8.30(1H, d, J=1.8 Hz), 8.18-8.22(2H, m), 7.81(1H, dd, J=1.5 Hz, 15.6 Hz), 7.66(1H, m), 7.48-7.55(3H, m), 7.18-7.29(2H, m), 7.04(1H, m), 6.23(1H, d, J=15.6 Hz), 4.32(2H, q, J=7.2 Hz), 4.22(2H, s), 3.68(3H, s), 1.50(9H, s), 1.18(3H, t, J=7.2 Hz).

### Compound H2: 4-Benzoyloxy-7-[2-(2-tert-butoxycarbonyl-ethyl)-4-fluoro-benzyl]-1-methyl-2-oxo-1,2-dihydro-[1,5]naphthyridine-3-carboxylic acid ethyl ester

A mixture of compound H1 (2.11 g) in EtOAc (50 mL) and 10% Pd-C (200 mg) was stirred at rt for 4 h under H₂ atmosphere, and the mixture was filtered though a pad of Celite. The filtrate was concentrated in vacuo, the residue was dissolved in EtOAc. A mixture of the solution and 10% Pd-C (200 mg) was stirred at rt overnight under H₂ atmosphere, and the mixture was filtered though a pad of Celite. The filtrate was concentrated in vacuo to afford the mixture of compound H2 and de-benzoyl compound H2 (5:1) as yellow amorphous.

To a mixture of above-mentioned mixture and Et₃N (150 μL) in CH₂Cl₂ (10 mL) was added benzoyl chloride (88.5 μL) at 0° C., and the mixture was stirred at rt for 1.5 h. The reaction mixture was quenched with NH₄Cl solution, and extracted three times with EtOAc. The EtOAc layer was washed with water and brine, dried over Na₂SO₄, and then the solvent was concentrated in vacuo. The resulting amorphous purified by silicagel column chromatography to afford 1.95 g of compound H2 as colorless amorphous.

¹H NMR (CDCl₃) δ: 8.33(1H, d, J=1.8 Hz), 8.19-8.23(2H, m), 7.67(1H, m), 7.50-7.55(2H, m), 7.41(1H, m), 7.06(1H, dd, J=5.7 Hz, 8.1 Hz), 6.86-6.95(2H, m), 4.33(2H, q, J=7.2 Hz), 4.15(2H, s), 3.66(3H, s), 2.84(2H, t, J=7.5 Hz), 2.45(2H, t, J=7.5 Hz), 1.40(9H, s), 1.18(3H, t, J=7.2 Hz).

### Compound H3: 7-[2-(2-Carboxy-ethyl)-4-fluoro-benzyl]-4-hydroxy-1-methyl-2-oxo-1,2-dihydro-[1,5]naphthyridine-3-carboxylic acid ethyl ester

A mixture of compound H2 (1.65 g) and trifluoroacetic acid (5 mL) was stirred at rt overnight. After the solvent was removed in vacuo, MeOH and NH₄OH solution were added to the residue. The resulting precipitate was collected by filtration washing with MeOH to give 917 mg of compound H3 (yield=76.0%) as a colorless solid.

¹H NMR (DMSO-d6) δ: 12.2(1H, br), 8.37(1H, s), 7.83(1H, s), 7.22(1H, m), 6.97-7.11(2H, m), 4.24(2H, q, J=7.2 Hz), 4.24(2H, s), 3.50(3H, s), 2.87(2H, t, J=7.8 Hz), 2.42(2H, t, J=7.8 Hz), 1.26(3H, t, J=7.2 Hz).

### Compound H4: 7-[4-Fluoro-2-(2-methylcarbamoyl-ethyl)-benzyl]-4-hydroxy-1-methyl-2-oxo-1,2-dihydro-[1,5]naphthyridine-3-carboxylic acid ethyl ester

This compound was prepared from compound H3 in a manner similar to that described in compound D6:

¹H NMR (CDCl₃) δ: 7.43(1H, d, J=1.8 Hz), 7.38(1H, m), 7.10(1H, m), 6.88-6.96(2H, m), 5.31(1H, br), 4.52(2H, q, J=7.2 Hz), 4.21(2H, s), 3.57(3H, s), 2.94(2H, t, J=7.8 Hz), 2.77(3H, d, J=4.5 Hz), 2.35(2H, t, J=7.8 Hz), 1.48(3H, t, J=7.2 Hz).

### Compound H5: 7-[2-(2-Dimethylcarbamoyl-ethyl)-4-fluoro-benzyl]-4-hydroxy-1-methyl-2-oxo-1,2-dihydro-[1,5]naphthyridine-3-carboxylic acid ethyl ester

This compound was prepared from compound H3 in a manner similar to that described in compound D6:

¹H NMR (CDCl₃) δ: 13.8(1H, br), 8.45(1H, d, J=1.8 Hz), 7.38(1H, d, J=1.8 Hz), 7.11 (1H, dd, J=6.0 Hz, 8.1 Hz), 6.88-6.95(2H,m), 4.51(2H, q, J=7.2 Hz), 4.22(2H, s), 3.56(3H, s), 2.92(2H, t, J=7.5 Hz), 2.91(3H, s), 2.89(3H, s), 2.47(2H, t, J=7.5 Hz), 1.48(3H, t, J=7.2 Hz).

### Compound H6: 7-{4-Fluoro-2-[2-(2-hydroxy-1,1-dimethyl-ethylcarbamoyl)-ethyl]-benzyl}-4-hydroxy-1-methyl-2-oxo-1,2-dihydro-[1,5]naphthyridine-3-carboxylic acid ethyl ester

This compound was prepared from compound H3 in a manner similar to that described in compound D6:

¹H NMR (CDCl₃) δ: 8.41(1H, d, J=1.8 Hz), 7.39(1H, d, J=1.8 Hz), 7.09(1H, m), 5.5(1H, br), 6.91-6.95(2H, m), 4.52(2H, q, J=7.2 Hz), 4.20(2H, s), 3.56(3H, s), 3.53(2H, s), 2.91(2H, t, J=7.5 Hz), 2.34(2H, t, J=7.5 Hz), 1.48(3H, t, J=7.2 Hz), 1.23(6H, s).

### Compound H7: 7-{2-[2-(Cyanomethyl-methyl-carbamoyl)-ethyl]-4-fluoro-benzyl}-4-hydroxy-1-methyl-2-oxo-1,2-dihydro-[1,5]naphthyridine-3-carboxylic acid ethyl ester

This compound was prepared from compound H3 in a manner similar to that described in compound D6:

¹H NMR (CDCl₃) δ: 8.44(1H, d, J=1.5 Hz), 7.36(1H, m), 7.11(1H, m), 6.91-6.96(2H, m), 4.52(2H, q, J=7.2 Hz), 4.32(s), 4.22(2H, s), 3.56(3H, s), 3.05(s), 2.96(s), 2.94(2H, t, J=7.5 Hz), 2.89(s), 2.56(2H, t, J=7.5 Hz), 1.48(3H, t, J=7.2 Hz).

### EXAMPLE 36

### 7-[4-Fluoro-2-(2-methylcarbamoyl-ethyl)-benzyl]-4-hydroxy-1-methyl-2-oxo-1,2-dihydro-[1,5]naphthyridine-3-carboxylic acid(2-hydroxy-ethyl)-amide

This compound was prepared from compound H4 in a manner similar to that described in Example 1:

HRMS(FAB): C₂₃H₂₅FN₄O₅+H. Calcd: 457.1887. Found: 457.1878 (Int 100%).

¹H NMR (CDCl₃) δ 10.49(1H, brt, J=5.7 Hz), 8.49(1H, d, J=1.5 Hz), 7.44(1H, m), 7.10(1H, m), 6.95-6.88(2H, m), 5.32(1H, br), 4.22(2H, s), 3.87(2H, dt, J=5.1 Hz, 4.8 Hz), 3.65(2H, dt, J=5.7 Hz, 4.8 Hz), 3.60(3H, s), 2.94(2H, t, J=7.8 Hz), 2.76(3H, d, J=5.1 Hz), 2.34(2H, t, J=7.8 Hz), 2.24(1H, brt, J=5.1 Hz).

### EXAMPLE 37

### 7-[4-Fluoro-2-(2-methylcarbamoyl-ethyl)-benzyl]-4-hydroxy-1-methyl-2-oxo-1,2-dihydro-[1,5]naphthyridine-3-carboxylic acid(2-methoxy-ethyl)-amide

This compound was prepared from compound H4 in a manner similar to that described in Example 1:

HRMS(FAB): C₂₄H₂₈FN₄O₅+H. Calcd: 471.2044. Found: 471.2030 (Int 100%).

¹H NMR (CDCl₃) δ 10.37(1H, brt), 8.50(1H, d, J=1.8 Hz), 7.43(1H, d, J=1.8 Hz), 7.10(1H, m), 6.95-6.87(2H, m), 4.21(2H, s), 3.90-3.64(2H, m), 3.60(3H, s), 3.61-3.56(2H, m), 2.94(2H, t, J=7.5 Hz), 2.77(3H, d, J=5.1 Hz), 2.33(2H, t, J=7.5 Hz).

### EXAMPLE 38

### 7-{2-[2-(Cyanomethyl-methyl-carbamoyl)-ethyl]-4-fluoro-benzyl}-4-hydroxy-1-methyl-2-oxo-1,2-dihydro-[1,5]naphthyridine-3-carboxylic acid(2-hydroxy-ethyl)-amide

This compound was prepared from compound H7 in a manner similar to that described in Example 1:

HRMS(FAB): C₂₅H₂₇FN₅O₅+H. Calcd: 496.1996. Found: 496.2012 (Int 100%).

¹H NMR (CDCl₃) δ 10.48(1H, brt, J=5.7 Hz)), 8.49(1H, d, J=1.5 Hz), 7.43(1H, m), 7.13(1H, m), 6.97-6.90(2H, m), 4.30(2H, s), 4.22(2H, s), 3.86(2H, dt, J=5.4 Hz, 4.8 Hz), 3.65(2H, dt, J=5.7 Hz, 4.8 Hz), 3.60(3H, s), 3.03(3H, s), 2.94(2H, t, J=7.5 Hz), 2.51(2H, t, J=7.5 Hz), 2.27(2H, t, J=7.5 Hz).

### EXAMPLE 39

### 7-{2-[2-(Cyanomethyl-methyl-carbamoyl)-ethyl]-4-fluoro-benzyl}-4-hydroxy-1-methyl-2-oxo-1,2-dihydro-[1,5]naphthyridine-3-carboxylic acid(2-methoxy-ethyl)-amide

This compound was prepared from compound H7 in a manner similar to that described in Example 1:

HRMS(FAB): C₂₅H₂₇FN₅O₅+H. Calcd: 496.1996. Found: 496.2012 (Int 100%).

¹H NMR (CDCl₃) δ 10.48(1H, brt, J=5.7 Hz), 8.49(1H, d, J=1.5 Hz), 7.43(1H, m), 7.13(1H, m), 6.97-6.90(2H, m), 4.30(2H, s), 4.22(2H, s), 3.86(2H, dt, J=5.4 Hz, 4.8 Hz), 3.65(2H, dt, J=5.7 Hz, 4.8 Hz), 3.60(3H, s), 3.03(3H, s), 2.94(2H, t, J=7.5 Hz), 2.51(2H, t, J=7.5 Hz), 2.27(2H, t, J=7.5 Hz).

### EXAMPLE 40

### 7-[2-(2-Dimethylcarbamoyl-ethyl)-4-fluoro-benzyl]-4-hydroxy-1-methyl-2-oxo-1,2-dihydro-[1,5]naphthyridine-3-carboxylic acid(2-hydroxy-ethyl)-amide

This compound was prepared from compound H5 in a manner similar to that described in Example 1:

HRMS(FAB): C₂₄H₂₈FN₄O₅+H. Calcd: 471.2044. Found: 471.2036 (Int 100%).

¹H NMR (CDCl₃) ε 10.51(1H, brt), 8.51(1H, d, J=1.8 Hz), 7.45(1H, d, J=1.8 Hz), 7.12(1H, dd, J=6.3 Hz, 7.2 Hz), 6.96-6.87(2H, m), 4.23(2H, s), 3.86(2H, dt, J=5.4 Hz, 4.8 Hz), 3.65(2H, dt, J=5.7 Hz, 4.8 Hz), 3.60(3H, s), 2.93(2H, t, J=7.8 Hz), 2.91(3H, s), 2.88(3H, s), 2.46(2H, t, J=7.8 Hz), 2.20(2H, brt).

### EXAMPLE 41

### 7-[2-(2-Dimethylcarbamoyl-ethyl)-4-fluoro-benzyl]-4-hydroxy-1-methyl-2-oxo-1,2-dihydro-[1,5]naphthyridine-3-carboxylic acid(2-methoxy-ethyl)-amide

This compound was prepared from compound H5 in a manner similar to that described in Example 1:

HRMS(FAB): C₂₅H₃₀FN₄O₅+H. Calcd: 485.2200. Found: 485.2215 (Int 100%).

¹H NMR (CDCl₃) δ 10.37(1H, brt), 8.50(1H, d, J=1.8 Hz), 7.44(1H, d, J=1.8 Hz), 7.11(1H, dd, J=6.0 Hz, 8.7 Hz), 6.96-6.87(2H, m), 4.23(2H, s), 3.69-3.63(2H, m), 3.60(3H, s), 3.61-3.57(2H, m), 2.93(2H, t, J=7.8 Hz), 2.90(3H, s), 2.88(3H, s), 2.45(2H, t, J=7.8 Hz).

### EXAMPLE 42

### 7-{4-Fluoro-2-[2-(2-hydroxy-1,1-dimethyl-ethylcarbamoyl)-ethyl]-benzyl}-4-hydroxy-1-methyl-2-oxo-1,2-dihydro-[1,5]naphthyridine-3-carboxylic acid(2-hydroxy-ethyl)-amide

This compound was prepared from compound H6 in a manner similar to that described in Example 1:

HRMS(FAB): C₂₆H₃₂FN₄O₅+H. Calcd: 515.2306. Found: 515.2292 (Int 43.2%).

¹H NMR (DMSO-d6) δ 10.4(brt), 8.4(d), 7.9(d), 7.61-7.57(m), 7.44-7.39(m), 7.10-7.01(m), 5(br), 4.2(s), 3.53(m), 2.80(m), 2.3(t), 1.13(s).

### EXAMPLE 43

### 7-{4-Fluoro-2-[2-(2-hydroxy-1,1-dimethyl-ethylcarbamoyl)-ethyl]-benzyl}-4-hydroxy-1-methyl-2-oxo-1,2-dihydro-[1,5]naphthyridine-3-carboxylic acid(2-methoxy-ethyl)-amide

This compound was prepared from compound H6 in a manner similar to that described in Example 1:

HRMS(FAB): C₂₇H₃₄FN₄O₅+H. Calcd: 529.2462. Found: 529.2447 (Int 100%).

¹H NMR (CDCl₃) δ 10.36(1H, brt), 8.49(1H, d, J=1.8 Hz), 7.42(1H, d, J=1.8 Hz), 7.14-7.08(1H, m), 6.95-6.88(2H, m), 5.35(1H, brs), 4.32(1H, brt, J=5.4 Hz), 4.21(2H, s), 3.69-3.63(2H, m), 3.60(3H, s), 3.61-3.57(2H, m), 3.52(2H, brd, J=5.4 Hz), 3.42(3H, s), 2.91(2H, t, J=7.5 Hz), 2.30(2H, t, J=7.5 Hz), 1.54(6H, s).

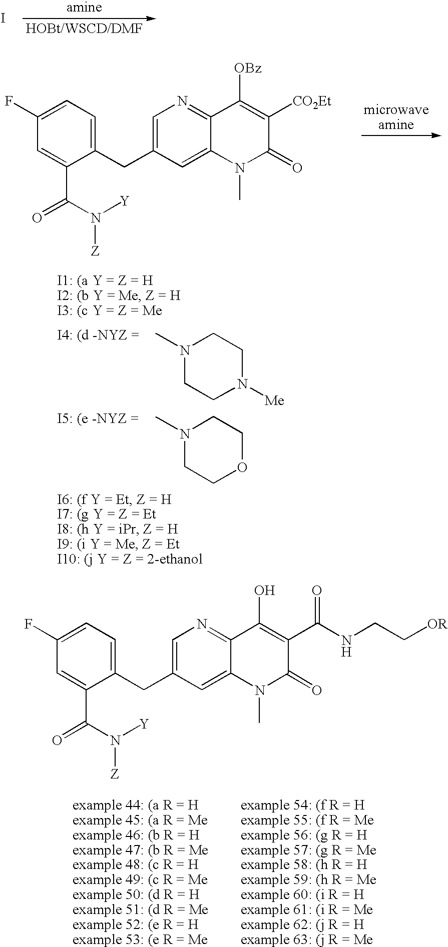

### Compound I1: 7-(2-Carbamoyl-4-fluoro-benzyl)-4-benzoyloxy-1-methyl-2-oxo-1,2-dihydro-[1,5]naphthyridine-3-carboxylic acid ethyl ester

To a solution of compound I (467.7 mg, 0.927 mmol), HOBt (150.3 mg, 1.11 mmol), WSCD (266.6 mg, 1.39 mmol) in DMF (10 mL) were added NH₄Cl solution (198.4 mg. 3.71 mmol) and ⁱPr₂NEt at rt, and stirred for 5 h. After the mixture was treated with aqueous 10% citric acid, the mixture was washed with EtOAc and diethyl ether. The resulting precipitate in an aqueous layer was filtered and washed with EtOAc and Et₂O to give 260 mg of compound I1 (yield=70.0%) as a colorless crystal.

¹H NMR (DMSO-d6) δ 8.47(1H, s), 7.97 (1H, s), 7.88(1H, s), 7.60(1H, s), 7.46-7.42(1H, m), 7.26(2H, d, J=8.4 Hz), 4.34(2H, s), 4.23(2H, q, J=7.2 Hz), 3.50(3H,s), 1.26 (3H, 7.2 Hz).

### Compound I2: 4-Benzoyloxy-7-(4-fluoro-2-methylcarbamoyl-benzyl)-1-methyl-2-oxo-1,2-dihydro-[1,5]naphthyridine-3-carboxylic acid ethyl ester

This compound was prepared from compound I in a manner similar to that described in compound I1 and was obtained at 89.0% yield:

¹H NMR (CDCl₃) δ 8.35(1H, s), 8.21(2H, d, J=8.4 Hz), 7.77(1H, s), 7.67(1H, t, J=7.5 Hz), 7.52(2H, t, J=7.2 Hz), 7.25-7.20(1H, m), 7.15-7.02(2H, m), 5.70(1H, bs), 4.32-4.28(4H, m), 3.69(3H, s), 2.88(3H, d, J=4.8 Hz), 1.17(3H, t, J=7.2 Hz).

### Compound I3: 4-Benzoyloxy-7-(2-dimethylcarbamoyl-4-fluoro-benzyl)-1-methyl-2-oxo-1,2-dihydro-[1,5]naphthyridine-3-carboxylic acid ethyl ester

This compound was prepared from compound I in a manner similar to that described in compound I1 and was obtained at 92.0% yield:

(CDCl₃) δ: 1.19(3H, t, J=7.2 Hz), 2.54(3H, s), 2.95(2H, s), 3.69(3H, s), 4.09(2H, brs), 4.33(2H, q, J=7.2 Hz), 6.93(1H, dd, J=2.7 Hz, 8.4 Hz), 7.04(1H, ddd, J=2.8 Hz, 8.4 Hz, 8.4 Hz), 7.23(1H, dd, J=5.4 Hz, 8.4 Hz), 7.49-7.55(2H, m), 7.63-7.70(2H, m), 8.18-8.22(2H, m), 8.33(1H, d, J=1.8 Hz).

### Compound I4: 4-Benzoyloxy-7-[4-fluoro-2-(4-methyl-piperazine-1-carbonyl)-benzyl]-1-methyl-2-oxo-1,2-dihydro-[1,5]naphthyridine-3-carboxylic acid ethyl ester

This compound was prepared from compound I in a manner similar to that described in compound I1 and was obtained at 59.0% yield:

¹H NMR (CDCl₃) δ 8.34 (d, J=1.8 Hz, 1H), 8.22 (s, 1H), 8.19 (d, 1.8 Hz, 1H), 7.69 (s, 1H), 7.68 (m, 1H), 7.52 (t, J=8.1 Hz, 2H), 7.21 (m, 1H), 7.05 (m, 1H), 7.13 (m, 1H), 4.32 (q, J=4.2 Hz, 2H), 4.30-3.90 (m, 2H), 3.76 (s, 2H), 3.69 (s, 3H), 3.17 (s, 1H), 3.05 (s, 1H), 2.45 (s, 1H), 2.27 (s, 3H), 1.96 (s, 1H), 1.18 (t, J=6.90 Hz, 3H).

### Compound I5: 4-Benzoyloxy-7-[4-fluoro-2-(morpholine-4-carbonyl)-benzyl]-1-methyl-2-oxo-1,2-dihydro-[1,5]naphthyridine-3-carboxylic acid ethyl ester

This compound was prepared from compound I in a manner similar to that described in compound I1 and was obtained at 92.0% yield:

¹H NMR (CDCl₃) δ 8.35(1H, s), 8.21(2H, d, J=8.4 Hz), 7.69-7.64(2H, m), 7.53(2H, t, J=7.1 Hz), 7.24-7.19(1H, m), 7.09-7.02(1H, m), 6.93(1H, dd, J=2.7, 8.1 Hz), 4.33(2H, q, 7.2 Hz), 4.20-2.80(10H, m, br), 3.69(3H, s), 1.19(3H, t, 7.2 Hz),

### Compound I6: 4-Benzoyloxy-7-(2-ethylcarbamoyl-4-fluoro-benzyl)-1-methyl-2-oxo-1,2-dihydro-[1,5]naphthyridine-3-carboxylic acid ethyl ester

This compound was prepared from compound I in a manner similar to that described in compound I1 and was obtained at 85.0% yield:

¹H NMR (CDCl₃) δ 8.34(1H, s), 8.21-8.18(2H, m), 7.77(1H, s), 7.68-7.63(1H, m), 7.54-7.49(2H, m), 7.25-7.19(1H, m), 7.11-7.04(2H, m), 5.70(1H, bs), 4.35-4.28(4H, m), 3.69(3H, s), 3.40-3.31(2H, m), 1.23-1.11(6H, m).

### Compound I7: 4-Benzoyloxy-7-(2-diethylcarbamoyl-4-fluoro-benzyl)-1-methyl-2-oxo-1,2-dihydro-[1,5]naphthyridine-3-carboxylic acid ethyl ester

This compound was prepared from compound I in a manner similar to that described in compound I1 and was obtained at 91.0% yield:

¹H NMR (CDCl₃) δ 8.37(1H, s), 8.22-8.19(2H, m), 7.70-7.64(2H, m), 7.54-7.49(2H, m), 7.19-7.14(1H, m), 7.04-6.92(2H, m), 4.32(2H, q, 7.2 Hz), 4.05(2H, bs), 3.68(3H, s), 3.58-3.40(2H, m), 3.00-2.85(2H, m), 1.20-1.15(6H, m), 1.04-1.00(3H, t, J=7.2 Hz).

### Compound I8: 4-Benzoyloxy-7-(4-fluoro-2-isopropylcarbamoyl-benzyl)-1-methyl-2-oxo-1,2-dihydro-[1,5]naphthyridine-3-carboxylic acid ethyl ester

This compound was prepared from compound I in a manner similar to that described in compound I1 and was obtained at 70.0% yield:

¹H NMR (CDCl₃) δ 8.34(1H, s), 8.21-8.18(2H, m), 7.73(1H,s), 7.69-7.64(1H, m), 7.54-7.49(2H, m), 7.23-7.18(1H, m), 7.09-7.04(2H, m), 5.54(1H, bs), 4.35-4.28(4H, m), 4.16-4.10(1H, m), 3.69(3H, s), 1.23-1.13(6H, m).

### Compound I9: 4-Benzoyloxy-7-[2-(ethyl-methyl-carbamoyl)-4-fluoro-benzyl]-1-methyl-2-oxo-1,2-dihydro-[1,5]naphthyridine-3-carboxylic acid ethyl ester

This compound was prepared from compound I in a manner similar to that described in compound I1 and was obtained at 62.0% yield:

¹H NMR (CDCl₃) δ 8.36(1H, s), 8.21-8.19(2H, m), 7.70-7.66(1H, m), 7.54-7.50(2H, m), 7.23-7.19(1H, m), 7.09-6.90(2H, m), 4.32(2H, q, J=7.2 Hz), 4.08(2H, bs), 3.69(3H, s), 3.50-2.58(5H, m), 1.23-1.12(6H,m).

### Compound I10: 4-Benzoyloxy-7-{2-[bis-(2-hydroxy-ethyl)-carbamoyl]-4-fluoro-benzyl}-1-methyl-2-oxo-1,2-dihydro-[1,5]naphthyridine-3-carboxylic acid ethyl ester

This compound was prepared from compound I in a manner similar to that described in compound I1 and was obtained at 44.0% yield:

¹H NMR (CDCl₃) δ 8.36 (d, J=1.8 Hz, 1H), 8.21-8.18 (m, 2H), 7.70-7.65 (m, 2H), 7.53 (t, J=7.5 Hz, 2H), 7.22-7.18 (m, 1H), 7.06-7.00 (m, 2H), 4.32 (q, J=7.2 Hz, 2H), 4.17-4.11 (m, 2H), 3.90 (t, J=4.8 Hz, 2H), 3.66 (s, 3H), 3.50 (m, 2H), 3.52 (t, J=4.8 Hz, 2H), 3.14 (m, 2H), 1.18 (t, J=7.2 Hz, 3H).

### EXAMPLE 44

### 7-(2-Carbamoyl-4-fluoro-benzyl)-4-hydroxy-1-methyl-2-oxo-1,2-dihydro-[1,5]naphthyridine-3-carboxylic acid(2-hydroxy-ethyl)-amide

This compound was prepared from compound I1 in a manner similar to that described in Example 1 and was obtained at 98.0% yield:

m.p.: 250° C. (decomp.)

¹H NMR (DMSO-d6) δ 10.52(1H, bs), 8.39(1H, d, J=0.9 Hz), 7.95(1H, brs), 7.77(1H, s), 7.59-7.49(1H, m), 7.38-7.42(1H, m), 7.27-7.20(2H,m), 4.30(2H, s), 3.55-3.48(2H, m), 3.50(3H, s), 3.36-3.40(2H, m), 2.59 (1H, t, J=5.4 Hz).

### EXAMPLE 45

### 7-(2-Carbamoyl-4-fluoro-benzyl)-4-hydroxy-1-methyl-2-oxo-1,2-dihydro-[1,5]naphthyridine-3-carboxylic acid(2-methoxy-ethyl)-amide

This compound was prepared from compound I1 in a manner similar to that described in Example 1 and was obtained at 77.0% yield:

m.p.: 265° C.

¹H NMR (DMSO-d6) δ 10.41(1H, bs), 8.52(1H, s), 7.97-7.93(2H, m), 7.61(1H, s), 7.41-7.47(1H, m), 7.28-7.22(2H, m), 4.34(2H, s), 3.57 (3H,s), 3.56-3.50(4H, m), 3.30(3H, s).

### EXAMPLE 46

### 7-(4-Fluoro-2-methylcarbamoyl-benzyl)-4-hydroxy-1-methyl-2-oxo-1,2-dihydro-[1,5]naphthyridine-3-carboxylic acid(2-hydroxy-ethyl)-amide

This compound was prepared from compound I2 in a manner similar to that described in Example 1 and was obtained at 68.0% yield:

m.p.: 264-266° C.

Elemental Analysis: C₂₁H₂₁F₁N₄O₅

Calcd (%): C, 58.87; H, 4.94; N, 13.08; F, 4.43.

Found (%): C, 58.88; H, 4.96; N, 13.11; F, 4.25.

¹H NMR (DMSO-d6) δ 10.41(1H, bs), 8.49(1H, s), 8.41(1H, bs), 7.91(1H, s), 7.46-7.42(1H, m), 7.27-7.21(2H, m), 4.91(1H, bs), 4.28(2H, s), 3.57-3.54(5H, m), 3.46-3.43(2H, m), 2.72(3H, d, J=4.5 Hz).

### EXAMPLE 47

### 7-(4-Fluoro-2-methylcarbamoyl-benzyl)-4-hydroxy-1-methyl-2-oxo-1,2-dihydro-[1,5]naphthyridine-3-carboxylic acid(2-methoxy-ethyl)-amide

This compound was prepared from compound I2 in a manner similar to that described in Example 1 and was obtained at 71.0% yield:

m.p.: 252-254° C.

Elemental Analysis: C₂₂H₂₃F₁N₄O₅(H₂O)_{0.3}

Calcd (%): C, 59.00; H, 5.31; N, 12.51; F, 4.24.

Found (%): C, 59.11; H, 5.24; N, 12.27; F, 4.03.

¹H NMR (DMSO-d6) δ 10.40(1H, bs), 8.49(1H, s), 8.41(1H,bs), 7.92(1H, s), 7.40-7.48(1H, m), 7.27-7.21(2H, m), 4.28(2H, s), 3.56-3.59(7H, m), 3.30(3H, s), 2.72(3H, d, J=4.5 Hz).

### EXAMPLE 48

### 7-(2-Dimethylcarbamoyl-4-fluoro-benzyl)-4-hydroxy-1-methyl-2-oxo-1,2-dihydro-[1,5]naphthyridine-3-carboxylic acid(2-hydroxy-ethyl)-amide

This compound was prepared from compound I3 in a manner similar to that described in Example 1 and was obtained at 90.0% yield:

m.p.: 217-219° C.

HRMS (FAB, positive): C22H23FN4O5+H

Calcd: 443.1731. Found: 443.1739.

(DMSO-d6) δ: 2.61(1H, brt, J=5.7 Hz), 2.92(3H, s), 3.42(2H, m), 3.55(6H, s), 4.08(2H, brs), 4.55(0.4H, br), 4.93(0.6H, br), 7.16(1H, dd, J=3.0 Hz, 8.7 Hz), 7.23(1H, ddd, J=3.0 Hz, 8.7 Hz, 8.7 Hz), 7.48(1H, dd, J=5.4 Hz, 8.7 Hz), 7.84(1H, d, J=1.8 Hz), 8.40(1H, d, J=1.8 Hz).

### EXAMPLE 49

### 7-(2-Dimethylcarbamoyl-4-fluoro-benzyl)-4-hydroxy-1-methyl-2-oxo-1,2-dihydro-[1,5]naphthyridine-3-carboxylic acid(2-methoxy-ethyl)-amide

This compound was prepared from compound I3 in a manner similar to that described in Example 1 and was obtained at 58.0% yield:

m.p.: 217-219° C.

HRMS (FAB, positive): C22H23FN4O5+H

Calcd: 443.1731. Found: 443.1739.

(DMSO-d6) δ: 2.61(1H, brt, J=5.7 Hz), 2.92(3H, s), 3.42(2H, m), 3.55(6H, s), 4.08(2H, brs), 4.55(0.4H, br), 4.93(0.6H, br), 7.16(1H, dd, J=3.0 Hz, 8.7 Hz), 7.23(1H, ddd, J=3.0 Hz, 8.7 Hz, 8.7 Hz), 7.48(1H, dd, J =5.4 Hz, 8.7 Hz), 7.84(1H, d, J=1.8 Hz), 8.40(1H, d, J=1.8 Hz).

### EXAMPLE 50

### 7-[4-Fluoro-2-(4-methyl-piperazine-1-carbonyl)-benzyl]-4-hydroxy-1-methyl-2-oxo-1,2-dihydro-[1,5]naphthyridine-3-carboxylic acid(2-hydroxy-ethyl)-amide

This compound was prepared from compound I4 in a manner similar to that described in Example 1 and was obtained at 24.0% yield:

m.p.: 190° C.

¹H NMR (CDCl₃) δ10.52 (s, 1H), 8.48 (s, 1H), 7.70 (s, 1H), 7.20 (s, 2H), 7.07 (s, 1H), 6.93 (d, J=8.10 Hz, 1H), 4.15-4.00 (m, 2H), 3.90 (s, 2H), 3.84 (s, 2H), 3.80 (s, 3H), 3.21 (s, 2H), 2.93 (s, 2H), 2.40 (s, 2H), 2.24 (s, 3H), 2.00 (s, 2H).

### EXAMPLE 51

### 7-[4-Fluoro-2-(4-methyl-piperazine-1-carbonyl)-benzyl]-4-hydroxy-1-methyl-2-oxo-1,2-dihydro-[1,5]naphthyridine-3-carboxylic acid(2-methoxy-ethyl)-amide

This compound was prepared from compound I4 in a manner similar to that described in Example 1 and was obtained at 39.0% yield:

m.p.: 186-187° C.

¹H NMR (CDCl₃) δ 8.64 (s, 1H), 8.50 (s, 1H), 7.52 (s, 1H), 7.21 (s, 2H), 4.32 (s, 2H), 3.71 (s, 3H), 3.70-3.10 (m, 12H), 3.40 (s, 3H), 2.95 (s, 3H).

### EXAMPLE 52

### 7-[4-Fluoro-2-(morpholine-4-carbonyl)-benzyl]-4-hydroxy-1-methyl-2-oxo-1,2-dihydro-[1,5]naphthyridine-3-carboxylic acid(2-hydroxy-ethyl)-amide

This compound was prepared from compound I5 in a manner similar to that described in Example 1 and was obtained at 43.0% yield:

m.p.: 184-185° C.

FAB-MS: m/z 485 (M+1)

¹H NMR (CDCl₃) δ 10.47(1H, bs), 8.54(1H, s), 7.71(1H, s), 7.28-7.23(2H, m), 7.11-7.05(1H, m), 6.95(1H, dd, J=2.7, 8.7 Hz), 4.25-2.80(14H, m, br), 3.60(3H, s)

### EXAMPLE 53

### 7-[4-Fluoro-2-(morpholine-4-carbonyl)-benzyl]-4-hydroxy-1-methyl-2-oxo-1,2-dihydro-[1,5]naphthyridine-3-carboxylic acid(2-methoxy-ethyl)-amide

This compound was prepared from compound I5 in a manner similar to that described in Example 1 and was obtained at 29.0% yield:

m.p.: 163-164° C.

FAB-MS: m/z 499 (M+1)

¹H NMR (CDCl₃) δ 10.36(1H, br), 8.54(1H, s), 7.71(1H, s), 7.26-7.23(2H, m), 7.11-7.05(1H, m), 6.94(1H, dd, J=2.7, 8.1 Hz), 4.25-2.80(14H, m, br), 3.62(3H, s), 3.42(3H, s)

### EXAMPLE 54

### 7-(2-Ethylcarbamoyl-4-fluoro-benzyl)-4-hydroxy-1-methyl-2-oxo-1,2-dihydro-[1,5]naphthyridine-3-carboxylic acid(2-hydroxy-ethyl)-amide

This compound was prepared from compound I6 in a manner similar to that described in Example 1 and was obtained at 71.0% yield:

m.p.: 227-229° C.

Elemental Analysis: C₂₂H₂₃F₁N₄O₅

Calcd (%): C, 59.72; H, 5.24; N, 12.66; F, 4.29.

Found (%): C, 59.72; H, 5.17; N, 12.67; F, 4.27.

¹H NMR (DMSO-d6) δ 10.42(1H, bs), 8.47-8.42(2H, m), 7.88(1H, s), 7.46-7.41(1H, m), 7.25-7.19(2H, m), 4.92(1H, t, J=5.0 Hz), 4.29(2H, s), 3.56-3.53(5H, m), 3.48-3.40(2H, m), 3.25-3.10(2H, m), 1.00(3H, t, J=7.2 Hz).

### EXAMPLE 55

### 7-(2-Ethylcarbamoyl-4-fluoro-benzyl)-4-hydroxy-1-methyl-2-oxo-1,2-dihydro-[1,5]naphthyridine-3-carboxylic acid(2-methoxy-ethyl)-amide

This compound was prepared from compound I6 in a manner similar to that described in Example 1 and was obtained at 69.0% yield:

m.p.: 223-225° C.

Elemental Analysis: C₂₃H₂₅F₁N₄O₅

Calcd (%): C, 60.52; H, 5.52; N, 12.27; F, 4.16.

Found (%): C, 60.60; H, 5.50; N, 12.28; F, 3.92.

¹H NMR (DMSO-d6) δ 10.40(1H, bs), 8.48-8.45(2H, m), 7.89(1H, s), 7.46-7.42(1H, m), 7.25-7.19(2H, m), 4.29(2H, s), 3.56-3.50(7H, m), 3.30(3H,s), 3.25-3.18(2H, m), 1.00(3H, t, J=7.2 Hz).

### EXAMPLE 56

### 7-(2-Diethylcarbamoyl-4-fluoro-benzyl)-4-hydroxy-1-methyl-2-oxo-1,2-dihydro-[1,5]naphthyridine-3-carboxylic acid(2-hydroxy-ethyl)-amide

This compound was prepared from compound I7 in a manner similar to that described in Example 1 and was obtained at 73.0% yield:

m.p.: 156-157° C.

Elemental Analysis: C₂₄H₂₇F₁N₄O₅(H₂O)_{0.3}

Calcd (%): C, 60.57; H, 5.85; N, 11.77; F, 3.99.

Found (%): C, 60.46; H, 5.80; N, 11.77; F, 3.85.

¹H NMR (DMSO-d6) δ 10.42(1H, bs), 8.46(1H, s), 7.93(1H, s), 7.49-7.42(1H, m), 7.28-7.15(2H, m), 4.93(1H, t, J=5.1 Hz), 4.08(2H, bs), 3.57-3.54(5H, m), 3.47-3.40(2H, m), 2.95(2H, q, J=7.2 Hz), 1.10(3H, t, J=7.2 Hz), 0.95(3H, t, J=7.2 Hz).

### EXAMPLE 57

### 7-(2-Diethylcarbamoyl-4-fluoro-benzyl)-4-hydroxy-1-methyl-2-oxo-1,2-dihydro-[1,5]naphthyridine-3-carboxylic acid(2-methoxy-ethyl)-amide

This compound was prepared from compound I7 in a manner similar to that described in Example 1 and was obtained at 46.0% yield:

m.p.: 176-178° C.

Elemental Analysis: C₂₅H₂₉F₁N₄O₅

Calcd (%): C, 61.97; H, 6.03; N, 11.56; F, 3.92.

Found (%): C, 61.89; H, 6.02; N, 11.43; F, 3.66.

¹H NMR (DMSO-d6) δ 10.41(1H, bs), 8.46(1H, s), 7.93(1H, s), 7.49-7.42(1H, m), 7.28-7.14(2H, m), 4.09(2H, bs), 3.56-3.51(7H, m), 2.94(2H, q, J=7.2 Hz), 1.10(3H, t, J=6.9 Hz), 0.95(3H, t, J=7.2 Hz).

### EXAMPLE 58

### 7-(4-Fluoro-2-isopropylcarbamoyl-benzyl)-4-hydroxy-1-methyl-2-oxo-1,2-dihydro-[1,5]naphthyridine-3-carboxylic acid(2-hydroxy-ethyl)-amide

This compound was prepared from compound I8 in a manner similar to that described in Example 1 and was obtained at 65.0% yield:

m.p.: 212-215° C.

¹H NMR (DMSO-d6) δ 10.42(1H, bs), 8.41(1H, s), 8.32 (1H, d, J=7.8 Hz), 7.80(1H, s), 7.45-7.41(1H, m), 7.28-7.15(2H, m), 4.27(2H, s), 3.99-3.94(1H, m), 3.56-3.52(5H, m), 3.43-3.39(2H, m), 2.59 (1H, t, J=5.1 Hz), 1.04(6H, d, J=6.6 Hz).

### EXAMPLE 59

### 7-(4-Fluoro-2-isopropylcarbamoyl-benzyl)-4-hydroxy-1-methyl-2-oxo-1,2-dihydro-[1,5]naphthyridine-3-carboxylic acid(2-methoxy-ethyl)-amide

This compound was prepared from compound I8 in a manner similar to that described in Example 1 and was obtained at 78.0% yield:

m.p.: 235-236° C.

Elemental Analysis: C₂₄H₂₇F₁N₄O₅

Calcd (%): C, 61.27; H, 5.78; N, 11.91; F, 4.04.

Found (%): C, 61.14; H, 5.64; N, 11.96; F, 3.79.

¹H NMR (DMSO-d6) δ 10.40(1H, bs), 8.47(1H, s), 8.32 (1H, d, J=7.5 Hz), 7.88(1H, s), 7.46-7.41(1H, m), 7.28-7.16(2H, m), 4.29(2H, s), 4.00-3.94(1H, m), 3.56-3.50(7H, m), 3.30(3H,s), 1.04(6H, d, J=6.6 Hz).

### EXAMPLE 60

### 7-[2-(Ethyl-methyl-carbamoyl)-4-fluoro-benzyl]-4-hydroxy-1-methyl-2-oxo-1,2-dihydro-[1,5]naphthyridine-3-carboxylic acid(2-hydroxy-ethyl)-amide

This compound was prepared from compound I9 in a manner similar to that described in Example 1 and was obtained at 26.0% yield:

m.p.: 159-162° C.

¹H NMR (DMSO-d6) δ 10.42(1H, bs), 8.44(1H, s), 7.90(1H, s), 7.52-7.43(1H, m), 7.26-7.10(2H, m), 4.93(1H, t, J=5.1 Hz), 4.09(2H, bs), 3.57-3.53(5H, m), 3.45-3.36(2H, m), 3.33-2.58(5H, m), 1.11-1.02(3H, m).

### EXAMPLE 61

### 7-[2-(Ethyl-methyl-carbamoyl)-4-fluoro-benzyl]-4-hydroxy-1-methyl-2-oxo-1,2-dihydro-[1,5]naphthyridine-3-carboxylic acid(2-methoxy-ethyl)-amide

This compound was prepared from compound I9 in a manner similar to that described in Example 1 and was obtained at 24.0% yield:

m.p.: 128-131° C.

¹H NMR (CDCl₃) δ 10.37(1H, bs), 8.52(1H, s), 7.69-7.67(1H, m), 7.22-7.17(1H, m), 7.07-6.92(2H, m), 4.08(2H, s), 3.68-3.46(9H, m), 3.34(3H,s), 3.00(3H, s), 1.03(3H, t, J=6.9 Hz).

### EXAMPLE 62

### 7-{2-[Bis-(2-hydroxy-ethyl)-carbamoyl]-4-fluoro-benzyl}-4-hydroxy-1-methyl-2-oxo-1,2-dihydro-[1,5]naphthyridine-3-carboxylic acid(2-hydroxy-ethyl)-amide

This compound was prepared from compound I10 in a manner similar to that described in Example 1 and was obtained at 32.0% yield:

m.p.: 161-162° C.

¹H NMR (CDCl₃) δ10.45 (s, 1H), 8.46 (s, 1H), 7.86 (m, 1H), 7.45 (m, 2H), 7.17 (m, 1H), 4.95 (s, 2H), 4.08 (s, 2H), 3.80-3.10 (m, 10H), 3.59 (s, 3H).

### EXAMPLE 63

### 7-{2-[Bis-(2-hydroxy-ethyl)-carbamoyl]-4-fluoro-benzyl}-4-hydroxy-1-methyl-2-oxo-1,2-dihydro-[1,5]naphthyridine-3-carboxylic acid(2-methoxy-ethyl)-amide

This compound was prepared from compound I10 in a manner similar to that described in Example 1 and was obtained at 77.0% yield:

¹H NMR (CDCl₃) δ 10.44 (s, 1H), 8.50 (s, 1H), 7.94 (s, 1H), 7.84 (d, J=6.6 Hz, 1H), 7.52-7.36 (m, 2H), 7.21 (m, 2H), 4.82 (m, 2H), 4.09 (s, 2H), 3.80-3.10 (m, 10H), 3.58 (s, 3H), 3.10 (s, 3H).

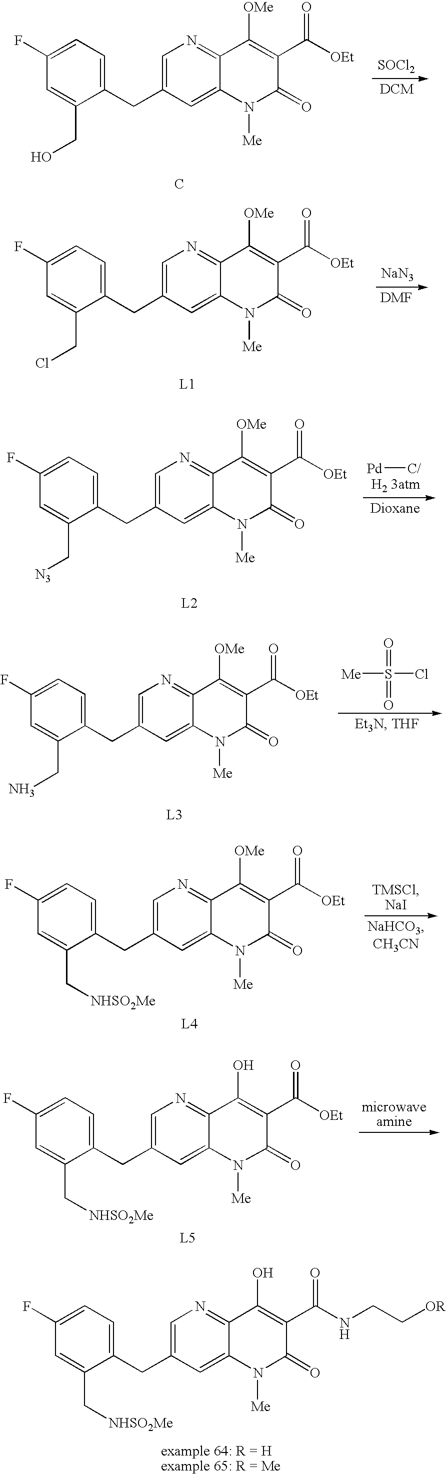

### Compound L1 7-(2-Chloromethyl-4-fluoro-benzyl)-4-methoxy-1-methyl-2-oxo-1,2-dihydro-[1,5]naphthyridine-3-carboxylic acid ethyl ester

A mixture of compound C (1.41 g, 3.52 mmol) in CH₂Cl₂ was treated with thionyl chloride (310 μL, 4.23 mmol) at 0° C. under N₂ atmosphere. The resulting mixture was stirred at rt for 10 min and was treated with ice/water. The product mixture was extracted three times with EtOAc, and the combined organic layer was washed with sat. NaHCO₃ solution, water and then brine. After dried over Na₂SO₄, the solvent was concentrated in vacuo to give pale yellow solid. The resulting solid was washed with diisopropylether to give 1.42 g of compound L1 as a colorless powder.

¹H NMR (CDCl₃) δ 8.47(1H, s), 7.37(1H, s), 7.17-7.00(3H, m), 4.53(2H, s), 4.45(2H, q, 7.2 Hz), 4.27(2H, s), 4.24(3H, s), 1.41(3H, t, 7.2 Hz)

### Compound L2 7-(2-Azidomethyl-4-fluoro-benzyl)-4-methoxy-1-methyl-2-oxo-1,2-dihydro-[1,5]naphthyridine-3-carboxylic acid ethyl ester

To a mixture of compound L1 (1.35 g, 3.22 mmol) in N,N-dimethylformamide was added sodium azide (251 mg, 3.87 mmol) at rt. The resulting mixture was stirred at rt for 1 hour, and then quenched with ice/water. The product mixture was extracted three times with CHCl₃, and the combined organic layer was washed with sat. NaHCO₃, water and then brine. After dried over Na₂SO₄, the solvent was concentrated in vacuo to provide 1.38 g of compound L2 as a yellow oil.

¹H NMR (CDCl₃) δ 8.44(1H, br), 7.32(1H, s), 7.17-7.03(3H, m), 4.45(2H, q, 7.2 Hz), 4.30(2H, s), 4.24(3H, s), 4.19(2H, s), 3.55 (3H, s), 1.41(3H, t, 7.2 Hz)

### Compound L3 7-(2-Aminomethyl-4-fluoro-benzyl)-4-methoxy-1-methyl-2-oxo-1,2-dihydro-[1,5]naphthyridine-3-carboxylic acid ethyl ester

A mixture of compound L2 (485 mg, 1.14 mmol) in 1,4-dioxane was treated with 10% palladium charcoal (wet. 50% water) (100 mg) at rt. The resulting mixture was stirred at rt under 3 atm of H₂ atmosphore for 1 h. The mixture was passed through a pad of celite, and the filtrate was concentrated under reduced pressure to afford compound L3 as yellow oil. The obtained residue was used for the next step without further purification.

ESI: m/z 400 (M+1)

### Compound L4 7-[4-Fluoro-2-(methanesulfonylamino-methyl)-benzyl]-4-methoxy-1-methyl-2-oxo-1,2-dihydro-[1,5]naphthyridine-3-carboxylic acid ethyl ester

A mixture of compound L3 (1.14 mmol) and Et₃N (794 μL, 5.7 mmol) in THF was treated with MsCl (265 μL, 3.42 mmol) at rt. After stirred at rt under N₂ atmosphere for 1 h, the mixture was treated with water. The product mixture was extracted three times with EtOAc, and the combined organic layer was washed sequentially with 2N HCl, sat. NaHCO₃ solution, water, and brine. After dried over Na₂SO₄, the solvent was concentrated in vacuo. The product mixture was purified by silicagel column chromatography (eluent: CHCl₃/MeOH=20/1) to give 275 mg of compound L4 as a colorless oil.

¹H NMR (CDCl₃) δ 8.33(1H, d, J=1.8), 7.49(1H, br), 7.21-7.01(3H, m), 4.62(1H, br), 4.45(2H, q, J=7.1 Hz), 4.24(2H, s), 4.22(3H, s), 4.22(2H, s), 3.58(3H, s), 2.93(3H, s), 1.41(3H, t, J=7.2 Hz)

### Compound L5 7-[4-Fluoro-2-(methanesulfonylamino-methyl)-benzyl]-4-hydroxy-1-methyl-2-oxo-1,2-dihydro-[1,5]naphthyridine-3-carboxylic acid ethyl ester

A solution of NaI (689 mg, 4.6 mmol) in MeCN (5 mL) was treated with trimethylsilyl chloride (584 μL, 4.6 mmol) at 0° C. The resulting mixture was added to a mixture of compound L4 (275 mg, 0.58 mmol) and NaHCO₃ (689 mg, 4.6 mmol) in MeCN (3 mL) at 0° C. After stirred at rt for 1 h, the mixture was treated with 10% aq. NaHSO₃. The product mixture was extracted three times with CHCl₃, dried over Na₂SO₄, and concentrated under reduced pressure. The obtained residue was used for the next step without further purification.

¹H NMR (CDCl₃) δ 8.43(1H, br), 7.50(1H, br), 7.21-7.01(3H, m), 4.74(1H, br), 4.51(2H, q, J=6.8 Hz), 4.24(4H, br), 3.57(3H, s), 2.94(3H, s), 1.48(3H, t, J=7.2 Hz)

### EXAMPLE 64

### 7-[4-Fluoro-2-(methanesulfonylamino-methyl)-benzyl]-4-hydroxy-1-methyl-2-oxo-1,2-dihydro-[1,5]naphthyridine-3-carboxylic acid(2-hydroxyethyl)-amide

This compound was prepared in the similar manner as Example 1

m.p.: 232-233° C.

FAB-MS: m/z 479 (M+1)

¹H NMR (DMSO-d6) δ 10.41(1H, t, J=5.7 Hz), 8.46(1H, s), 7.55(1H, t, J=6.6 Hz), 7.28-7.00(3H, m), 4.92(1H, t, J=5.1 Hz), 4.23-4.20(4H, m), 3.70-3.57(4H, m), 3.55(3H, s), 3.60-3.20(4H, m), 2.93(3H, s)

### EXAMPLE 65

### 7-[4-Fluoro-2-(methanesulfonylamino-methyl)-benzyl]-4-hydroxy-1-methyl-2-oxo-1,2-dihydro-[1,5]naphthyridine-3-carboxylic acid(2-methoxyethyl)-amide

This compound was prepared in the similar manner as Example 1

m.p.: 216-217° C.

FAB-MS: m/z 493 (M+1)

¹H NMR (CDCl₃) δ 10.36(1H, t, J=5.4 Hz), 8.25(1H, s), 7.50(1H, s), 7.20-7.00(3H, m), 5.53(1H, t, J=6.3 Hz), 4.26-4.20(4H, m), 3.70-3.57(4H, m), 3.57(3H, s), 3.42(3H, s), 2.93(3H, s)

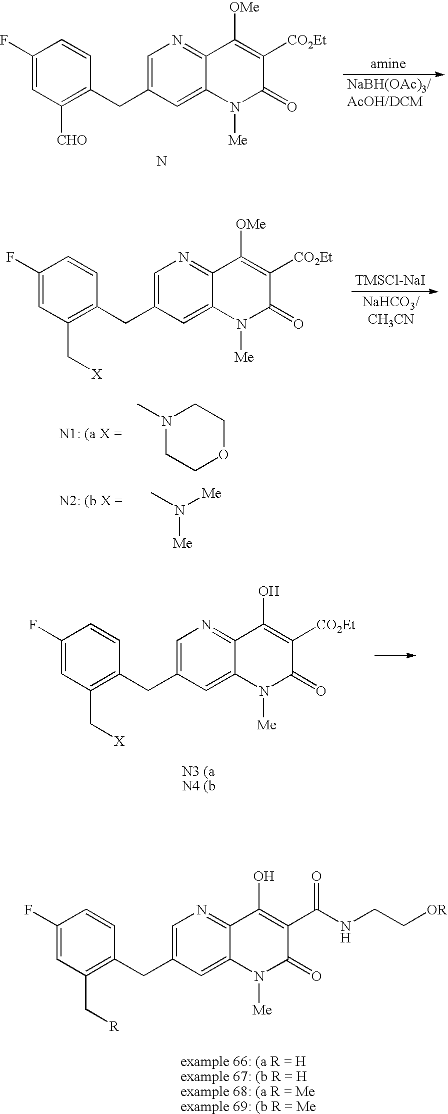

### Compound N1: 7-(4-Fluoro-2-morpholin-4-ylmethyl-benzyl)-4-methoxy-1-methyl-2-oxo-1,2-dihydro-[1,5]naphthyridine-3-carboxylic acid ethyl ester

To a solution of compound N (730 mg, 1.83 mmol) in CH₂Cl₂ (30 mL) were added successively morpholine (0.320 ml, 3.67 mmol), NaBH(Ac)₃ (777 mg, 3.67 mmol) and acetic acid (0.420 ml, 7.34 mmol) at 0° C., and mixture was stirred at rt for 1 h. After sat. NaHCO₃ solution was added to the reaction mixture, the resulting solution was extracted with CHCl₃. The extract was washed with brine, dried over Na₂SO₄, and the solvent was concentrated in vacuo. The residue was purified by silicagel column chromatography (eluent: toluene/acetone=3/1 v/v) to give 736 mg of compound N1 (yield=85.6%) as a colorless crystal.

¹H NMR (CDCl₃) δ 8.42(1H, d, J=1.8 Hz), 7.28(1H, d, J=1.8 Hz), 6.94-7.23(3H, m), 4.44(2H, q, J=7.2 Hz), 4.28(2H, s), 4.24(3H, s), 3.61(4H, t, J=4.5 Hz), 3.53(3H, s), 3.36(2H, s), 2.37(4H, t, J=4.5 Hz), 1.41(3H, t, J=7.2 Hz).

### Compound N2: 7-(2-Dimethylaminomethyl-4-fluoro-benzyl)-4-methoxy-1-methyl-2-oxo-1,2-dihydro-[1,5]naphthyridine-3-carboxylic acid ethyl ester

This compound was prepared from compound N in a manner similar to that described in compound N1 and was obtained at 80.9% yield:

¹H NMR (CDCl₃) δ 8.46(1H, d, J=1.8 Hz), 7.43(1H, d, J=1.8 Hz), 6.94-7.10 (3H, m), 4.44(2H, q, J=7.2 Hz), 4.26(2H, s), 4.24(3H, s), 3.54(3H, s), 3.36(2H, s), 2.23(6H, s), 1.41(3H, t, J=7.2 Hz).

### Compound N3: 7-(4-Fluoro-2-morpholin-4-ylmethyl-benzyl)-4-hydroxy-1-methyl-2-oxo-1,2-dihydro-[1,5]naphthyridine-3-carboxylic acid ethyl ester

To a mixture of compound N1 (733 mg, 1.56 mmol) and NaI (1.87 g, 12.48 mmol) in MeCN (29 mL) was added trimethylsilyl chloride (1.58 ml, 12.45 mmol) at 0° C., and the mixture was stirred for 15 min. After NaHCO₃ (1.05 g, 12.50 mmol) was added, the mixture was heated at reflux for 15 h. 10% NaHSO₃ solution (29 mL) was added to the mixture, and the resulting mixture was extracted three times with CHCl₃. The combined extracts were washed with brine, dried over Na₂SO₄. The solvent was concentrated in vacuo to afford the crude product. The crude product was purified by silicagel column chromatography (eluent: CHCl₃/MeOH=85/15 v/v) to afford 383 mg of compound N3 (yield=53.9%) as a yellow oil.

¹H NMR (CDCl₃) δ 8.48(1H, s), 7.25(1H, s), 6.94-7.15(3H, m), 4.52(2H, q, J=7.2 Hz), 1.48(3H, t, J=7.2 Hz).

### Compound N4: 7-(2-Dimethylaminomethyl-4-fluoro-benzyl)-4-hydroxy-1-methyl-2-oxo-1,2-dihydro-[1,5]naphthyridine-3-carboxylic acid ethyl ester

This compound was prepared from compound N2 in a manner similar to that described in compound N3 and was obtained at 44.0% yield:

¹H NMR (CDCl₃) δ 8.43(1H, s), 7.75(1H, bs), 7.09-7.21 (3H, m), 4.48-4.55(4H, m), 3.74(2H, bs), 3.60(3H, s), 2.64(6H, bs), 1.48(3H, t, J=7.2 Hz).

### EXAMPLE 66

### 7-(4-Fluoro-2-morpholin-4-ylmethyl-benzyl)-4-hydroxy-1-methyl-2-oxo-1,2-dihydro-[1,5]naphthyridine-3-carboxylic acid(2-hydroxy-ethyl)-amide

This compound was prepared from compound N3 in a manner similar to that described in Example 1 and was obtained at 46.2% yield:

m.p.: 208-209° C.

Elemental Analysis: C₂₄H₂₇F₁N₄O₅

Calcd (%): C, 61.27; H, 5.78; N, 11.91; F, 4.04.

Found (%): C, 61.16; H, 5.76; N, 11.70; F, 3.88.

¹H NMR (CDCl₃) δ 10.47(1H, bs), 8.55(1H, s), 7.30(1H, s), 6.96-7.13(3H, m), 4.32(2H, s), 3.83-3.89(2H, m), 3.55-3.70(9H, m), 3.37(2H, s), 2.37(4H, bs), 2.25(1H, bs).

### EXAMPLE 67

### 7-(2-Dimethylaminomethyl-4-fluoro-benzyl)-4-hydroxy-1-methyl-2-oxo-1,2-dihydro-[1,5]naphthyridine-3-carboxylic acid(2-hydroxy-ethyl)-amide

This compound was prepared from compound N4 in a manner similar to that described in Example 1 and was obtained at 23.9% yield:

m.p.: 162-164° C.

¹H NMR (CDCl₃) δ 10.48(1H, bs), 8.57(1H, s), 7.50(1H, brs), 6.95-7.12(3H, m), 4.31(2H, s), 3.84-3.88(2H, m), 3.62-3.68(2H, m), 3.56(3H, s), 3.39(2H, brs), 2.26(6H, bs).

### EXAMPLE 68

### 7-(4-Fluoro-2-morpholin-4-ylmethyl-benzyl)-4-hydroxy-1-methyl-2-oxo-1,2-dihydro-[1,5]naphthyridine-3-carboxylic acid(2-methoxy-ethyl)-amide

This compound was prepared from compound N3 in a manner similar to that described in Example 1 and was obtained at 27.8% yield:

m.p.: 130-132° C.

Elemental Analysis: C₂₅H₂₉F₁N₄O₅

Calcd (%): C, 61.97; H, 6.03; N, 11.56; F, 3.92.

Found (%): C, 61.81; E, 6.08; N, 11.49; F, 3.92.

¹H NMR (CDCl₃) δ 10.34(1H, bs), 8.54(1H, s), 7.28(1H, s), 6.95-7.13(3H, m), 4.31(2H, s), 3.54-3.69(11H, m), 3.42(3H, s), 3.36(2H, s), 2.37(4H, bs).

### EXAMPLE 69

### 7-(2-Dimethylaminomethyl-4-fluoro-benzyl)-4-hydroxy-1-methyl-2-oxo-1,2-dihydro-[1,5]naphthyridine-3-carboxylic acid(2-methoxy-ethyl)-amide

This compound was prepared from compound N4 in a manner similar to that described in Example 1 and was obtained at 29.0% yield:

m.p.: 122-124° C.

¹H NMR (CDCl₃) δ 10.37(1H, bs), 8.55(1H, s), 7.50(1H, brs), 6.95-7.11 (3H, m), 4.33(2H, s), 3.57-3.68(7H, m), 3.42(3H, s), 3.38(2H, bs), 2.28(6H, bs).

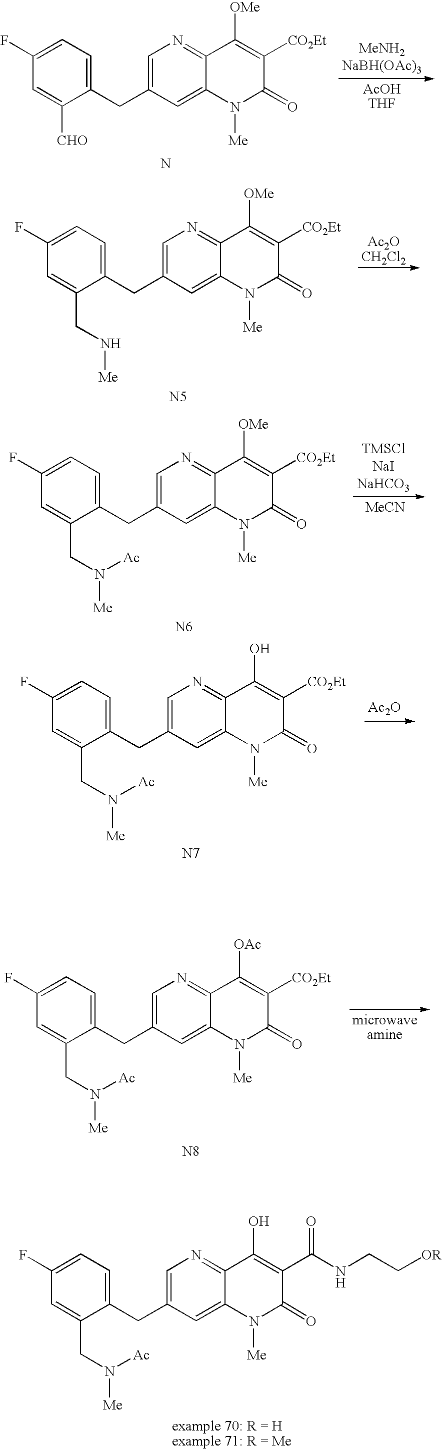

### Compound N5: 7-(4-Fluoro-2-methylaminomethyl-benzyl)-4-methoxy-1-methyl-2-oxo-1,2-dihydro-[1,5]naphthyridine-3-carboxylic acid ethyl ester

This compound was prepared from compound N (2.0 g, 5.02 mmol) in a manner similar to that described in compound N1 and was obtained at 63.0% yield as a colorless crystal:

¹H NMR (CDCl₃) δ 8.43(1H, s), 7.44(1H, s), 7.17-7.04(2H, m), 6.99-6.90(1H, m), 4.43(2H, q, J=7.2 Hz), 4.24(2H, s), 3.71(2H, s), 3.55(3H, s), 2.45(3H, s), 1.41(3H, t, J=7.2 Hz).

### Compound N6: 7-{2-[(Acetyl-methyl-amino)-methyl]-4-fluoro-benzyl}-4-methoxy-1-methyl-2-oxo-1,2-dihydro-[1,5]naphthyridine-3-carboxylic acid ethyl ester

To a solution of compound N5 (875 mg, 2.12 mmol) in CH₂Cl₂ (10 mL) was added acetic anhydride (150 μL) at 0° C., the mixture was stirred at rt for 1 h. After removal of the solvent, the residue was purified by silicagel column chromatography (eluent: CHCl₃/MeOH=100/0 to 90/10 v/v) to give 950 mg of compound N6 (yield=99.0%) as yellow form.

¹H NMR (CDCl₃) δ 8.40-8.35(1H, m), 7.45(1H, s), 7.19-7.15(1H, m), 7.03-6.90(2H, m), 4.57(2H, s), 4.48-4.43(2H, m), 4.25-4.23(3H, m), 4.17(2H, s), 3.60-3.55(3H, m), 2.86-2.86(3H, m), 2.03-1.99(3H, m), 1.44-1.39(3H, m).

### Compound N7: 7-{2-[(Acetyl-methyl-amino)-methyl]-4-fluoro-benzyl}-4-hydroxy-1-methyl-2-oxo-1,2-dihydro-[1,5]naphthyridine-3-carboxylic acid ethyl ester

This crude compound was prepared from compound N6 (950 mg, 2.09 mmol) in a manner similar to that described in compound N3:

### Compound N8: 4-Acetoxy-7-{2-[(acetyl-methyl-amino)-methyl]-4-fluoro-benzyl}-1-methyl-2-oxo-1,2-dihydro-[1,5]naphthyridine-3-carboxylic acid ethyl ester

This compound was prepared from aboved-mentioned crude compound N7 in a manner similar to that described in Example 1 and was obtained at 30% yield from compound N6 as a colorless crystal.

¹H NMR (CDCl₃) δ 8.28(1H, d, J=1.7 Hz), 7.50(1H, s), 7.20-7.11(1H, m), 7.04-6.98(2H, m), 4.53(2H, s), 4.43(2H, q, J=7.2 Hz), 4.15(2H, s), 3.64(3H, s), 2.80(3H, s), 2.41(3H, s), 1.96(3H, s), 1.38(3H, t, J=7.2 Hz).

### EXAMPLE 70

### 7-{2-[(Acetyl-methyl-amino)-methyl]-4-fluoro-benzyl}-4-hydroxy-1-methyl-2-oxo-1,2-dihydro-[1,5]naphthyridine-3-carboxylic acid(2-hydroxy-ethyl)-amide

This compound was prepared from compound N8 (160 mg, 0.33 mmol) in a manner similar to that described in Example 1 and was obtained at 46.0% yield:

m.p.: 200-202° C.

Elemental Analysis: C₂₃H₂₅F₁N₄O₅

Calcd (%): C, 60.52; H, 5.52; N, 12.27; F, 4.16.

Found (%): C, 60.45; H, 5.38; N, 12.25; F, 3.91.

¹H NMR (DMSO-d6) δ 10.43(1H, bs), 8.42(1H, s), 7.84(1H, s), 7.29-7.25(1H, m), 7.14-6.93(2H, m), 4.93(1H, bs), 4.54(2H, s), 4.19(2H, s), 3.57-3.54(5H, m), 3.45-3.43(2H, m), 2.89(3H, s), 2.03(3H, s).

### EXAMPLE 71

### 7-{2-[(Acetyl-methyl-amino)-methyl]-4-fluoro-benzyl}-4-hydroxy-1-methyl-2-oxo-1,2-dihydro-[1,5]naphthyridine-3-carboxylic acid(2-methoxy-ethyl)-amide

This compound was prepared from compound N8 (149 mg, 0.31 mmol) in a manner similar to that described in Example 1 and was obtained at 65.0% yield:

m.p.: 148-149° C.

Elemental Analysis: C₂₄H₂₇F₁N₄O₅

Calcd (%): C, 61.27; H, 5.78; N, 11.91; F, 4.04.

Found (%): C, 60.69; H, 5.86; N, 11.67; F, 3.89.

¹H NMR (DMSO-d6) δ 10.36(1H, bs), 8.41(1H, s), 7.78(1H, s), 7.29-7.24(1H, m), 7.09-6.83(2H, m), 4.55(2H, s), 4.21(2H, s), 3.56(3H, s), 3.56-3.52(2H, m), 3.31(3H, s), 3.10-3.08(2H, m), 2.88(3H, s), 2.01(3H, s).

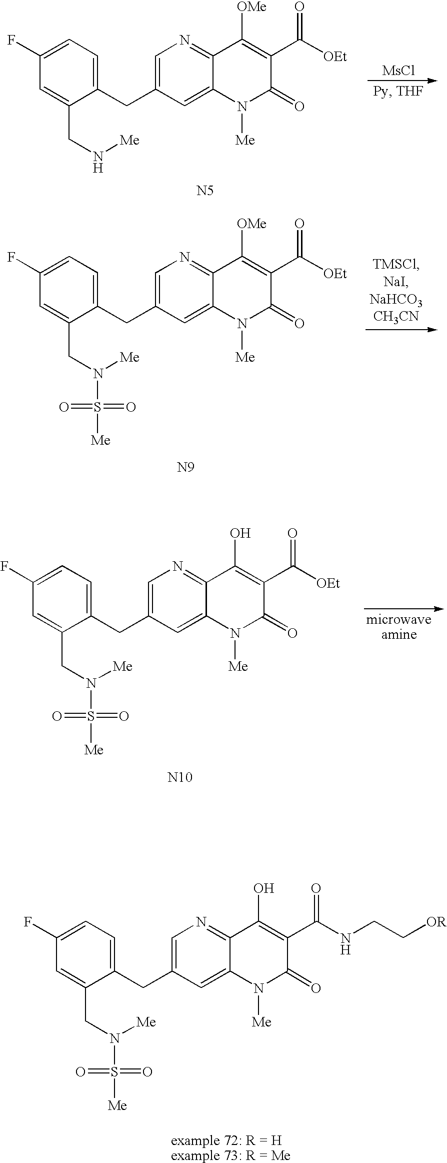

### Compound N9: 7-{4-Fluoro-2-[(methanesulfonyl-methyl-amino)-methyl]-benzyl}-4-methoxy-1-methyl-2-oxo-1,2-dihydro-[1,5]naphthyridine-3-carboxylic acid ethyl ester

To a solution of compound N5 (452 mg, 5.47 mmol) in THF (5 mL) were added MsCl (254 □l, 3.28 mmol) and pyridine (442 □l, 5.47 mmol) at rt, and the mixture was stirred for 5 h. The reaction mixture was cooled to 0° C., and extracted with EtOAc. The extract was washed with sat. NaHCO₃ solution and brine, and dried over Na₂SO₄. The solvent was concentrated in vacuo to afford the crude product. The crude product was purified by silicagel column chromatography (eluent: CHCl₃) to give 475.2 mg of compound N9 (yield=89.0%) as a clorless solid.

¹H NMR (CDCl₃) δ 8.36 (1H, s), 7.58 (1H, s), 7.27-7.21 (1H, m), 7.10-7.01 (2H, m), 4.44 (2H, q, J=7.2 Hz), 4.28(2H, s), 4.22(3H, s), 4.19(2H, s), 3.60(3H, s), 2.84(3H, s), 2.65(3H,s), 1.41 (3H, t, J=7.2 Hz).

### Compound N10: 7-{4-Fluoro-2-[(methanesulfonyl-methyl-amino)-methyl]-benzyl}-4-hydroxy-1-methyl-2-oxo-1,2-dihydro-[1,5]naphthyridine-3-carboxylic acid ethyl ester

To a solution of NaI (1.45 g, 9.66 mmol) in MeCN (10 mL) was added trimethylsilyl chloride (1.23 ml, 9.66 mmol) at 0° C., and the mixture was stirred for 10 min. The solution was added to a mixture of compound N9 (475.0 mg, 0.97 mmol) and NaHCO₃ (406.0 mg, 4.84 mmol) in MeCN (5 mL) at 0° C., the mixture was stirred at rt for 15 h. A solution of sat. NaHCO₃ was added to the reaction mixture at 0° C., and the resulting mixture was extracted with EtOAc. The extract was washed with brine, dried over Na₂SO₄. The solvent was concentrated in vacuo to afford the crude product. The crude product was purified by silicagel column chromatography (eluent: CHCl₃) to give 439.6 mg of compound N10 (yield=95.0%) as a colorless solid.

¹H NMR (CDCl₃) δ 8.40 (1H, s), 7.56 (1H, s), 7.27-7.21 (1H, m), 7.10-7.01 (2H, m), 4.45 (2H, q, J=7.2 Hz), 4.30(2H, s), 4.20(2H, s), 3.58(3H, s), 2.85(3H, s), 2.66(3H,s), 1.48 (3H, t, J=7.2 Hz).

### EXAMPLE 72

### 7-{4-Fluoro-2-[(methanesulfonyl-methyl-amino)-methyl]-benzyl}-4-hydroxy-1-methyl-2-oxo-1,2-dihydro-[1,5]naphthyridine-3-carboxylic acid(2-hydroxy-ethyl)-amide

This compound was prepared from compound N10 in a manner similar to that described in Example 1:

m.p.: 176° C.

¹H NMR (CDCl₃) δ 10.50(1H, bs), 8.43(1H, s), 7.65(1H, s), 7.30-7.25(1H, m), 7.13-7.00(2H, m), 4.31(2H, s), 4.20(2H, s), 3.89(2H, t, J=5.3 Hz), 3.66-3.64(2H, m), 3.62 (3H, s), 2.85(3H, s), 2.65 (3H, s).

### EXAMPLE 73

### 7-{4-Fluoro-2-[(methanesulfonyl-methyl-amino)-methyl]-benzyl}-4-hydroxy-1-methyl-2-oxo-1,2-dihydro-[1,5]naphthyridine-3-carboxylic acid(2-methoxy-ethyl)-amide

This compound was prepared from compound N10 in a manner similar to that described in Example 1:

¹H NMR (CDCl₃) δ 10.38(1H, bs), 8.45(1H, s), 7.64(1H, s), 7.30-7.25(1H, m), 7.13-7.00(2H, m), 4.30(2H, s), 4.19(2H, s), 3.67-3.50(4H, m), 3.63 (3H, s), 3.42 (3H, s), 2.84(3H, s), 2.64 (3H, s).

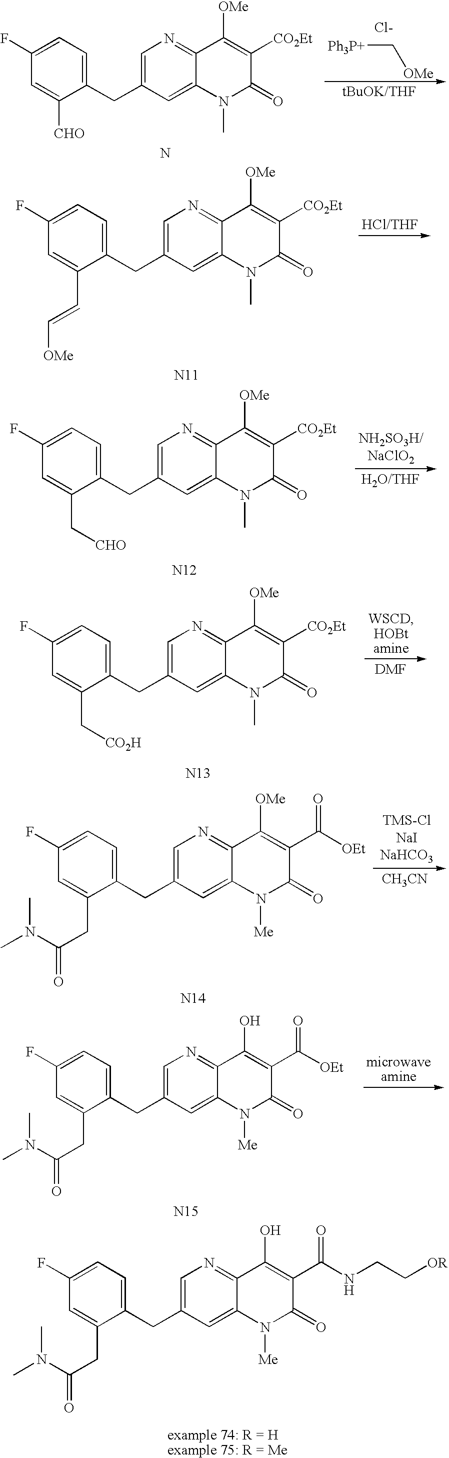

### Compound N11: 7-[4-Fluoro-2-(2-methoxy-vinyl)-benzyl]-4-methoxy-1-methyl-2-oxo-1,2-dihydro-[1,5]naphthyridine-3-carboxylic acid ethyl ester

To a solution of (methoxymethyl)triphenylphosphonium chloride (4.59 g, 13.4 mmol) in THF (40 mL) was added t-BuOK (1.35 g, 12.1 mmol) at 0° C., and the mixture was stirred for 1 h. To a solution of compound N (1.33 g, 3.35 mmol) in THF (15 mL) was added dropwise above-mentioned solution (30 mL) at 0° C., the the mixture was stirred for 30 min. The reaction mixture was quenched with 10% citric acid solution, and extracted with EtOAc. The extract was washed with water and brine, and dried over Na₂SO₄. The solvent was concentrated in vacuo, and the residue was purified by silicagel column chromatography (eluent: n-Hexane/EtOAc=2/1 v/v) to afford the crude product of compound N11.

### Compound N12: 7-[4-Fluoro-2-(2-oxo-ethyl)-benzoyl]-4-methoxy-1-methyl-2-oxo-1,2-dihydro-[1,5]naphthyridine-3-carboxylic acid ethyl ester

A mixture of compound N11 in THF (10 mL) and 2N HCl (10 mL) was heated at 70° C. for 40 min with stirring. After cooling to rt, water was added to the mixture, and then extracted with CHCl₃. The extract was washed with water and brine, dried oner Na₂SO₄. The solvent was concentrated in vacuo, and the residue was purified by silicagel column chromatography (eluent: EtOAc) to afford the crude product of compound N12.

### Compound N13: 7-(2-Carboxymethyl-4-fluoro-benzyl)-4-methoxy-1-methyl-2-oxo-1,2-dihydro-[1,5]naphthyridine-3-carboxylic acid ethyl ester

To a solution of sulfamide (971 mg, 10 mmol) and NaClO₄ (904 mg, 10 mmol) in water (9 mL) was added dropwise a solution of compound N12 in THF (54 mL) at rt, and the reaction mixture was stirred for 30 min. After water and EtOAc were added to the mixture, separated EtOAc layer was washed with brine, dried over Na₂SO₄. The solvent was concentrated in vacuo, and the residue was purified by silicagel column chromatography (eluent: EtOAc) to give 789 mg of compound N13 (yield 55% from compound N).

¹H NMR (DMSO-d6) δ 8.43(1H, s), 7.84(1H, s), 7.34-7.29(1H, m), 7.22-7.12(2H, m), 4.32 (2H, q, J=7.2 Hz), 4.23(2H, s), 4.19(3H, s), 3.79(2H, s), 3.57(3H, s), 1.19(3H, t, J=7.2 Hz).

### Compound N14: 7-(2-Dimethylcarbamoylmethyl-4-fluoro-benzyl)-4-methoxy-1-methyl-2-oxo-1,2-dihydro-[1,5]naphthyridine-3-carboxylic acid ethyl ester

This compound was prepared from compound N13 in a manner similar to that described in compound D6:

¹H NMR (CDCl₃) δ 8.34(1H, d, J=1.8 Hz), 7.38 (1H, d, J=1.8 Hz), 7.10-7.05(1H, m), 6.99-6.89(2H, m), 4.44(2H, q, J=7.2 Hz), 4.24(3H, s), 4.12(2H, s), 3.60(2H, 2), 3.54(3H, s), 2.97(3H, s), 2.89(3H,s), 1.41(3H, t, J=7.2 Hz).

### Compound N15: 7-(2-Dimethylearbamoylmethyl-4-fluoro-benzyl)-4-hydroxy-1-methyl-2-oxo-1,2-dihydro-[1,5]naphthyridine-3-carboxylic acid ethyl ester

This compound was prepared from compound N14 in a manner similar to that described in compound L5, and purified by silicagel column chromatography (eluent: CHCl₃/EtOAc) and crystallization to give compound N15 (yield=42.0%):

¹H NMR (CDCl₃) δ 8.50(1H, d, J=1.8 Hz), 7.36(1H, m), 7.10(1H, m), 6.99-6.91(2H, m), 4.51(2H, q, J=7.2 Hz), 4.14(2H, s), 3.60(2H, s), 3.53(3H, s), 2.98(3H, s), 2.87(3H, s), 1.48(3H, t, J=7.2 Hz).

### EXAMPLE 74

### 7-(2-Dimethylcarbamoylmethyl-4-fluoro-benzyl)-4-hydroxy-1-methyl-2-oxo-1,2-dihydro-[1,5]naphthyridine-3-carboxylic acid(2-hydroxy-ethyl)-amide

This compound was prepared from compound N15 in a manner similar to that described in Example 1:

m.p.: 231.5-232.0° C.

HRMS(FAB): C₂₃H₂₆FN₄O₅+H. Calcd: 457.1887. Found: 457.1893 (Int 100%).

¹H NMR (CDCl₃) δ 10.51(1H, brt), 8.57(1H, d, J=1.8 Hz), 7.41(1H, d, J=1.8 Hz), 7.10(1H, dd, J=5.7 Hz, 8.4 Hz), 7.00-6.90(2H, m), 4.15(2H, s), 3.86(2H, J=5.4 Hz, 4.8 Hz), 3.65(2H, m), 3.60(2H, s), 3.56(3H, s), 2.97(3H, s), 2.85(3H, s), 2.19(1H, t, J=5.4 Hz).

### EXAMPLE 75

### 7-(2-Dimethylcarbamoylmethyl-4-fluoro-benzyl)-4-hydroxy-1-methyl-2-oxo-1,2-dihydro-[1,5]naphthyridine-3-carboxylic acid(2-methoxy-ethyl)-amide

This compound was prepared from compound N15 in a manner similar to that described in Example 1:

m.p.: 206-207° C.

HRMS(FAB): C₂₄H₂₈FN₄O₅+H. Calcd: 471.2044. Found: 471.2047 (Int 100%).

¹H NMR (CDCl₃) δ 10.37(1H, brt), 8.56(1H, d, J=1.8 Hz), 7.39(1H, m), 7.10(1H, dd, J=5.7 Hz, 8.4 Hz), 6.99-6.91(2H, m), 4.15(2H, s), 3.65(2H, m), 3.59(2H, m), 3.59(2H, s), 3.55(3H, s), 3.42(3H, s), 2.96(3H, s), 2.85(3H, s).

### EXAMPLE 76

### Biological Activity

The inhibitory activities of the present compounds against integrase were determined by the assay described below.

### (1) Preparation of DNA Solutions

A substrate DNA solution (2 pmol/μl) and a target DNA solution (5 pmol/μl) were prepared by the same method as that described in Experimental Example 1 of WO 2004/024693. The DNA solutions were heated and then slowly cooled so that they were annealed with each complement. Each sequence of the substrate DNA and the target DNA is the same as described in the Experimental Example.

### (2) Calculations of the Percent Inhibitions (IC₅₀ Values of Test Compounds)

Streptavidin, obtained from Vector Laboratories, was dissolved in 0.1 M carbonate buffer (composition: 90 mM Na₂CO₃, 10 mM NaHCO₃) at a concentration of 40 μg/ml. After coating each well of microtiter plates (obtained from NUNC) with 50 μl of the above solution at 4° C. over night, each well was washed twice with PBS (composition: 13.7 mM NaCl, 0.27 mM KCl, 0.43 mM Na₂HPO₄, 0.14 mM KH₂PO₄) and blocked with 300 μl of 1% skim milk in PBS for 30 min. Additionally, each well was washed twice with PBS and added with 50 μl of the substrate DNA solution (2 pmol/μl). The microtiter plates were kept at room temperature for 30 min. Then, each well was washed twice with PBS and once with H₂O.

Subsequently, to the each well prepared above were added 51 μl of the reaction buffer prepared from 12 μl of the buffer (composition: 150 mM MOPS (pH 7.2), 75 mM MnCl₂, 50 mM 2-mercaptoethanol, 25% glycerol, 500 μg/ml bovine serum albumin-fraction V) and 39 μl of the distilled water. Additionally, 9 μl of an integrase solution (30 pmol) was added thereto and the mixture was mixed well. To the well of negative control (NC) was added 9 μl of the integrase dilution buffer (composition: 20 mM MOPS (pH7.2), 400 mM potassium glutamate, 1 mM EDTA, 0.1% NP-40, 20% glycerol, 1 mM DTT, 4 M urea).

The microtiter plates were incubated at 30° C. for 1 hour. The reaction solution was removed and each well was washed three times with 250 μl of the wash buffer (composition: 150 mM MOPS (pH 7.2), 50 mM 2-mercaptoethanol, 25% glycerol, 500 μg/ml bovine serum albumin-fraction V).

Subsequently, to each well prepared above were added 53 μl of the reaction buffer prepared from 12 μl of the buffer (composition: 150 mM MOPS (pH 7.2), 75 mM MgCl₂, 50 mM 2-mercaptoethanol, 25% glycerol, 500 μg/ml bovine serum albumin-fraction V) and 41 μl of the distilled water. Additionally, 6 μl of either a test compound in DMSO or DMSO for positive control (PC) was mixed well. After the microtiter plates were incubated at 30° C. for 30 minutes, 1 μl of target DNA (5 pmol/μl) was added to each well, and the reaction mixture was mixed well.

The microtiter plates were incubated at 30° C. for 10 minutes. The reaction solution was removed and each well was washed twice with PBS. Subsequently, each well of the microtiter plates was filled with 100 μl of anti-digoxigenin antibody labeled with alkaline phosphatase (Sheep Fab fragment: obtained from Boehringer) and incubated at 30° C. for 1 hour. Then, each well was washed twice with 0.05% Tween20 in PBS and once with PBS. Next, 150 μl of the Alkaline phosphatase reaction buffer (composition: 10 mM p-Nitrophenylphosphate (obtained from Vector Laboratories), 5 mM MgCl₂, 100 mM NaCl, 100 mM Tris-HCl (pH 9.5)) was added to each well. The microtiter plates were incubated at 30° C. for 2 hours and the reaction was terminated by the addition of 50 μl of 1 N NaOH solution. The optical density (OD) at 405 nm of each well was measured and the percent inhibition was determined by the following expression.

The percent inhibition (%)=100[1−{(C abs.−NC abs.)/(PC abs.−NC abs.)}]

C abs.; the OD of the well of the test compound

NC abs.: the OD of the negative control (NC)

PC abs.: the OD of the positive control (PC)

When the percent inhibition (%) is X % at a concentration of ×μg/ml and the percent inhibition (%) is Y % at a concentration of y μg/ml, one of which is more than 50% and the other is less than 50%, IC₅₀ can be determined by the following expression.

IC₅₀(μg/ml)=x−{(X−50)(x−y)/(X−Y)}

### Results

Compounds of the present invention have integrase inhibition activity in the range IC₅₀=1−1000 nM. For example, compounds of examples 1, 2, 9, 13, 15, 17, 22, 25, 33 and 72 have integrase inhibition activity with an IC₅₀ less than 100 nM.

### EXAMPLE 77

### Biological Activity

### MT4 Assay

MT-4 cells (human T-cell line established by HTLV-I-induced transformation) were cultured in RPMI1640 medium supplemented with 10% fetal calf serum and kanamycin (60 mg/ml) in 5% CO₂, humidified incubator at 37° C. The stock of HIV-1 was produced as follows. Molt-4 cells persistently infected with HIV-1 (IIIB strain) were cultured for 3 days and culture supernatant was collected. After removal of cells by centrifugation, the supernatant was passed through membrane filter (0.45 mm) and stored as cell-free culture supernatant at −80° C. The infectivity of the stock virus was measured by MTT assay.

Anti-HIV activity in vitro of test compounds was assayed by inhibition of virus-induced CPE in MT-4 cells. MT-4 cells were suspended in culture medium at 2.5×10⁵ cells/ml. The cell suspension (100 ml) was added to each well of a 96 well flat-bottom microtiter plate containing serial 2-fold dilution of test compounds (50 ml/well). HIV-1 (50 ml/well) was added to each well (4-10 TCID₅₀/well). After 4-day incubation at 37° C., the viability of MT-4 cells was determined by the MTT method using 3-(4,5-dimethylthiazol-2-yl)-2,5-diphenyltetrazolium bromide.

The absorbance (OD) of each well was measured at two wavelengths (560 and 690 nm). All data represent the average values of three wells. The concentration (conc.) achieving 50% protection of HIV replication (EC₅₀) were calculated.

### Results

Compounds of the present invention have an anti-HIV activity with an EC₅₀ less than 1000 nM. For example, compounds of Examples 1, 2, 9, 13, 15, 17, 22, 25, 33 and 72 have an EC₅₀ in the range of 1-100 nM.

What is claimed is:

1. A compound of formula (I) 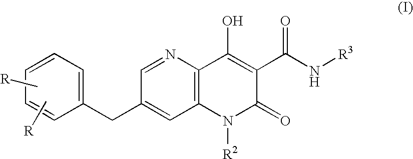
wherein:
R is halogen;
R¹ is
(a) C(O)N(R^{a}R^{b});
(b) C₃₋₆ alkynyl or C₁₋₈ alkylcarbonyl, each of which may be optionally substituted with one or more substituents independently selected from the group consisting of C₁₋₈ alkyl, C₁₋₈ haloalkyl, C₃₋₇ cycloalkyl, C₂₋₆ alkenyl, C₃₋₇ cycloalkenyl, C₃₋₆ alkynyl, halogen, CN, NO₂, OR^{a}, N(R^{a}R^{b}), S(O)ₘR^{a}, SR^{a}, OS(O)ₘR^{a}, S(O)ₘOR^{a}, OS(O)ₘOR^{a}, N(R^{a})S(O)ₘR^{b}, S(O)ₘN(R^{a}R^{b}), N(R^{a})S(O)ₘN(R^{a}R^{b}), OS(O)ₘN(R^{a}R^{b}), N(R^{a})S(O)ₘOR^{b}, C(O)R^{a}, OC(O)R^{a}, C(O)OR^{a}, OC(O)OR^{a}, N(R^{a})C(O)R^{b}, C(O)N(R^{a}R^{b}), N(R^{a})C(O)N(R^{a}R^{b}), OC(O)N(R^{a}R^{b}), N(R^{a})C(O)OR^{b}, C(NR^{a}R^{b})═N(R^{a}), N(R^{a})C(NR^{a}R^{b})═N(R^{a}), C(SR^{a})═N(R^{b}), C(OR^{a})═N(R^{b}), N(R^{a})C(SR^{a})═N(R^{b}) and heterocycle optionally substituted with oxo or R^{a}; or
(c) C₁₋₈ alkyl or C₁₋₈ alkoxy, each of which is substituted with one or more substituents independently selected from the group consisting of C₆₋₁₄ aryl, C₁₋₈ alkyl, C₁₋₈ haloalkyl, C₃₋₇ cycloalkyl, C₂₋₆ alkenyl, C₃₋₇ cycloalkenyl, C₃₋₆ alkynyl, halogen, CN, NO₂, OR^{a}, N(R^{a}R^{b}), S(O)ₘR^{a}, SR^{a}, OS(O)ₘR^{a}, S(O)ₘOR^{a}, OS(O)ₘOR^{a}, N(R^{a})S(O)ₘR^{b}, S(O)ₘN(R^{a}R^{b}), N(R^{a})S(O)ₘN(R^{a}R^{b}), OS(O)ₘN(R^{a}R^{b}), N(R^{a})S(O)ₘOR^{b}, C(O)R^{a}, OC(O)R^{a}, C(O)OR^{a}, OC(O)OR^{a}, N(R^{a})C(O)R^{b}, C(O)N(R^{a}R^{b}), N(R^{a})C(O)N(R^{a}R^{b}), OC(O)N(R^{a}R^{b}), N(R^{a})C(O)OR^{b}, C(NR^{a}R^{b})═N(R^{a}), N(R^{a})C(NR^{a}R^{b})═N(R^{a}), C(SR^{a})═N(R^{b}), C(OR^{a})═N(R^{b}), N(R^{a})C(SR^{a})═N(R^{b}) and heterocycle optionally substituted with oxo or R^{a},
R² is hydrogen, C₁₋₈ alkyl, C₁₋₈ haloalkyl, C₃₋₇ cycloalkyl, C₆₋₁₄ aralkyl, C₂₋₆ alkenyl, C₃₋₇ cycloalkenyl, C₃₋₆ alkynyl, C₆₋₁₄ aryl, or heterocycle, each of which may be optionally substituted with one or more substituents independently selected from the group consisting of C₁₋₈ alkyl, C₁₋₈ haloalkyl, C₃₋₇ cycloalkyl, C₂₋₆ alkenyl, C₃₋₇ cycloalkenyl, C₃₋₆ alkynyl, halogen, CN, NO₂, OR^{a}, N(R^{a}R^{b}), S(O)ₘR^{a}, SR^{a}, OS(O)ₘR^{a}, S(O)ₘOR^{a}, OS(O)ₘOR^{a}, N(R^{a})S(O)ₘR^{b}, S(O)ₘN(R^{a}R^{b}), N(R^{a})S(O)ₘN(R^{a}R^{b}), OS(O)ₘN(R^{a}R^{b}), N(R^{a})S(O)ₘOR^{b}, C(O)R^{a}, OC(O)R^{a}, C(O)OR^{a}, OC(O)OR^{a}, N(R^{a})C(O)R^{b}, C(O)N(R^{a}R^{b}), N(R^{a})C(O)N(R^{a}R^{b}), OC(O)N(R^{a}R^{b}), N(R^{a})C(O)OR^{b}, C(NR^{a}R^{b})═N(R^{a}), N(R^{a})C(NR^{a}R^{b})═N(R^{a}), C(SR^{a})═N(R^{b}), C(OR^{a})═N(R^{b}), N(R^{a})C(SR^{a})═N(R^{b}) and heterocycle optionally substituted with oxo or R^{a};
or optionally when R² is C₅₋₇ cycloalkyl, C₆₋₁₄ aralkyl, C₅₋₇ cycloalkenyl, C₆₋₁₄ aryl or heterocycle R² may be fused to 5-7 membered carbocyclic or heterocyclic rings;
R³ is hydrogen, hydroxy, C₁₋₈ alkyl, C₁₋₈ haloalkyl, C₃₋₇ cycloalkyl, C₂₋₆ alkenyl, C₃₋₇ cycloalkenyl, C₃₋₆ alkynyl, N(R^{a}R^{b}), or heterocycle, each of which may be optionally substituted with one or more substituents independently selected from the group consisting of C₁₋₈ alkyl, C₁₋₈ haloalkyl, C₃₋₇ cycloalkyl, C₂₋₆ alkenyl, C₃₋₇ cycloalkenyl, C₃₋₆ alkynyl, halogen, oxo, CN, NO₂, OR^{a}, N(R^{a}R^{b}), S(O)ₘR^{a}, SR^{a}, OS(O)ₘR^{a}, S(O)ₘOR^{a}, OS(O)ₘOR^{a}, N(R^{a})S(O)ₘR^{b}, S(O)ₘN(R^{a}R^{b}), N(R^{a})S(O)ₘN(R^{a}R^{b}), OS(O)ₘN(R^{a}R^{b}), N(R^{a})S(O)ₘOR^{b}, C(O)R^{a}, OC(O)R^{a}, C(O)OR^{a}, OC(O)OR^{a}, N(R^{a})C(O)R^{b}, C(O)N(R^{a}R^{b}), N(R^{a})C(O)N(R^{a}R^{b}), OC(O)N(R^{a}R^{b}), N(R^{a})C(O)OR^{b}, C(NR^{a})═N(R^{b}), C(SR^{a})═N(R^{b}), C(OR^{a})═N(R^{b}), N(R^{a})C(NR^{a}R^{b})═N(R^{a}), N(R^{a})C(SR^{a})═N(R^{b}), N(R^{a})C(OR^{a})═N(R^{b}) and heterocycle optionally substituted by oxo or R^{a};
R^{a} and R^{b} are independently hydrogen, NO₂, OR^{c}, CN, N(R^{c}R^{d}), C(O)R^{c}, C(O)C(O)R^{c}, C(O)N(R^{c}R^{d}), C(O)C(O)N(R^{c}R^{d}), S(O)ₘR^{c}, SR^{c}, S(O)ₘN(R^{c}R^{d}), C₁₋₈ alkyl, C₁₋₈ haloalkyl, C₃₋₇ cycloalkyl, C₆₋₁₄ aralkyl, C₂₋₆ alkenyl, C₃₋₇ cycloalkenyl, C₃₋₆ alkynyl, C₆₋₁₄ aryl or heterocycle, each of which may be optionally substituted with one or more substituents independently selected from the group consisting of C₁₋₈ alkyl, C₁₋₈ haloalkyl, C₃₋₇ cycloalkyl, C₆₋₁₄ aralkyl, C₂₋₆ alkenyl, C₃₋₇ cycloalkenyl, C₃₋₆ alkynyl, C₆₋₁₄ aryl, CN, NO₂, OR^{c}, N(R^{c}R^{d}), S(O)ₘR^{c}, SR^{c}, OS(O)ₘR^{c}, S(O)ₘOR^{c}, OS(O)ₘOR^{c}, N(R^{c})S(O)ₘR^{d}, S(O)ₘN(R^{c}R^{d}), N(R^{c})S(O)ₘN(R^{c}R^{d}), OS(O)ₘN(R^{c}R^{d}), N(R^{c})S(O)ₘOR^{d}, C(O)R^{c}, OC(O)R^{c}, C(O)OR^{c}, OC(O)OR^{c}, N(R^{c})C(O)R^{d}, C(O)N(R^{c}R^{d}), N(R^{c})C(O)N(R^{c}R^{d}), OC(O)N(R^{c}R^{d}), N(R^{c})C(O)OR^{d}, C(NR^{c}R^{d})═N(R^{c}), C(SR^{c})═N(R^{d}), C(OR^{c})═N(R^{d}) and heterocycle;
Optionally, R^{a} and R^{b} may be linked together through one or more ring carbon atoms and/or ring heteroatoms including N, O, C(R^{c}R^{d}), C(O), S(O)ₘ, or S to form a saturated or unsaturated 3 to 8 membered carbocyclic or heterocyclic ring optionally substituted with oxo or R^{a};
R^{c} and R^{d} are independently hydrogen, C₁₋₈ alkyl, C₁₋₈ haloalkyl, C₃₋₇ cycloalkyl, C₆₋₁₄ aralkyl, C₂₋₆ alkenyl, C₃₋₇ cycloalkenyl, C₃₋₆ alkynyl, C₆₋₁₄ aryl or heterocycle;
Optionally, R^{c} and R^{d} may be linked together through one or more ring carbon atoms and/or ring heteroatoms including N, O, C(O) and S(O)ₘ, or S to form a saturated or unsaturated 3 to 8 membered carbocyclic or heterocyclic ring;
m is 1 or 2;
or a pharmaceutically acceptable salt thereof.

2. A compound of formula (I) according to claim 1 wherein:
R is halogen;
R¹ is
(a) C₁₋₈alkoxy substituted with C₆₋₁₄aryl; OR^{a}; or NR^{a}R^{b};
(b) C(O)NR^{a}R^{b};
(c) C₃₋₆alkynyl substituted with OR^{a} or NR^{a}R^{b};
(d) C₁₋₈alkylcarbonyl; or
(e) C₁₋₈alkyl substituted with OR^{a}; C(O)NR^{a}R^{b}; N(R^{a})S(O)ₘR^{b}; N(R^{a})C(O)R^{b}; NR^{a}R^{b}; or heterocycle optionally substituted with one or more oxo;
R² is C₁₋₈alkyl;
R³ is
(a) C₁₋₈alkyl optionally substituted with C₁₋₈alkyl, C₃₋₇cycloalkyl, OR^{a}, SR^{a}, C(O)N(R^{a}R^{b}), NR^{a}C(O)R^{b}, or heterocycle optionally substituted with oxo or R^{a};
(b) C₃₋₇cycloalkyl;
(c) C₁₋₈haloalkyl; or
(d) heterocycle optionally substituted with oxo;
R^{a} and R^{b} are independently hydrogen, NO₂, OR^{c}, CN, N(R^{c}R^{d}), C(O)R^{c}, C(O)C(O)R^{c}, C(O)N(R^{c}R^{d}), C(O)C(O)N(R^{c}R^{d}), S(O)ₘR^{c}, SRC, S(O)ₘN(R^{c}R^{d}), C₁₋₈ alkyl, C₁₋₈ haloalkyl, C₃₋₇ cycloalkyl, C₆₋₁₄ aralkyl, C₂₋₆ alkenyl, C₃₋₇ cycloalkenyl, C₃₋₆ alkynyl, C₆₋₁₄ aryl or heterocycle, each of which may be optionally substituted with one or more substituents independently selected from the group consisting of C₁₋₈ alkyl, C₁₋₈ haloalkyl, C₃₋₇ cycloalkyl, C₆₋₁₄ aralkyl, C₂₋₆ alkenyl, C₃₋₇ cycloalkenyl, C₃₋₆ alkynyl, C₆₋₁₄ aryl, CN, NO₂, OR^{c}, N(R^{c}R^{d}), S(O)ₘR^{c}, SR^{c}, OS(O)ₘR^{c}, S(O)ₘOR^{c}, OS(O)ₘOR^{c}, N(R^{c})S(O)ₘR^{d}, S(O)ₘN(R^{c}R^{d}), N(R^{c})S(O)ₘN(R^{c}R^{d}), OS(O)ₘN(R^{c}R^{d}) N(R^{c})S(O)ₘOR^{d}, C(O)R^{c}, OC(O)R^{c}, C(O)OR^{c}, OC(O)OR^{c}, N(R^{c})C(O)R^{d}, C(O)N(R^{c}R^{d}), N(R^{c})C(O)N(R^{c}R^{d}), OC(O)N(R^{c}R^{d}), N(R^{c})C(O)OR^{d}, C(NR^{c}R_{d})═N(R^{c}), C(SR^{c})═N(R^{d}), C(OR^{c})═N(R^{d}) and heterocycle;
Optionally, R^{a} and R^{b} may be linked together through one or more ring carbon atoms and/or ring heteroatoms including N, O, C(R^{c}R^{d}), C(O), S(O)ₘ, or S to form a saturated or unsaturated 3 to 8 membered carbocyclic or heterocyclic ring optionally substituted with oxo or R^{a};
R^{c} and R^{d} are independently hydrogen, C₁₋₈ alkyl, C₁₋₈ haloalkyl, C₃₋₇ cycloalkyl, C₆₋₁₄ aralkyl, C₂₋₆ alkenyl, C₃₋₇ cycloalkenyl, C₃₋₆ alkynyl, C₆₋₁₄ aryl or heterocycle;
Optionally, R^{c} and R^{d} may be linked together through one or more ring carbon atoms and/or ring heteroatoms including N, O, C(O) and S(O)ₘ, or S to form a saturated or unsaturated 3 to 8 membered carbocyclic or heterocyclic ring;
m is 1 or 2;
or a pharmaceutically acceptable salt thereof.

3. A compound of formula (I) according to claim 1 wherein:
R is halogen;
R¹ is
(a) C₁₋₈alkoxy substituted with C₆₋₁₄aryl; OR^{a} (wherein R^{a} is C₁₋₈alkyl); or NR^{a}R^{b} (wherein R^{a} and R^{b} are independently hydrogen or C(O)R^{c} wherein R^{c} is C₁₋₈alkyl);
(b) C(O)NR^{a}R^{b} wherein R^{a} and R^{b} are independently hydrogen, C₁₋₈ alkyl, or C(O)R^{c} wherein R^{c} is C₁₋₈alkyl;
(c) C₃₋₆alkynyl substituted with OR^{a} (wherein R^{a} is C₁₋₈alkyl) or NR^{a} R^{b} (wherein R^{a} and R^{b} are independently hydrogen or C(O)R^{c} wherein R^{c} is C₁₋₈alkyl);
(d) C₁₋₈alkylcarbonyl; or
(e) C₁₋₈alkyl substituted with OR^{a} (wherein R^{a} is hydrogen or C(O)N(R^{c}R^{d}) wherein R^{c} and R^{d} are independently hydrogen or C₁₋₈alkyl); C(O)NR^{a}R^{b} (wherein R^{a} and R^{b} are hydrogen, C₁₋₈alkyl, CN or OR^{c} wherein R^{c} is hydrogen); N(R^{a})S(O)ₘR^{b} (wherein R^{a} and R^{b} are independently hydrogen or C₁₋₈alkyl and m is 2); N(R^{a})C(O)R^{b} (wherein R^{a} and R^{b} are C₁₋₈alkyl); NR^{a}R^{b} (wherein R^{a} and R^{b} are independently hydrogen, C₁₋₈alkyl or C(O)R^{c} wherein R^{c} is C₁₋₈alkyl); or heterocycle optionally substituted with one or more oxo;
R² is C₁₋₈alkyl;
R³ is C₁₋₈alkyl optionally substituted with OR^{a} (wherein R^{a} is hydrogen or C₁₋₈alkyl);
or a pharmaceutically acceptable salt thereof.

4. A compound of formula (I) according to claim 1 wherein
R is halogen;
R¹ is C₁₋₈alkoxy substituted with C₆₋₁₄aryl; OR^{a} (wherein R^{a} is C₁₋₈alkyl); or NR^{a}R^{b} (wherein R^{a} and R^{b} are independently hydrogen or C(O)R^{c} wherein R^{c} is C₁₋₈alkyl);
R² is C₁₋₈alkyl;
R³ is C₁₋₈alkyl optionally substituted with OR^{a} (wherein R^{a} is hydrogen or C₁₋₈alkyl);
or a pharmaceutically acceptable salt thereof.

5. A compound of formula (Ia) 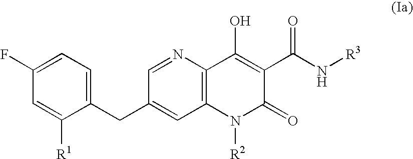
wherein:
R¹ is
(a) C(O)N(R^{a}R^{b}),
(b) C₃₋₆ alkynyl or C₁₋₈ alkylcarbonyl, each of which may be optionally substituted with one or more substituents independently selected from the group consisting of C₁₋₈ alkyl, C₁₋₈ haloalkyl, C₃₋₇ cycloalkyl, C₂₋₆ alkenyl, C₃₋₇ cycloalkenyl, C₃₋₆ alkynyl, halogen, CN, NO₂, OR^{a}, N(R^{a}R^{b}), S(O)ₘR^{a}, SR^{a}, OS(O)ₘR^{a}, S(O)ₘOR^{a}, OS(O)ₘOR^{a}, N(R^{a})S(O)ₘR^{b}, S(O)ₘN(R^{a}R), N(R^{a})S(O)ₘN(R^{a}R^{b}), OS(O)ₘN(R^{a}R^{b}), N(R^{a})S(O)ₘOR^{b}, C(O)R^{a}, OC(O)R^{a}, C(O)OR^{a}, OC(O)OR^{a}, N(R^{a})C(O)R^{b}, C(O)N(R^{a}R^{b}), N(R^{a})C(O)N(R^{a}R^{b}), OC(O)N(R^{a}R^{b}), N(R^{a})C(O)OR^{b}, C(NR^{a}R^{b})═N(R^{a}), N(R^{a})C(NR^{a}R^{b})═N(R^{a}), C(SR^{a})═N(R^{b})C(OR^{a})═N(R^{b}), N(R^{a})C(SR^{a})═N(R^{b}) and heterocycle optionally substituted with oxo or R^{a}, or
(c) C₁₋₈ alkyl or C₁₋₈ alkoxy, each of which is substituted with one or more substituents independently selected from the group consisting of C₆₋₁₄ aryl, C₁₋₈ alkyl, C₁₋₈ haloalkyl, C₃₋₇ cycloalkyl, C₂₋₆ alkenyl, C₃₋₇ cycloalkenyl, C₃₋₆ alkynyl, halogen, CN, NO₂, OR^{a}, N(R^{a}R^{b}), S(O)ₘR^{a}, SR^{a}, OS(O)ₘR^{a}, S(O)ₘOR^{a}, OS(O)ₘOR^{a}, N(R^{a})S(O)ₘR^{b}, S(O)ₘN(R^{aR b), N(R}^{a})S(O)ₘN(R^{a}R^{b}), OS(O)ₘN(R^{a}R^{b}), N(R^{a})S(O)ₘOR^{b}, C(O)R^{a}, OC(O)R^{a}, C(O)OR^{a}, OC(O)OR^{a}, N(R^{a})C(O)R^{b}, C(O)N(R^{a}R^{b}), N(R^{a})C(O)N(R^{a}R^{b}), OC(O)N(R^{a}R^{b}), N(R^{a})C(O)OR^{b}, C(NR^{a}R^{b})═N(R^{a}), N(R^{a})C(NR^{a}R^{b})═N(R^{a}), C(SR^{a})═N(R^{b}), C(OR^{a})═N(R^{b}), N(R^{a})C(SR^{a})═N(R^{b}) and heterocycle optionally substituted with oxo or R^{a};
R² is hydrogen, C₁₋₈ alkyl, C₁₋₈ haloalkyl, C₃₋₇ cycloalkyl, C₆₋₁₄ aralkyl, C₂₋₆ alkenyl, C₃₋₇ cycloalkenyl, C₃₋₆ alkynyl, C₆₋₁₄ aryl or heterocycle, each of which may be optionally substituted with one or more substituents independently selected from the group consisting of C₁₋₈ alkyl, C₁₋₈ haloalkyl, C₃₋₇ cycloalkyl, C₂₋₆ alkenyl, C₃₋₇ cycloalkenyl, C₃₋₆alkynyl, halogen, CN, NO₂, OR^{a}, N(R^{a}R^{b}), S(O)ₘR^{a}, SR^{a}, OS(O)ₘR^{a}, S(O)ₘOR^{a}, OS(O)ₘOR^{a}, N(R^{a})S(O)ₘR^{b}, S(O)ₘN(R^{a}R^{b}), N(R^{a})S(O)ₘN(R^{a}R^{b}), OS(O)ₘN(R^{a}R^{b}), N(R^{a})S(O)ₘOR^{b}, C(O)R^{a}, OC(O)R^{a}, C(O)OR^{a}, OC(O)OR^{a}, N(R^{a})C(O)R^{b}, C(O)N(R^{a}R^{b}), N(R^{a})C(O)N(R^{a}R^{b}), OC(O)N(R^{a}R^{b}), N(R^{a})C(O)OR^{b}, C(NR^{a}R^{b})═N(R^{a}), N(R^{a})C(NR^{a}R^{b})═N(R^{a}), C(SR^{a})═N(R^{b}), C(OR^{a})═N(R^{b}), N(R^{a})C(SR^{a})═N(R^{b}) and heterocycle optionally substituted with oxo or R^{a};
or optionally when R² is C₅₋₇ cycloalkyl, C₆₋₁₄ aralkyl, C₅₋₇ cycloalkenyl, C₆₋₁₄ aryl or heterocycle R² may be fused to 5-7 membered carbocyclic or heterocyclic rings;
R³ is hydrogen, hydroxy, C₁₋₈ alkyl, C₁₋₈ haloalkyl, C₃₋₇ cycloalkyl, C₂₋₆ alkenyl, C₃₋₇ cycloalkenyl, C₃₋₆ alkynyl, N(R^{a}R^{b}), or heterocycle, each of which may be optionally substituted with one or more substituents independently selected from the group consisting of C₁₋₈ alkyl, C₁₋₈ haloalkyl, C₃₋₇ cycloalkyl, C₂₋₆ alkenyl, C₃₋₇ cycloalkenyl, C₃₋₆ alkynyl, halogen, oxo, CN, NO₂, OR^{a}, N(R^{a}R^{b}), S(O)ₘR^{a}, SR^{a}, OS(O)ₘR^{a}, S(O)ₘOR^{a}, OS(O)ₘOR^{a}, N(R^{a})S(O)ₘR^{b}, S(O)ₘN(R^{a}R^{b}), N(R^{a})S(O)ₘN(R^{a}R^{b}), OS(O)ₘN(R^{a}R^{b}), N(R^{a})S(O)ₘOR^{b}, C(O)R^{a}, OC(O)R^{a}, C(O)OR^{a}, OC(O)OR^{a}, N(R^{a})C(O)R^{b}, C(O)N(R^{a}R^{b}), N(R^{a})C(O)N(R^{a}R^{b}), OC(O)N(R^{a}R^{b}), N(R^{a})C(O)OR^{b}, C(NR^{a})═N(R^{b}), C(SR^{a})═N(R^{b}), C(R^{a})═N(R^{b}), N(R^{a})C(NR^{a}R^{b})═N(R^{a}), N(R^{a})C(SR^{a})═N(R^{b}), N(R^{a})C(OR^{a})═N(R^{b}), and heterocycle optionally substituted by oxo or R^{a};
R^{a} and R^{b} are independently hydrogen, NO₂, OR^{c}, CN, N(R^{c}R^{d}), C(O)R^{c}, C(O)C(O)R^{c}, C(O)N(R^{c}R^{d}), C(O)C(O)N(R^{c}R^{d}), S(O)ₘR^{c}, SR^{c}, S(O)ₘN(R^{c}R^{d}), C₁₋₈ alkyl, C₁₋₈ haloalkyl, C₃₋₇ cycloalkyl, C₆₋₁₄ aralkyl, C₂₋₆ alkenyl, C₃₋₇ cycloalkenyl, C₃₋₆ alkynyl, C₆₋₁₄ aryl or heterocycle, each of which may be optionally substituted with one or more substituents independently selected from the group consisting of C₁₋₈ alkyl, C₁₋₈ haloalkyl, C₃₋₇ cycloalkyl, C₆₋₁₄ aralkyl, C₂₋₆ alkenyl, C₃₋₇ cycloalkenyl, C₃₋₆ alkynyl, C₆₋₁₄ aryl, CN, NO₂, OR^{c}, N(R^{c}R^{d}), S(O)ₘR^{c}, SR^{c}, OS(O)ₘR^{c}, S(O)ₘOR^{c}, OS(O)ₘOR^{c}, N(R^{c})S(O)ₘR^{d}, S(O)ₘN(R^{c}R^{d}), N(R^{c})S(O)ₘN(R^{c}R^{d}), OS(O)ₘN(R^{c}R^{d}), N(R^{c})S(O)ₘOR^{d}, C(O)R^{c}, OC(O)R^{c}, C(O)OR^{c}, OC(O)OR^{c}, N(R^{c})C(O)R^{d}, C(O)N(R^{c}R^{d}), N(R^{c})C(O)N(R^{c}R^{d}), OC(O)N(R^{c}R^{d}), N(R^{c})C(O)OR^{d}, C(NR^{c}R^{d})N(R^{c}), C(SR^{c})═N(R^{d}), C(OR^{c})═N(R^{d}) and heterocycle;
Optionally, R^{a} and R^{b} may be linked together through one or more ring carbon atoms and/or ring heteroatoms including N, O, C(R^{c}R^{d}), C(O), S(O)ₘ, or S to form a saturated or unsaturated 3 to 8 membered carbocyclic or heterocyclic ring optionally substituted with oxo or R^{a};
R^{c} and R^{d} are independently hydrogen, C₁₋₈ alkyl, C₁₋₈ haloalkyl, C₃₋₇ cycloalkyl, C₆₋₁₄ aralkyl, C₂₋₆ alkenyl, C₃₋₇ cycloalkenyl, C₃₋₆ alkynyl, C₆₋₁₄ aryl or heterocycle;
Optionally, R^{c} and R^{d} may be linked together through one or more ring carbon atoms and/or ring heteroatoms including N, O, C(O) and S(O)ₘ, or S to form a saturated or unsaturated 3 to 8 membered carbocyclic or heterocyclic ring;
m is 1 or 2;
or a pharmaceutically acceptable salt thereof.

6. A compound of formula (la) according to claim 5 wherein:
R¹ is
(a) C₁₋₈alkoxy substituted with C₆₋₁₄aryl; OR^{a} or NR^{a}R^{b};
(b) C(O)NR^{a}R^{b};
(c) C₃₋₆alkynyl substituted with OR^{a} or NR^{a}R^{b};
(d) C₁₋₈alkylcarbonyl; or
(e) C₁₋₈alkyl substituted with OR^{a}; C(O)NR^{a}R^{b}; N(R^{a})S(O)ₘR^{b}; N(R^{a})C(O)R^{b}; NR^{a}R^{b}; or heterocycle optionally substituted with one or more oxo;
R² is C₁₋₈alkyl;
R³ is
(a) C₁₋₈alkyl optionally substituted with C₁₋₈alkyl, C₃₋₇cycloalkyl, OR^{a}, SR^{a}, C(O)N(R^{a}R^{b}), NR^{a}C(O)R^{b}, or heterocycle optionally substituted with oxo or R^{a};
(b) C₃₋₇cycloalkyl;
(c) C₁₋₈haloalkyl; or
(d) heterocycle optionally substituted with oxo;
R^{a} and R^{b} are independently hydrogen, NO₂, OR^{c}, CN, N(R^{c}R^{d}), C(O)R^{c}, C(O)C(O)R^{c}, C(O)N(R^{c}R^{d}), C(O)C(O)N(R^{c}R^{d}), S(O)ₘR^{c}, SR^{c}, S(O)ₘN(R^{c}R^{d}), C₁₋₈ alkyl, C₁₋₈ haloalkyl, C₃₋₇ cycloalkyl, C₆₋₁₄ aralkyl, C₂₋₆ alkenyl, C₃₋₇ cycloalkenyl, C₃₋₆ alkynyl, C₆₋₁₄ aryl or heterocycle, each of which may be optionally substituted with one or more substituents independently selected from the group consisting of C₁₋₈ alkyl, C₁₋₈ haloalkyl, C₃₋₇ cycloalkyl, C₆₋₁₄ aralkyl, C₂₋₆ alkenyl, C₃₋₇ cycloalkenyl, C₃₋₆ alkynyl, C₆₋₁₄ aryl, CN, NO₂, OR^{c}, N(R^{c}R^{d}), S(O)ₘR^{c}, SR^{c}, OS(O)ₘR^{c}, S(O)ₘOR^{c}, OS(O)ₘOR^{c}, N(R^{c})S(O)ₘR^{d}, S(O)ₘN(R^{c}R^{d}), N(R^{c})S(O)ₘN(R^{c}R^{d}), OS(O)ₘN(R^{c}R^{d}), N(R^{c})S(O)ₘOR^{d}, C(O)R^{c}, OC(O)R^{c}, C(O)OR^{c}, OC(O)OR^{c}, N(R^{c})C(O)R^{d}, C(O)N(R^{c}R^{d}), N(R^{c})C(O)N(R^{c}R^{d}), OC(O)N(R^{c}R^{d}), N(R^{c})C(O)OR^{d}, C(NR^{c}R^{d})N(R^{c}), C(SR^{c})═N(R^{d}), C(OR^{c})═N(R^{d}) and heterocycle;
Optionally, R^{a} and R^{b} may be linked together through one or more ring carbon atoms and/or ring heteroatoms including N, O, C(R^{c}R^{d}), C(O), S(O)ₘ, or S to form a saturated or unsaturated 3 to 8 membered carbocyclic or heterocyclic ring optionally substituted with oxo or R^{a};
R^{c} and R^{d} are independently hydrogen, C₁₋₈ alkyl, C₁₋₈ haloalkyl, C₃₋₇ cycloalkyl, C₆₋₁₄ aralkyl, C₂₋₆ alkenyl, C₃₋₇ cycloalkenyl, C₃₋₆ alkynyl, C₆₋₁₄ aryl or heterocycl
Optionally, R^{c} and R^{d} may be linked together through one or more ring carbon atoms and/or ring heteroatoms including N, O, C(O) and S(O)ₘ, or S to form a saturated or unsaturated 3 to 8 membered carbocyclic or heterocyclic ring;
m is 1 or 2;
or a pharmaceutically acceptable salt thereof

7. A compound of formula (la) according to claim 5 wherein:
R¹ is
(a) C₁₋₈alkoxy substituted with C₆₋₁₄aryl; OR^{a} (wherein R^{a} is C₁₋₈alkyl); or NR^{a}R^{b} (wherein R^{a} and R^{b} are independently hydrogen or C(O)R^{c} wherein R^{c} is C₁₋₈alkyl);
(b) C(O)NR^{a}R^{b} wherein R^{a} and R^{b} are independently hydrogen, C₁₋₈ alkyl, or C(O)R^{c} wherein R^{c} is C₁₋₈alkyl;
(c) C₃₋₆alkynyl substituted with OR^{a} (wherein R^{a} is C₁₋₈alkyl) or NR^{a} R^{b} (wherein R^{a} and R^{b} are independently hydrogen or C(O)R^{c} wherein R^{c} is C₁₋₈alkyl);
(d) C₁₋₈alkylcarbonyl; or
(e) C₁₋₈alkyl substituted with OR^{a} (wherein R^{a} is hydrogen or C(O)N(R^{c}R^{d}) wherein R^{c} and R^{d} are independently hydrogen or C₁₋₈alkyl); C(O)NR^{a}R^{b} (wherein R^{a} and R^{b} are hydrogen, C₁₋₈alkyl, CN or OR^{c} wherein R^{c} is hydrogen); N(R^{a})S(O)ₘR^{b} (wherein R^{a} and R^{b} are independently hydrogen or C₁₋₈alkyl and m is 2); N(R^{a})C(O)R^{b} (wherein R^{a} and R^{b} are C₁₋₈alkyl); NR^{a}R^{b} (wherein R^{a} and R^{b} are independently hydrogen, C₁₋₈alkyl or C(O)R^{c} wherein R^{c} is C₁₋₈alkyl); or heterocycle optionally substituted with one or more oxo;
R² is C₁₋₈alkyl;
R³ is C₁₋₈alkyl optionally substituted with OR^{a} wherein R^{a} is hydrogen or C₁₋₈alkyl;
or a pharmaceutically acceptable salt thereof.

8. A compound of formula (Ia) according to claim 5 wherein:
R¹ is
(a) C₁₋₈alkoxy substituted with C₆₋₁₄aryl; OR^{a} (wherein R^{a} is C₁₈alkyl); or NR^{a}R^{b} (wherein R^{a} and R^{b} are independently hydrogen or C(O)R^{c} wherein R^{c} is C₁₋₈alkyl);
R² is C₁₋₈alkyl;
R³ is C₁₋₈alkyl optionally substituted with OR^{a} (wherein R^{a} is hydrogen or C₁₋₈alkyl);
or a pharmaceutically acceptable salt thereof.

9. A compound selected from the group consisting of:
7-(2-Benzyloxy-4-fluoro-benzyl)-4-hydroxy-1-methyl-2-oxo-1,2-dihydro-[1,5]naphthyridine-3-carboxylic acid hydroxymethyl-amide;
7-(2-Benzyloxy-4-fluoro-benzyl)-4-hydroxy-1-methyl-2-oxo-1,2-dihydro-[1,5]naphthyridine-3-carboxylic acid methoxymethyl-amide;
7-(2-Carbamoylmethoxy-4-fluoro-benzyl)-4-hydroxy-1-methyl-2-oxo-1,2-dihydro-[1,5]naphthyridine-3-carboxylic acid (2-hydroxy-ethyl)-amide;
7-(2-Carbamoylmethoxy-4-fluoro-benzyl)-4-hydroxy-1-methyl-2-oxo-1,2-dihydro-[1,5]naphthyridine-3-carboxylic acid (2-methoxy-ethyl)-amide;
7-(4-Fluoro-2-methylcarbamoylmethoy-benzyl)-4-hydroxy-1-methyl-2-oxo-1,2-dihydro-[1,5]naphthyridine-3-carboxylic acid (2-hydroxy-ethyl)-amide;
7-(4-Fluoro-2-methylcarbamoylmethoxy-benzyl)-4-hydroxy-1-methyl-2-oxo-1,2-dihydro-[1,5]naphthyridine-3-carboxylic acid (2-methoxy-ethyl)-amide;
7-(2-Dimethylcarbamoylmethoxy-4-fluoro-benzyl)-4-hydroxy-1-methyl-2-oxo-1,2-dihydro-[1,5]naphthyridine-3-carboxylic acid (2-hydroxy-ethyl)-amide;
7-(2-Dimethylcarbamoylmethoxy-4-fluoro-benzyl)-4-hydroxy-1-methyl-2-oxo-1,2-dihydro-[1,5]naphthyridine-3-carboxylic acid (2-methoxy-ethyl)-amide;
7-[4-Fluoro-2-(2-morpholin-4-yl-ethoxy)-benzyl]-4-hydroxy-1-methyl-2-oxo-1,2-dihydro-[1,5]naphthyridine-3-carboxylic acid (2-hydroxy-ethyl)-amide;
7-[2-(2-Dimethylamino-ethoxy)-4-fluoro-benzyl]-4-hydroxy-1-methyl-2-oxo-1,2-dihydro-[1,5]naphthyridine-3-carboxylic acid (2-hydroxy-ethyl)-amide;
7-[4-Fluoro-2-(2-morpholin-4-yl-ethoxy)-benzyl]-4-hydroxy-1-methyl-2-oxo-1,2-dihydro-[1,5]naphthyridine-3-carboxylic acid (2-methoxy-ethyl)-amide;
7-[2-(2-Dimethylamino-ethoxy)-4-fluoro-benzyl]-4-hydroxy-1-methyl-2-oxo-1,2-dihydro-[1,5]naphthyridine-3-carboxylic acid (2-methoxy-ethyl)-amide;
7-[4-Fluoro-2-(3-methoxy-prop-1-ynyl)-benzyl]-4-hydroxy-1-methyl-2-oxo-1,2-dihydro-[1,5]naphthyridine-3-carboxylic acid (2-hydroxy-ethyl)-amide;
7-[2-(3-Acetylamino-prop-1-ynyl)-4-fluoro-benzyl]-4-hydroxy-1-methyl-2-oxo-1,2-dihydro-[1,5]naphthyridine-3-carboxylic acid (2-hydroxy-ethyl)-amide;
7-[4-Fluoro-2-(3-methoxy-prop-1-ynyl)-benzyl]-4-hydroxy-1-methyl-2-oxo-1,2-dihydro-[1,5]naphthyridine-3-carboxylic acid (2-methoxy-ethyl)-amide;
7-[2-(3-Acetylamino-prop-1-ynyl)-4-fluoro-benzyl]-4-hydroxy-1-methyl-2-oxo-1,2-dihydro-[1,5]naphthyridine-3-carboxylic acid (2-methoxy-ethyl)-amide;
7-(2-Acetyl-4-fluoro-benzyl)-4-hydroxy-1-methyl-2-oxo-1,2-dihydro-[1,5]naphthyridine-3-carboxylic acid(2-hydroxy-ethyl)-amide;
7-(2-Acetyl-4-fluoro-benzyl)-4-hydroxy-1-methyl-2-oxo-1,2-dihydro-[1,5]naphthyridine-3-carboxylic acid(2-methoxy-ethy)-amide;
7-(4-Fluoro-2-hydroxymethyl-benzyl)-4-hydroxy-1-methyl-2-oxo-12-dihydro-[1,5]naphthyridine-3-carboxylic acid (2-hydroxy-ethyl)-amide;
7-(4-Fluoro-2-hydroxymethyl-benzyl)-4-hydroxy-1-methyl-2-oxo-1,2-dihydro-[1,5]naphthyridine-3-carboxylic acid (2-methoxy-ethyl)-amide;
Carbamic acid 5-fluoro-2-[8-hydroxy-7-(2-hydroxy-ethylcarbamoyl)-5-methyl-6-oxo-5,6-dihydro-[1,5]naphthyridin-3-ylmethyl]-benzyl ester;
Carbamic acid 5-fluoro-2-[8-hydroxy-7-(2-methoxy-ethylcarbamoyl)-5-methyl-6-oxo-5,6-dihydro-[1,5]naphthyridin-3-ylmethyl]-benzyl ester;
Ethyl-carbamic acid 5-fluoro-2-[8-hydroxy-7-(2-hydroxy-ethylcarbamoyl)-5-methyl-6-oxo-5,6-dihydro-[1,5]naphthyridin-3-ylmethyl]-benzyl ester;
Ethyl-carbamic acid 5-fluoro-2-[8-hydroxy-7-(2-methoxy-ethylcarbamoyl)-5-methyl-6-oxo-5,6-dihydro-[1,5]naphthyridin-3-ylmethyl]-benzyl ester;
7-(4-Fluoro-2-methoxymethyl-benzyl)-4-methoxy-1-methyl-2-oxo-1,2-dihydro-[1,5]naphthyridine-3-carboxylic acid (2-hydroxy-ethyl)-amide;
7-(2-Carbamoyl-4-fluoro-benzyl)-4-hydroxy-1-methyl-2-oxo-1,2-dihydro-[1,5]naphthyridine-3-carboxylic acid(2-methoxy-ethyl)-amide;
7-(4-Fluoro-2-piperidin-1-ylmethyl-benzyl)-4-hydroxy-1-methyl-2-oxo-1,2-dihydro-[1,5]naphthyridine-3-carboxylic acid hydroxymethyl-amide;
7-(4-Fluoro-2-piperidin-1-ylmethyl-benzyl)-4-hydroxy-1-methyl-2-oxo-1,2-dihydro-[1,5]naphthyridine-3-carboxylic acid methoxymethyl-amide;
7-[4-Fluoro-2-(2-oxo-pyrrolidin-1-ylmethyl)-benzyl]-4-hydroxy-1-methyl-2-oxo-1,2-dihydro-[1,5]naphthyridine-3-carboxylic acid hydroxymethyl-amide;
7-[4-Fluoro-2-(2-oxo-pyrrolidin-1-ylmethyl)-benzyl]-4-hydroxy-1-methyl-2-oxo-1,2-dihydro-[1,5]naphthyridine-3-carboxylic acid methoxymethyl-amide;
7-(4-Fluoro-2-imidazol-1-ylmethyl-benzyl)-4-hydroxy-1-methyl-2-oxo-1,2-dihydro-[1,5]naphthyridine-3-carboxylic acid hydroxymethyl-amide;
7-(4-Fluoro-2-imidazol-1-ylmethyl-benzyl)-4-hydroxy-1-methyl-2-oxo-1,2-dihydro-[1,5]naphthyridine-3-carboxylic acid methoxymethyl-amide;
7-[2-(1,1-Dioxo-1lambda*6*-[1,2]thiazinan-2-ylmethyl)-4-fluoro-benzyl]-4-hydroxy-1-methyl-2-oxo-1,2-dihydro-[1,5]naphthyridine-3-carboxylic acid (2-methoxy-ethyl)-amide;
7-[2-(Acetylamino-methyl)-4-fluoro-benzyl]-4-hydroxy-1-methyl-2-oxo-1,2-dihydro-[1,5]naphthyridine-3-carboxylic acid (2-hydroxy-ethyl)-amide;
7-[4-Fluoro-2-(2-methylcarbamoyl-ethyl)-benzyl]-4-hydroxy-1-methyl-2-oxo-1,2-dihydro-[1,5]naphthyridine-3-carboxylic acid (2-hydroxy-ethyl)-amide;
7-[4-Fluoro-2-(2-methylcarbamoyl-ethyl)-benzyl]-4-hydroxy-1-methyl-2-oxo-1,2-dihydro-[1,5]naphthyridine-3-carboxylic acid (2-methoxy-ethyl)-amide;
7-{2-[2-(Cyanomethyl-methyl-carbamoyl)-ethyl]-4-fluoro-benzyl}-4-hydroxy-1-methyl-2-oxo-1,2-dihydro-[1,5]naphthyridine-3-carboxylic acid (2-hydroxy-ethyl)-amide;
7-{2-[2-(Cyanomethyl-methyl-carbamoyl)-ethyl]-4-fluoro-benzyl}-4-hydroxy-1-methyl-2-oxo-1,2-dihydro-[1,5]naphthyridine-3-carboxylic acid(2-methoxy-ethyl)-amide;
7-[2-(2-Dimethylcarbamoyl-ethyl)-4-fluoro-benzyl]-4-hydroxy-1-methyl-2-oxo-1,2-dihydro-[1,5]naphthyridine-3-carboxylic acid (2-hydroxy-ethyl)-amide;
7-[2-(2-Dimethyicarbamoyl-ethyl)-4-fluoro-benzyl]-4-hydroxy-1-methyl-2-oxo-1,2-dihydro-[1,5]naphthyridine-3-carboxylic acid (2-methoxy-ethyl)-amide;
7-{4-Fluoro-2-[2-(2-hydroxy-1,1-dimethyl-ethylcarbamoyl)-ethyl]-benzyl}-4-hydroxy-1-methyl-2-oxo-1,2-dihydro-[1,5]naphthyridine-3-carboxylic acid (2-hydroxy-ethyl)-amide;
7-{4-Fluoro-2-[2-(2-hydroxy-1,1-dimethyl-ethylcarbamoyl)-ethyl]-benzyl}-4-hydroxy-1-methyl-2-oxo-1,2-dihydro-[1,5]naphthyridine-3-carboxylic acid (2-methoxy-ethyl)-amide;
7-(2-Carbamoyl-4-fluoro-benzyl)-4-hyd roxy-1-methyl-2-oxo-1,2-dihydro-[1,5]naphthyridine-3-carboxylic acid(2-hydroxy-ethyl)-amide;
7-(2-Carbamoyl-4-fluoro-benzyl)-4-hydroxy-1-methyl-2-oxo-1,2-dihydro-[1,5]naphthyridine-3-carboxylic acid(2-methoxy-ethyl)-amide;
7-(4-Fluoro-2-methylcarbamoyl-benzyl)-4-hydroxy-1-methyl-2-oxo-1,2-dihydro-[1,5]naphthyridine-3-carboxylic acid (2-hydroxy-ethyl)-amide;
7-(4-Fluoro-2-methylcarbamoyl-benzyl)-4-hydroxy-1-methyl-2-oxo-1,2-dihydro-[1,5]naphthyridine-3-carboxylic acid (2-methoxy-ethyl)-amide;
7-(2-Dimethylcarbamoyl-4-fluoro-benzyl)-4-hydroxy-1-methyl-2-oxo-1,2-dihydro-[1,5]naphthyridine-3-carboxylic acid (2-hydroxy-ethyl)-amide;
7-(2-Dimethylcarbamoyl-4-fluoro-benzyl)-4-hydroxy-1-methyl-2-oxo-1,2-dihydro-[1,5]naphthyridine-3-carboxylic acid (2-methoxy-ethyl)-amide;
7-[4-Fluoro-2-(4-methyl-piperazine-1-carbonyl)-benzyl]-4-hydroxy-1-methyl-2-oxo-1,2-dihydro-[1,5]naphthyridine-3-carboxylic acid (2-hydroxy-ethyl)-amide;
7-[4-Fluoro-2-(4-methyl-piperazine-1-carbonyl)-benzyl]-4-hydroxy-1-methyl-2-oxo-1,2-dihydro-[1,5]naphthyridine-3-carboxylic acid (2-methoxy-ethyl)-amide;
7-[4-Fluoro-2-(morpholine-4-carbonyl)-benzyl]-4-hydroxy-1-methyl-2-oxo-1,2-dihydro-[1,5]naphthyridine-3-carboxylic acid (2-hydroxy-ethyl)-amide;
7-[4-Fluoro-2-(morpholine-4-carbonyl)-benzyl]-4-hydroxy-1-methyl-2-oxo-1,2-dihydro-[1,5]naphthyridine-3-carboxylic acid (2-methoxy-ethyl)-amide;
7-(2-Ethylcarbamoyl-4-fluoro-benzyl)-4-hydroxy-1-methyl-2-oxo-1,2-dihydro-[1,5]naphthyridine-3-carboxylic acid (2-hydroxy-ethyl)-amide;
7-(2-Ethylcarbamoyl-4-fluoro-benzyl)-4-hydroxy-1-methyl-2-oxo-1,2-dihydro-[1,5]naphthyridine-3-carboxylic acid (2-methoxy-ethyl)-amide;
7-(2-Diethylcarbamoyl-4-fluoro-benzyl)-4-hydroxy-1-methyl-2-oxo-1,2-dihydro-[1,5]naphthyridine-3-carboxylic acid (2-hydroxy-ethyl)-amide;
7-(2-Diethylcarbamoyl-4-fluoro-benzyl)-4-hydroxy-1-methyl-2-oxo-1,2-dihydro-[1,5]naphthyridine-3-carboxylic acid (2-methoxy-ethyl)-amide;
7-(4-Fluoro-2-isopropylcarbamoyl-benzyl)-4-hydroxy-1-methyl-2-oxo-1,2-dihydro-[1,5]naphthyridine-3-carboxylic acid (2-hydroxy-ethyl)-amide;
7-(4-Fluoro-2-isopropylcarbamoyl-benzyl)-4-hydroxy-1-methyl-2-oxo-1,2-dihydro-[1,5]naphthyridine-3-carboxylic acid (2-methoxy-ethyl)-amide;
7-[2-(Ethyl-methyl-carbamoyl)-4-fluoro-benzyl]-4-hydroxy-1-methyl-2-oxo-1,2-dihydro-[1,5]naphthyridine-3-carboxylic acid (2-hydroxy-ethyl)-amide;
7-[2-(Ethyl-methyl-carbamoyl)-4-fluoro-benzyl]-4-hydroxy-1-methyl-2-oxo-1,2-dihydro-[1,5]naphthyridine-3-carboxylic acid (2-methoxy-ethyl)-amide;
7-{2-[Bis-(2-hydroxy-ethyl)-carbamoyl]-4-fluoro-benzyl}-4-hydroxy-1-methyl-2-oxo-1,2-dihydro-[1,5]naphthyridine-3-carboxylic acid (2-hydroxy-ethyl)-amide;
7-{2-[Bis-(2-hydroxy-ethyl)-carbamoyl]-4-fluoro-benzyl}-4-hydroxy-1-methyl-2-oxo-1,2-dihydro-[1,5]naphthyridine-3-carboxylic acid (2-methoxy-ethyl)-amide;
7-[4-Fluoro-2-(methanesulfonylamino-methyl)-benzyl]-4-hydroxy-1-methyl-2-oxo-1,2-dihydro-[1,5]naphthyridine-3-carboxylic acid (2-hydroxyethyl)-amide;
7-[4-Fluoro-2-(methanesulfonylamino-methyl)-benzyl]-4-hydroxy-1-methyl-2-oxo-1,2-dihydro-[1,5]naphthyridine-3-carboxylic acid (2-methoxyethyl)-amide;
7-(4-Fluoro-2-morpholin-4-ylmethyl-benzyl)-4-hydroxy-1-methyl-2-oxo-1,2-dihydro-[1,5]naphthyridine-3-carboxylic acid (2-hydroxy-ethyl)-amide;
7-(2-Dimethylaminomethyl-4-fluoro-benzyl)-4-hydroxy-1-methyl-2-oxo-1,2-dihydro-[1,5]naphthyridine-3-carboxylic acid (2-hydroxy-ethyl)-amide;
7-(4-Fluoro-2-morpholin-4-ylmethyl-benzyl)-4-hydroxy-1-methyl-2-oxo-1,2-dihydro-[1,5]naphthyridine-3-carboxylic acid (2-methoxy-ethyl)-amide;
7-(2-Dimethylaminomethyl-4-fluoro-benzyl)-4-hydroxy-1-methyl-2-oxo-1,2-dihydro-[1,5]naphthyridine-3-carboxylic acid (2-methoxy-ethyl)-amide;
7-{2-[(Acetyl-methyl-amino)-methyl]-4-fluoro-benzyl}-4-hydroxy-1-methyl-2-oxo-1,2-dihydro-[1,5]naphthyridine-3-carboxylic acid (2-hydroxy-ethyl)-amide;
7-{2-[(Acetyl-methyl-amino)-methyl]-4-fluoro-benzyl}-4-hydroxy-1-methyl-2-oxo-1,2-dihydro-[1,5]naphthyridine-3-carboxylic acid (2-methoxy-ethyl)-amide;
7-{4-Fluoro-2-[(methanesulfonyl-methyl-amino)-methyl]-benzyl}-4-hydroxy-1-methyl-2-oxo-1,2-dihydro-[1,5]naphthyridine-3-carboxylic acid (2-hydroxy-ethyl)-amide;
7-{4-Fluoro-2-[(methanesulfonyl-methyl-amino)-methyl]-benzyl}-4-hydroxy-1-methyl-2-oxo-1,2-dihydro-[1,5]naphthyrid ine-3-carboxylic acid (2-methoxy-ethyl)-amide;
7-(2-Dimethylcarbamoylmethyl-4-fluoro-benzyl)-4-hydroxy-1-methyl-2-oxo-1,2-dihydro-[1,5]naphthyridine-3-carboxylic acid (2-hydroxy-ethyl)-amide;
7-(2-Dimethylcarbamoylmethyl-4-fluoro-benzyl)-4-hydroxy-1-methyl-2-oxo-1,2-dihydro-[1,5]naphthyridine-3-carboxylic acid (2-methoxy-ethyl)-amide;
and pharmaceutically acceptable salts thereof.

10. A compound selected from the group consisting of:
7-(2-Benzyloxy-4-fluoro-benzyl)-4-hydroxy-1-methyl-2-oxo-1,2-dihydro-[1,5]naphthyridine-3-carboxylic acid hydroxymethyl-amide;
7-(2-Benzyloxy-4-fluoro-benzyl)-4-hydroxy-1-methyl-2-oxo-1,2-dihydro-[1,5]naphthyridine-3-carboxylic acid methoxymethyl-amide;
7-[4-Fluoro-2-(2-morpholin-4-yl-ethoxy)-benzyl]-4-hydroxy-1-methyl-2-oxo-1,2-dihydro-[1,5]naphthyridine-3-carboxylic acid (2-hydroxy-ethyl)-amide;
7-[4-Fluoro-2-(3-methoxy-prop-1-ynyl)-benzyl]-4-hydroxy-1-methyl-2-oxo-1,2-dihydro-[1,5]naphthyridine-3-carboxylic acid (2-hydroxy-ethyl)-amide;
7-[4-Fluoro-2-(3-methoxy-prop-1-ynyl)-benzyl]-4-hydroxy-1-methyl-2-oxo-1,2-dihydro-[1,5-]naphthyridine-3-carboxylic acid (2-methoxy-ethyl)-amide;
7-(2-Acetyl-4-fluoro-benzyl)-4-hydroxy-1-methyl-2-oxo-1,2-dihydro-[1,5]naphthyridine-3-carboxylic acid(2-hydroxy-ethyl)-amide;
Carbamic acid 5-fluoro-2-[8-hydroxy-7-(2-methoxy-ethylcarbamoyl)-5-methyl-6-oxo-5,6-dihydro-[1,5]naphthyridin-3-ylmethyl]-benzyl ester;
7-(4-Fluoro-2-methoxymethyl-benzyl)-4-hydroxy-1-methyl-2-oxo-1,2-dihydro-[1,5]naphthyridine-3-carboxylic acid (2-hydroxy-ethyl)-amide;
7-[2-(1,1-Dioxo-1lambda*6*-[1,2]thiazinan-2-ylmethyl)-4-fluoro-benzyl]-4-hydroxy-1-methyl-2-oxo-1,2-dihydro-[1,5]naphthyridine-3-carboxylic acid (2-hydroxy-ethyl)-amide;
7-{4-Fluoro-2-[(methanesulfonyl-methyl-amino)-methyl]-benzyl}-4-hydroxy-1-methyl-2-oxo-1,2-dihydro-[1,5]naphthyridine-3-carboxylic acid (2-hydroxy-ethyl)-amide;
and pharmaceutically acceptable salts thereof.

11. A compound selected from the group consisting of 7-(4-Fluoro-2-methoxymethyl-benzyl)-4-methoxy-1-methyl-2-oxo-1,2-dihydro-[1,5]naphthyridine-3-carboxylic acid (2-hydroxy-ethyl)-amide, 7-(4-Fluoro-2-methoxymethyl-benzyl)-4-hydroxy-1-methyl-2-oxo-1,2-dihydro-[1,5]naphthyridine-3-carboxylic acid (2-hydroxy-ethyl)-amide and pharmaceutically acceptable salts thereof.

12. A method of treatment of a viral infection in a human comprising administering to said human an antiviral effective amount of a compound according to claim 1.

13. A method according to claim 12 wherein the viral infection is a HIV infection.

14. (canceled)

15. (canceled)

16. (canceled)

17. A pharmaceutical composition comprising an effective amount of a compound according to claim 1 together with a pharmaceutically acceptable carrier.

18. A pharmaceutical composition according to claim 17 in the form of a tablet or capsule.

19. A pharmaceutical composition according to claim 17 in the form of a liquid or suspension.

20. A method of treatment of a viral infection in a human comprising administering to said human a composition comprising a compound according to claim 1 and another therapeutic agent.

21. The method according to claim 20 wherein the viral infection is an HIV infection.

22. A composition according to claim 17, wherein said composition comprises at least one additional therapeutic agent selected from the group consisting of (1-alpha, 2-beta, 3-alpha)-9-[2,3-bis(hydroxymethyl)cyclobutyl]guanine [(−)BHCG, SQ-34514, lobucavir], 9-[(2R,3R,4S)-3,4-bis(hydroxymethyl)-2-oxetanosyl]adenine (oxetanocin-G), TMC-114, BMS-232632, acyclic nucleosides [e.g. acyclovir, valaciclovir, famciclovir, ganciclovir, penciclovir), acyclic nucleoside phosphonates [e.g. (S)-1-(3-hydroxy-2-phosphonyl-methoxypropyl)cytosine (HPMPC), [[[2-(6-amino-9H-purin-9-yl)ethoxy]methyl]phosphinylidene]bis(oxymethylene)-2,2-dimethylpropanoic acid (bis-POM PMEA, adefovir dipivoxil), [[(1R)-2-(6-amino-9H-purin-9-yl)-1-methylethoxy]methyl]phosphonic acid (tenofovir), (R)-[[2-(6-Amino-9H-purin-9-yl)-1-methylethoxy]methyl]phosphonic acid bis-(isopropoxycarbonyloxymethyl)ester (bis-POC-PMPA)], ribonucleotide reductase inhibitors (e.g. 2-acetylpyridine 5-[(2-chloroanilino)thiocarbonyl) thiocarbonohydrazone and hydroxyurea), nucleoside reverse transcriptase inhibitors (e.g., 3′-azido-3′-deoxythymidine (AZT, zidovudine), 2′,3′-dideoxycytidine (ddC, zalcitabine), 2′,3′-dideoxyadenosine, 2′,3′-dideoxyinosine (ddl, didanosine), 2′,3′-didehydrothymidine (d4T, stavudine), (−)-beta-D-2,6-diaminopurine dioxolane (DAPD), 3′-Azido-2′,3′-dideoxythymidine-5′-H-phosphophonate (phosphonovir), 2′-deoxy-5-iodo-uridine (idoxuridine), as (−)-cis-1-(2-hydroxymethyl)-1,3-oxathiolane 5-yl)-cytosine (lamivudine), or cis-1-(2-(hydroxymethyl)-1,3-oxathiolan-5-yl)-5-fluorocytosine (FTC), 3′-deoxy-3′-fluorothymidine, 5-chloro-2′,3′-dideoxy-3′-fluorouridine, (−)-cis-4-[2-amino-6-(cyclopropylamino)-9H-purin-9-yl]-2-cyclopentene-1-methanol (abacavir), 9-[4-hydroxy-2-(hydroxymethyl)but-1-yl]-guanine (H2G), ABT-606 (2HM-H2G) and ribavirin), protease inhibitors (e.g. indinavir, ritonavir, nelfinavir, amprenavir, saquinavir, (R)-N-tert-butyl-3-[(2S,3S)-2-hydroxy-3-N-[(R)-2-N-(isoquinolin-5-yloxyacetyl)amino-3-methylthiopropanoyl]amino-4-phenylbutanoyl]-5,5-dimethyl-1,3-thiazolidine-4-carboxamide (KNI-272), 4R-(4alpha,5alpha,6beta)]-1,3-bis[(3-aminophenyl)methyl]hexahydro-5,6-dihydroxy-4,7-bis(phenylmethyl)-2H-1,3-diazepin-2-one dimethanesulfonate (mozenavir), 3-[1-[3-[2-(5-trifluoromethylpyridinyl)-sulfonylamino]phenyl]propyl]-4-hydroxy-6alpha-phenethyl-6beta-propyl-5,6-dihydro-2-pyranone (tipranavir), N′-[2(S)-Hydroxy-3(S)-[N-(methoxycarbonyl)-1-tert-leucylamino]-4-phenylbutyl-N^{alpha}-(methoxycarbonyl)-N′-[4-(2-pyridyl)benzyl]-L-tert-leucylhydrazide (BMS-232632), 3-(2(S)-Hydroxy-3(S)-(3-hydroxy-2-methylbenzamido)-4-phenylbutanoyl)-5,5-dimethyl-N-(2-methylbenzyl)thiazolidine-4(R)-carboxamide (AG-1776), N-(2(R)-Hydroxy-1(S)-indanyl)-2(R)-phenyl-methyl-4(S)-hydroxy-5-(1-(1-(4-benzo[b]furanylmethyl)-2(S)-N′-(tert-butylcarboxamido)piperazinyl)pentanamide (MK-944A), and GW 433908), interferons such as α-interferon, renal excretion inhibitors such as probenecid, nucleoside transport inhibitors such as dipyridamole; pentoxifylline, N-acetylcysteine (NAC), Procysteine, α-trichosanthin, phosphonoformic acid, as well as immunomodulators such as interleukin II or thymosin, granulocyte macrophage colony stimulating factors, erythropoetin, soluble CD₄ and genetically engineered derivatives thereof, non-nucleoside reverse transcriptase inhibitors (NNRTIs) for example, TMC-120, TMC-125, nevirapine (BI-RG-587), alpha-((2-acetyl-5-methylphenyl)amino)-2,6-dichloro-benzeneacetamide (loviride), 1-[3-(isopropylamino)-2-pyridyl]-4-[5-(methanesulfonamido)-1H-indol-2-ylcarbonyl]piperazine monomethanesulfonate (delavirdine), (10R, 11S, 12S)-12-Hydroxy-6,6,10,11-tetramethyl-4-propyl-11,12-dihydro-2H, 6H, 10H-benzo(1,2-b:3,4-b′:5,6-b″)tripyran-2-one ((+) calanolide A), (4S)-6-Chloro-4-[1E)-cyclopropylethenyl)-3,4- dihydro-4-(trifluoromethyl)-2(1H)-quinazolinone (DPC-083), 1-(ethoxymethyl)-5-(1-methylethyl)-6-(phenylmethyl)-2,4(1H,3H)-pyrimidinedione (MKC-442), 5-(3,5-dichlorophenyl)thio-4-isopropyl-1-(4-pyridyl)methyl-1H-imidazol-2-ylmethyl carbamate (capravirine), glycoprotein 120 antagonists [e.g. PRO-2000, PRO-542 and 1,4-bis[3-[(2,4-dichlorophenyl)carbonylamino]-2-oxo-5,8-disodiumsulfanyl]naphthalyl-2,5-dimethoxyphenyl-1,4-dihydrazone (FP-21399)], cytokine antagonists [e.g. reticulose (Product-R), 1,1′-azobis-formamide (ADA), and 1,11-(1,4-phenylenebis(methylene))bis-1,4,8,11-tetraazacyclotetradecane octahydrochloride (AMD-3100)], and fusion inhibitors for example T-20 and T-124.

23. A method according to claim 20, wherein said therapeutic agent is selected from the group consisting of (1-alpha, 2-beta, 3-alpha)-9-[2,3-bis(hydroxymethyl)cyclobutyl]guanine [(−)BHCG, SQ-34514, lobucavir], 9-[(2R,3R,4S)-3,4-bis(hydroxymethyl)-2-oxetanosyl]adenine (oxetanocin-G), acyclic nucleosides [e.g. acyclovir, valaciclovir, famciclovir, ganciclovir, penciclovir), acyclic nucleoside phosphonates [e.g. (S)-1-(3-hydroxy-2-phosphonyl-methoxypropyl)cytosine (HPMPC), [[[2-(6-amino-9H-purin-9-yl)ethoxy]methyl]phosphinylidene]bis(oxymethylene)-2,2-dimethylpropanoic acid (bis-POM PMEA, adefovir dipivoxil), [[(1R)-2-(6-amino-9H-purin-9-yl)-1-methylethoxy]methyl]phosphonic acid (tenofovir), (R)-[[2-(6-Amino-9H-purin-9-yl)-1-methylethoxy]methyl]phosphonic acid bis-(isopropoxycarbonyloxymethyl)ester (bis-POC-PMPA)], ribonucleotide red uctase inhibitors (e.g. 2-acetylpyridine 5-[(2-chloroanilino)thiocarbonyl) thiocarbonohydrazone and hydroxyurea), nucleoside reverse transcriptase inhibitors (e.g., 3′-azido-3′-deoxythymidine (AZT, zidovudine), 2′,3′-dideoxycytidine (ddC, zalcitabine), 2′,3′-dideoxyadenosine, 2′,3′-dideoxyinosine (ddl, didanosine), 2′,3′-didehydrothymidine (d4T, stavudine), (−)-beta-D-2,6-diaminopurine dioxolane (DAPD), 3′-Azido-2′,3′-dideoxythymidine-5′-H-phosphophonate (phosphonovir), 2′-deoxy-5-iodo-uridine (idoxuridine), as (−)-cis-1-(2-hydroxymethyl)-1,3-oxathiolane 5-yl)-cytosine (lamivudine), or cis-1-(2-(hydroxymethyl)-1,3-oxathiolan-5-yl)-5-fluorocytosine (FTC), 3′-deoxy-3′-fluorothymidine, 5-chloro-2′,3′-dideoxy-3′-fluorouridine, (−)-cis-4-[2-amino-6-(cyclopropylamino)-9H-purin-9-yl]-2-cyclopentene-1-methanol (abacavir), 9-[4-hydroxy-2-(hydroxymethyl)but-1-yl]-guanine (H2G), ABT-606 (2HM-H2G) and ribavirin), protease inhibitors (e.g. indinavir, ritonavir, nelfinavir, amprenavir, saquinavir, (R)-N-tert-butyl-3-[(2S,3S)-2-hydroxy-3-N-[(R)-2-N-(isoquinolin-5-yloxyacetyl)amino-3-methylthiopropanoyl]amino-4-phenylbutanoyl]-5,5-dimethyl-1,3-thiazolidine-4-carboxamide (KNI-272), 4R-(4alpha,5alpha,6beta)]-1,3-bis[(3-aminophenyl)methyl]hexahydro-5,6-dihydroxy-4,7-bis(phenylmethyl)-2H-1,3-diazepin-2-one dimethanesulfonate (mozenavir), 3-[1-[3-[2-(5-trifluoromethylpyridinyl)-sulfonylamino]phenyl]propyl]-4-hydroxy-6alpha-phenethyl-6beta-propyl-5,6-dihydro-2-pyranone (tipranavir), N′-[2(S)-Hydroxy-3(S)-[N-(methoxycarbonyl)-1-tert-leucylamino]-4-phenylbutyl-N^{alpha}-(methoxycarbonyl)-N′-[4-(2-pyridyl)benzyl]-L-tert-leucylhydrazide (BMS-232632), 3-(2(S)-Hydroxy-3(S)-(3-hydroxy-2-methylbenzamido)-4-phenylbutanoyl)-5,5-dimethyl-N-(2-methylbenzyl)thiazolidine-4(R)-carboxamide (AG-1776), N-(2(R)-Hydroxy-1(S)-indanyl)-2(R)-phenyl-methyl-4(S)-hydroxy-5-(1-(1-(4-benzo[b]furanylmethyl)-2(S)-N′-(tert-butylcarboxamido)piperazinyl)pentanamide (MK-944A), and GW 433908), interferons such as α-interferon, renal excretion inhibitors such as probenecid, nucleoside transport inhibitors such as dipyridamole; pentoxifylline, N-acetylcysteine (NAC), Procysteine, α-trichosanthin, phosphonoformic acid, as well as immunomodulators such as interleukin II or thymosin, granulocyte macrophage colony stimulating factors, erythropoetin, soluble CD₄ and genetically engineered derivatives thereof, non-nucleoside reverse transcriptase inhibitors (NNRTls) [e.g. nevirapine (BI-RG-587), alpha-((2-acetyl-5-methylphenyl)amino)-2,6-dichloro-benzeneacetamide (loviride), 1-[3-(isopropylamino)-2-pyridyl]-4-[5-(methanesulfonamido)-1H-indol-2-ylcarbonyl]piperazine monomethanesulfonate (delavirdine), (10R, 11S, 12S)-12-Hydroxy-6,6,10,11-tetramethyl-4-propyl-11,12-dihydro-2H, 6H, 10H-benzo(1,2-b:3,4-b′:5,6-b″)tripyran-2-one ((+) calanolide A), (4S)-6-Chloro-4-[1E)-cyclopropylethenyl)-3,4- dihydro-4-(trifluoromethyl)-2(1H)-quinazolinone (DPC-083), 1-(ethoxymethyl)-5-(1-methylethyl)-6-(phenylmethyl)-2,4(1H,3H)-pyrimidinedione (MKC-442), 5-(3,5-dichlorophenyl)thio-4-isopropyl-1-(4-pyridyl)methyl-1H-imidazol-2-ylmethyl carbamate (capravirine)], glycoprotein 120 antagonists [e.g. PRO-2000, PRO-542 and 1,4-bis[3-[(2,4-dichlorophenyl)carbonylamino]-2-oxo-5,8-disodiumsulfanyl]naphthalyl-2,5-dimethoxyphenyl-1,4-dihydrazone (FP-21399)], cytokine antagonists [e.g. reticulose (Product-R), 1,1′-azobis-formamide (ADA), and 1,11-(1,4-phenylenebis(methylene))bis-1,4,8,11-tetraazacyclotetradecane octahydrochloride (AMD-3100)], and fusion inhibitors (e.g. T-20 and T-1249).
